(19) United States
(12) Patent Application Publication
(10) Pub. No.: US 2025/0353842 A1
(43) Pub. Date: Nov. 20, 2025

(54) BICYCLIC AMINE CDK12 INHIBITORS

(71) Applicant: Incyte Corporation, Wilmington, DE (US)

(72) Inventors: Evan Styduhar, Claymont, DE (US); Xin Li, Claymont, DE (US); Robert Swyka, Wilmington, DE (US); Oleg Vechorkin, Wilmington, DE (US); Anlai Wang, Wilmington, DE (US); Michael Witten, Philadelphia, PA (US)

(21) Appl. No.: 18/871,262

(22) PCT Filed: Jun. 22, 2023
(86) PCT No.: PCT/US2023/068895

§ 371 Date: Dec. 3, 2024

Related U.S. Application Data
(60) Provisional application No. 63/354,436, filed on Jun. 22, 2022.

(51) Int. Cl. C07D 471/04; A61K 31/506; A61K 31/5365; A61K 31/5377; C07D 403/14; C07D 519/00

(52) U.S. Cl. CPC C07D 471/04; A61K 31/506; A61K 31/5365; A61K 31/5377; C07D 403/14; C07D 519/00

(57) ABSTRACT

The present application provides bicyclic amines that are inhibitors of cyclin-dependent kinase 12 (CDK12), as well as pharmaceutical compositions thereof, and methods of treating cancer using the same

### CROSS-REFERENCE TO RELATED APPLICATIONS

This application claims the benefit of U.S. Provisional Application Ser. No. 63/354,436, filed Jun. 22, 2022, the disclosure of which is incorporated herein by reference in its entirety.

### SEQUENCE LISTING

This application contains a Sequence Listing that has been submitted electronically as an XML file named 20443-0774WO1_SL_ST26.xml. The XML file, created on Jun. 20, 2023, is 2,777 bytes in size. The material in the XML file is hereby incorporated by reference in its entirety.

### TECHNICAL FIELD

This application is directed to bicyclic amines which inhibit cyclin-dependent kinase 12 (CDK12) and are useful for treating cancer.

### BACKGROUND

CDK12 belongs to a family of serine/threonine kinases collectively known as cyclin-dependent kinases (Seung, H. C., et al., Exp. Mol. Med., 2020, 52(5): 762-771). Collectively, CDK's are unique in that they require the binding of specific cyclin proteins for proper functionality (Malumbres, M., et al., Nat. Rev. Cancer., 2009, 9(3): 153-66). Specifically, CDK12 (as well as CDK13) requires the binding of cyclin K in the cyclin binding domain for activation (Kohoutek, J., et al., Cell Div., 2012, 7(12)). Mechanistically, CDK12 and CDK13 phosphorylate serine 2 (pser2) on the C-terminal tail of RNA polymerase II (RNA Pol II), which is required for transcriptional elongation (Bartkowiak, B., et al., Genes Dev, 2010, 24(20): 2303-2316). Therefore, inhibition of CDK12/13 can impact the expression of multiple genes.

Interestingly, CDK12 appears unique among the CDK's in that its inhibition can lead to a selective loss of expression of multiple genes involved in DNA damage repair (Blazek, D., et al., Genes Dev, 2011, 25(20): 2158-2172). Mechanistically, this is attributed to a role of CDK12 in maintaining proper mRNA splicing. Indeed, inhibition or genetic depletion of CDK12 leads to a decrease in proper exon splicing, which in turn increases intronic polyadenylation (IPA) and a subsequent loss of full length mRNA and translated protein (Dubbury, S. J., et al., Nature, 2018, 564(7734): 141-145). Many DNA repair genes are large genes with multiple IPA sites, which explains the selective loss of expression of these repair genes following CDK12 inhibition. Of note, multiple genes involved in the homologous recombination (HR) DNA repair pathway, such as BRCA1 and BRCA2, are especially sensitive to CDK12 inhibition, and indeed inactivating mutations in CDK12 are known to cause a “BRCAness” phenotype in certain cancers (Ekumi, K. M., et al., Nucleic Acids Res, 2015, 43(5): 2575-2589; Wu, Y. M., et al., Cell, 2018, 173(7): 1770-1782).

It is well known that many cancers exhibit defects in various DNA repair pathways; which can confer a selective advantage due to an increased mutation rate (Knijnenburg, T. A., et al., Cell Rep, 2018, 23(1): 239-254). However, these alterations can render cancer cells more susceptible to DNA-damage inducing chemotherapies, or targeted therapies that inhibit additional DNA repair pathways. A well-known example of this paradigm is the increased dependence on the DNA repair enzyme PARP in cancers with defects in HR signaling (i.e., cancers with a “BRCAness” phenotype) (Farmer, H., et al., Nature, 2005, 434(7035): 917-921).

Indeed, preliminary studies have demonstrated that cancers with defective HR exhibit increased sensitivity to pharmacologic or genetic inhibition of CDK12 (Johnson, S. F., et al., Cell Rep., 2016, 17(9): 2367-2381). This therapeutic effect is a consequence of the loss of expression of CDK12-dependent DNA repair genes; which leads to a lethal increase in DNA damage and loss of cell viability (Blazek, D., et al., Genes Dev., 2011, 25(20): 2158-2172).

Despite the clinical emergence of PARP inhibitors as a therapy for patients with HR deficient cancers, de novo resistance or rapid relapse remain an unmet clinical need (Dias, M. P., et al., Nat. Rev. Clin. Oncol., 2021). In the clinic, resistance to PARP inhibitors is most commonly attributed to a reversion to an HR restored tumor, or reliance on additional compensatory DNA repair pathways (Noordermeer, S. M., et al., Trends Cell Biol., 2019, 29(10): 820-834). Similar to a PARP inhibitor, a CDK12 inhibitor is expected to yield the same synthetic lethal interaction in HR deficient tumors. However, given that CDK12 inhibition prevents the expression of HR genes (e.g., BRCA1, BRCA2) it is likely that a CDK12 inhibitor could avoid or overcome the HR-restoration mediate mechanism of resistance observed for PARP inhibitors. Therefore, a CDK12 inhibitor may help fill this unmet clinical need by preventing or overcoming HR restoration during or after PARP inhibitor therapy.

### SUMMARY

The present invention relates to, inter alia, compounds of Formula (I):

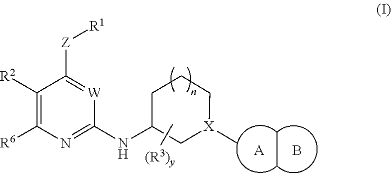

or pharmaceutically acceptable salts thereof, wherein the constituent members are defined herein.

The present invention further provides pharmaceutical compositions comprising a compound described herein, or a pharmaceutically acceptable salt thereof, and a pharmaceutically acceptable carrier.

The present invention further provides methods of inhibiting CDK12, comprising contacting the CDK12 with a compound described herein, or a pharmaceutically acceptable salt thereof.

The present invention further provides methods of inhibiting CDK12 in a patient, comprising administering to the patient a compound described herein, or a pharmaceutically acceptable salt thereof.

The present invention further provides methods of treating a disease or disorder associated with CDK12 in a patient, comprising administering to the patient a compound described herein, or a pharmaceutically acceptable salt thereof.

The present invention further provides compounds described herein, or a pharmaceutically acceptable salt thereof, for use in any of the methods described herein.

The present invention further provides uses of a compound described herein, or a pharmaceutically acceptable salt thereof, for the preparation of a medicament for use in any of the methods described herein.

### DETAILED DESCRIPTION

The present application provides, inter alia, a compound of Formula (I):

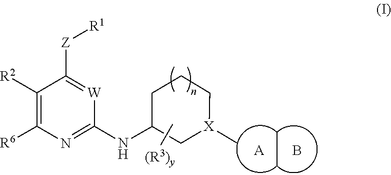

or a pharmaceutically acceptable salt thereof, wherein:
n is 0 or 1;
y is 0, 1, 2, 3, 4, 5, 6, 7, 8, or 9;
W is CH or N;
X is CR³ or N;
Z is NR^{N}, O, S, or absent;
Ring A and Ring B together form a fused bicycle;
Ring A is a 5-membered heteroaryl, which is optionally substituted with 1 or 2 independently selected R⁴ substituents;
or, Ring A is a 5-membered heterocycloalkyl, which is optionally substituted with 1, 2, 3, 4, 5, or 6 independently selected R⁴ substituents;
Ring B is phenyl or a 6-membered heteroaryl, each of which is optionally substituted with 1, 2, 3, or 4 independently selected R⁵ substituents;
R¹ is a 5-membered heteroaryl, which is optionally substituted with 1, 2, 3 or 4 independently selected R^{1A} substituents;
R^{N} is selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{1A} is independently selected from D, halo, CN, NO₂, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h11} and Rⁱ¹¹ are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j11} and R^{k11} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j11} and R^{k11} is independently selected from OH, C₁₋₆ alkoxy, and C₁₋₆ haloalkoxy;
or, any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12} NHOR^{a12}, C(O)R^{b12}, C(O)NR^{c12}R^{d12}, C(O)NR^{c12}(OR^{a12}), C(O)OR^{a12} OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1C} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1C} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or, any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13} NHOR^{a13} C(O)R^{b13}, C(O)NR^{c13}R^{d13}, C(O)NR^{c13}(OR^{a13}), C(O)OR^{a13}, OC(O)R^{b13}, OC(O)NR^{c13}R^{d13}, NR^{c13}R^{d13}, NR^{c13}NR^{c13}R^{d13}, NR^{c13}C(O)R^{b13}, NR^{c13}C(O)OR^{a13}, NR^{c13}C(O)NR^{c13}R^{d13}, C(═NR^{e13})R^{b13}, C(═NR^{e13})NR^{c13}R^{d13}, NR^{c13}C(═NR^{e13})NR^{c13}R^{d13}, NR^{c13}C(═NR^{e13})R^{b13}, NR^{c13}S(O)NR^{c13}R^{d13}, NR^{c13}S(O)R^{b13}, NR^{c13}S(O)₂R^{b13}, NR^{c13}S(O)(═NR^{e13})R^{b13}, NR^{c13}S(O)₂NR^{c13}R^{d13}, S(O)R^{b13}, S(O)NR^{c13}R^{d13}, S(O)₂R^{b13}, S(O)₂NR^{c13}R^{d13}, OS(O)(═NR^{e13})R^{b13}, OS(O)₂R^{b13}, S(O)(═NR^{e13})R^{b13}, SF₅, P(O)R^{f13}R^{g13}, OP(O)(OR^{h13})(ORⁱ¹³), P(O)(OR^{h13})(ORⁱ¹³), and BR^{j13}R^{k13}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a13}, R^{c13} and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c13} and R^{d13} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f13} and R^{g13} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h13} and Rⁱ¹³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j13} and R^{k13} is independently selected from OH, C₁₋₆ alkoxy, and C₁₋₆ haloalkoxy;
or, any R^{j13} and R^{k13} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or, any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{e21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{e21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or, any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R³ is independently selected from H, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a13}, SR^{a13}, NHOR^{a13}, C(O)R^{b3}, C(O)NR^{c3}R^{d3}, C(O)NR^{c3}(OR^{a13}), C(O)OR^{a13}, OC(O)R^{b3}, OC(O)NR^{c3}R^{d3}, NR^{c3}R^{d3}, NR^{c3}NR^{c3}R^{d3}, NR^{c3}C(O)R^{b3}, NR³C(O)OR^{a13}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, S(O)(═NR^{e3})R^{b3}, SF₅, P(O)R^{f3}R^{g3}, OP(O)(OR^{h3})(ORⁱ³), P(O)(OR^{h3})(ORⁱ³), and BR^{j3}R^{k3}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{3A} substituents;
each R^{a13}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3A} substituents;
or, any R^{c3} and R^{d3} attached to the same N atom, together with the N atom to which they are attached, form a 4-10 membered heterocycloalkyl group, which is optionally substituted with 1, 2, 3, or 4 independently selected R^{3A} substituents;
each R^{b3} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j3} and R^{k3} is independently selected from OH, C₁₋₆ alkoxy, and C₁₋₆ haloalkoxy;
or, any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a31}, SR^{a31}, NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31} NR^{c31}C(O)OR^{a31}, NR^{c31}C(O)NR^{c31}R^{d31}, C(═NR^{e31})R^{b31}, C(═NR^{e31})R^{c31}R^{d31}, NR^{c31}C(═NR^{e31})NR^{c31}R^{d31}, NR^{c31}C(═NR^{e31})R^{b31}, NR^{c31}S(O)NR^{c31}R^{d31}, NR^{c31}S(O)R^{b31}, NR^{c31}S(O)₂R^{b31}, NR^{c31}S(O)(═NR^{e31})R^{b31} NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, S(O)(═NR^{e31})R^{b31}, SF₅, P(O)R^{f31}R^{g31}, OP(O)(OR^{h31})(ORⁱ³¹), P(O)(OR^{h31})(ORⁱ³¹), and BR^{j31}R^{k31}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁-6 haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
or, any R^{c31} and R^{d31} attached to the same N atom, together with the N atom to which they are attached, form a 4-7 membered heterocycloalkyl group, which is optionally substituted with 1, 2, 3, or 4 independently selected R^{3B} substituents;
each R^{b31} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3B} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f31} and R^{g31} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h31} and Rⁱ³¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j31} and R^{k31} is independently selected from OH, C₁₋₆ alkoxy, and C₁₋₆ haloalkoxy;
or, any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a32}, SR^{a32}, NHOR^{a32}, C(O)R^{b32}, C(O)NR^{c32}R^{d32}, C(O)NR^{c32}(OR^{a32}), C(O)OR^{a32}, OC(O)R^{b32}, OC(O)NR^{c32}R^{d32}, NR^{c32}R^{d32}, NR^{c32}NR^{c32}R^{d32}, NR^{c32}C(O)R^{b32}, NR^{c32}C(O)OR^{a32}, NR^{c32}C(O)NR^{c32}R^{d32}, C(═NR^{e32})R^{b32}, C(═NR^{e32})NR^{c32}R^{d32}, NR^{c32}C(═NR^{e32})NR^{c32}R^{d32}, NR^{c32}C(═NR^{e32})R^{b32}, NR^{c32}S(O)NR^{c32}R^{d32}, NR^{c32}S(O)R^{b32}, NR^{c32}S(O)₂R^{b32}, NR^{c32}S(O)(═NR^{e32})R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, OS(O)(═NR^{e32})R^{b32}, OS(O)₂R^{b32}, S(O)(═NR^{e32})R^{b32}, SF₅, P(O)R^{f32}R^{g32}, OP(O)(OR^{h32})(ORⁱ³²), P(O)(OR^{h32})(ORⁱ³²), and BR^{j32}R^{k32}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a32}, R^{c32}, and R^{d32} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c32} and R^{d32} attached to the same N atom, together with the N atom to which they are attached, form a 4-7 membered heterocycloalkyl group, which is optionally substituted with 1, 2, 3, or 4 independently selected R^{G} substituents;
each R^{b32} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e32} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f32} and R^{g32} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h32} and Rⁱ³² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j32} and R^{k32} is independently selected from OH, C₁-6 alkoxy, and C₁₋₆ haloalkoxy;
or, any R^{j32} and R^{k32} attached to the same B atom, together with the B atom to which they are attached, form a 5- or 6-membered heterocycloalkyl group optionally substituted with 1, 2, 3, or 4 substituents independently selected from C₁₋₆ alkyl and C₁-6 haloalkyl;
each R⁴ is independently selected from oxo, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4} NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4} NR^{c4}C(═NR^{e4})R^{b4}, NR^{c4}S(O)NR^{c4}R^{d4} NR^{c4}S(O)R^{b4}, NR^{c4}S(O)₂R^{b4} NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R⁴, S(O)₂NR^{c4}R^{d4}, OS(O)(═NR^{e4})R^{b4}, OS(O)₂R^{b4}, S(O)(═NR^{e4})R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c4} and R^{d4} attached to the same N atom, together with the N atom to which they are attached, form a 4-10 membered heterocycloalkyl group, which is optionally substituted with 1, 2, 3, or 4 independently selected R^{4A} substituents;
each R^{b4} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f4} and R^{g4} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h4} and Rⁱ⁴ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a41}, SR^{a41}, NHOR^{a41}, C(O)R^{b41}, C(O)NR^{c41}R^{d41}, C(O)NR^{c41}(OR^{a41}) C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41} NR^{c41}S(O)NR^{c41}R^{d41}, NR^{c41}S(O)R^{b41} NR^{c41}S(O)₂R^{b41}, NR^{c41}S(O)(═NR^{e41})R^{b41} NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, S(O)(═NR^{e41})R^{b41}, SF₄, P(O)R^{f41}R^{g41}, OP(O)(OR^{h41})(ORⁱ⁴¹), P(O)(OR^{h41})(ORⁱ⁴¹), and BR^{j41}R^{k41}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f41} and R^{g41} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h41} and Rⁱ⁴¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j41} and R^{k41} is independently selected from OH, C₁₋₆ alkoxy, and C₁₋₆ haloalkoxy;
or, any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{c42}(OR^{a42}), C(O)OR^{a42} OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{e42}C(═NR^{e42})R^{b42}, NR^{c42}S(O)NR^{c42}R^{d42}, NR^{c42}S(O)R^{b42}, NR^{c42}S(O)₂R^{b42} NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, OS(O)₂R^{b42}, S(O)(═NR^{e42})R^{b42}, SF₅, P(O)R^{f42}R^{g42}, OP(O)(OR^{h42})(ORⁱ⁴²), P(O)(OR^{h42})(ORⁱ⁴²), and BR^{j42}R^{k42}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{e42} and R^{d42} attached to the same N atom, together with the N atom to which they are attached, form a 4-7 membered heterocycloalkyl group, which is optionally substituted with 1, 2, 3, or 4 independently selected R^{G} substituents;
each R^{b42} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f42} and R^{g42} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h42} and Rⁱ⁴² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; each R^{j42} and R^{k42} is independently selected from OH, C₁₋₆ alkoxy, and C₁₋₆ haloalkoxy;
or, any R^{j42} and R^{k42} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}(O)R^{b5}, NR^{c5}(O)OR^{a5}, NR^{c5}(O)NR^{c5}R^{d5}, C(═NR^{e5})R^{b5}, C(═NR^{e5})NR^{c5}R^{d5}, NR^{c5}(═NR^{e5})NR^{c5}R^{d5}, NR^{c5}(═NR^{e5})R^{b5},NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{4A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, W is N.

In some embodiments, W is CH.

In some embodiments, X is CR³.

In some embodiments, each R³ is independently selected from H, D, halo, NO₂, CN, C₁₋₆ alkyl, C₂₋₆ alkenyl, C₂₋₆ alkynyl, and C₁₋₆ haloalkyl.

In some embodiments, each R³ is independently selected from H, D, halo, C₁₋₆ alkyl, and C₁₋₆ haloalkyl.

In some embodiments, each R³ is independently selected from H and C₁₋₆ alkyl.

In some embodiments, each R³ is independently selected from H and C₁₋₃ alkyl.

In some embodiments, X is CH or N.

In some embodiments, X is CH.

In some embodiments, X is N.

In some embodiments, R^{N} is selected from H and C₁₋₆ alkyl.

In some embodiments, Z is NH, O, S, or absent.

In some embodiments, Z is absent.

In some embodiments, Z is NH.

In some embodiments, Z is O.

In some embodiments, Z is S.

In some embodiments, R¹ is a 5-membered heteroaryl, which is substituted with 1, 2, 3, or 4 independently selected R^{1A} substituents.

In some embodiments, R¹ is a 5-membered heteroaryl, which is optionally substituted with 1 or 2 independently selected R^{1A} substituents.

In some embodiments, R¹ is a 5-membered heteroaryl, which is substituted with 1, 2, or 3 independently selected R^{1A} substituents.

In some embodiments, R¹ is a 5-membered heteroaryl, which is substituted with 1 or 2 independently selected R^{1A} substituents.

In some embodiments, R¹ is pyrazolyl, imadazolyl, or triazolyl, each of which is optionally substituted with 1, 2, 3, or 4 independently selected R^{1A} substituents.

In some embodiments, R¹ is pyrazolyl, imadazolyl, or triazolyl, each of which is optionally substituted with 1 or 2 independently selected R^{1A} substituents.

In some embodiments, R¹ is pyrazolyl, imadazolyl, or triazolyl, each of which is substituted with 1, 2, 3, or 4 independently selected R^{1A} substituents.

In some embodiments, R¹ is pyrazolyl, imadazolyl, or triazolyl, each of which is substituted with 1 or 2 independently selected R^{1A} substituents.

In some embodiments, each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{a11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents; or
two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents.

In some embodiments, each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents; and
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, which are each optionally substituted with 1, 2, 3, or 4 independently selected R^{1B} substituents.

In some embodiments, each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}C(O)R^{b11}, NR^{c11}S(O)OR^{a11}, NR^{c11}C(O)NR^{c11}R^{d11}, NR^{c11}S(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents; or
two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents; and
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, which are each optionally substituted with 1, 2, 3, or 4 independently selected R^{1B} substituents.

In some embodiments, each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents; or
two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R¹ substituents.

In some embodiments, each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents.

In some embodiments, each R^{1A} is independently selected from CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R¹ substituents.

In some embodiments, each R^{1A} is independently selected from CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1B} substituents; or
two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents.

In some embodiments, each R^{1A} is independently selected from CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1B} substituents.

In some embodiments, each R^{1A} is independently selected from CN, C₁₋₆ alkyl, C₁₋₆ haloalkyl, and C₃₋₇ cycloalkyl, wherein said C₁₋₆ alkyl, C₁₋₆ haloalkyl, and C₃₋₇ cycloalkyl are each optionally substituted with 1, 2, 3, or 4 independently selected RB substituents.

In some embodiments, each R^{1A} is independently selected from CN, C₁₋₆ alkyl, C₁₋₆ haloalkyl, and C₃₋₇ cycloalkyl, wherein said C₁₋₆ alkyl, C₁₋₆ haloalkyl, and C₃₋₇ cycloalkyl are each optionally substituted with 1, 2, 3, or 4 independently selected RB substituents; or two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected RB substituents.

In some embodiments, each R^{1A} is independently selected from CN, C₁₋₆ alkyl, C₁₋₆ haloalkyl, and C₃₋₇ cycloalkyl, wherein said C₁₋₆ alkyl, C₁₋₆ haloalkyl, and C₃₋₇ cycloalkyl are each optionally substituted with 1, 2, 3, or 4 independently selected RB substituents; or
two R^{1A} substituents, together with the carbon or nitrogen atoms to which they are attached, form a 5- or 6-membered heterocycloalkyl ring, which is optionally substituted with 1, 2, 3, or 4 independently selected R^{1B} substituents.

In some embodiments, two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents.

In some embodiments, two R^{1A} substituents, together with the carbon or nitrogen atoms to which they are attached, form a 5- or 6-membered heterocycloalkyl ring, which is optionally substituted with 1, 2, 3, or 4 independently selected RB substituents.

In some embodiments, each RB is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)NR^{e12}(OR^{a12}), C(O)OR^{a12}, OC(O)R^{b12}, OC(O)NR^{c12}R^{d12}, NR^{c12}R^{d12}, NR^{e12}C(O)R^{b12}, NR^{c12}C(O)OR^{a12}, NR^{c12}C(O)NR^{c12}R^{d12}, NR^{c12}S(O)NR^{c12}R^{d12}, NR^{c12}S(O)R^{b12}, NR^{c12}S(O)₂R^{b12}, NR^{c12}S(O)₂NR^{c12}R^{d12}, S(O)R^{b12}, S(O)NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents.

In some embodiments, each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
or, any R^{c12} and R^{d12} attached to the same N atom, together with the N atom to which they are attached, form a 4-7 membered heterocycloalkyl group; and
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

In some embodiments, each RB is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)NR^{c12}R^{d12}, NR^{c12}S(O)R^{b12}, NR^{c12}S(O)₂R^{b12}, NR^{c12}S(O)₂NR^{c12}R^{d12}, S(O)R^{b12}, S(O)NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
or, any R^{e12} and R^{d12} attached to the same N atom, together with the N atom to which they are attached, form a 4-7 membered heterocycloalkyl group; and each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

In some embodiments, each R^{a12}, R^{b12}, R^{c12}, and R^{d12} is independently selected from H, C₁₋₆ alkyl, C₁₋₆ haloalkyl, C₂₋₆ alkenyl, and C₂₋₆ alkynyl.

In some embodiments, each R^{a12}, R^{c12}, and R^{d12}, is independently selected from H, C₁₋₆ alkyl, C₁₋₆ haloalkyl, C₂₋₆ alkenyl, and C₂₋₆ alkynyl.

In some embodiments, each R^{a12}, R^{b12}, R^{c12}, and R^{d12} is independently selected from H and C₁₋₆ alkyl.

In some embodiments, each R^{a12}, R^{c12}, and R^{d12}, is independently selected from H and C₁₋₆ alkyl.

In some embodiments, each R^{1B} is independently selected from halo, CN, NO₂, C₁₋₆ alkyl, C₂₋₆ alkenyl, C₂₋₆ alkynyl, C₁₋₆ haloalkyl, and OR^{a12} In some embodiments, each R^{1B} is independently selected from halo, CN, NO₂, C₁₋₆ alkyl, C₂₋₆ alkenyl, C₂₋₆ alkynyl, C₁₋₆ haloalkyl, and OR^{a12}; and
each R^{a12}, R^{c12}, and R^{d12}, is independently selected from H, C₁₋₆ alkyl, C₁₋₆ haloalkyl, C₂₋₆ alkenyl, and C₂₋₆ alkynyl.

In some embodiments, each R^{1B} is independently OR^{a12}.

In some embodiments, each R^{1B} is independently OR^{a12}, and each R^{a12} is independently selected from H and C₁₋₆ alkyl.

In some embodiments, each R^{1B} is hydroxy.

In some embodiments, R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR^{c2}S(O)R^{b2} NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents.

In some embodiments, R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
or, any R^{c2} and R^{d2} attached to the same N atom, together with the N atom to which they are attached, form a 4-10 membered heterocycloalkyl group; and
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl.

In some embodiments, R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR²S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
or, any R^{c2} and R^{d2} attached to the same N atom, together with the N atom to which they are attached, form a 4-10 membered heterocycloalkyl group; and
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl.

In some embodiments, each R^{2A} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR²S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
or, any R^{c2} and R^{d2} attached to the same N atom, together with the N atom to which they are attached, form a 4-10 membered heterocycloalkyl group; and
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl.

In some embodiments, R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl.

In some embodiments, R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, and 5-10 membered heteroaryl.

In some embodiments, R² is selected from H, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl.

In some embodiments, R² is selected from H, halo, CN, C₁₋₆ alkyl, and C₁₋₆ haloalkyl.

In some embodiments, R² is selected from halo, CN, and C₁₋₆ haloalkyl.

In some embodiments, R² is selected from C₁, CN, and CF₃.

In some embodiments, y is 0, 1, or 2.

In some embodiments, y is 1.

In some embodiments, y is 0.

In some embodiments, n is 0.

In some embodiments, n is 1.

In some embodiments, Ring A is a 5-membered heteroaryl, which is optionally substituted with 1 or 2 independently selected R⁴ substituents.

In some embodiments, Ring A is a 5-membered heterocycloalkyl, which is optionally substituted with 1, 2, 3, 4, 5, or 6 independently selected R⁴ substituents.

In some embodiments, Ring A is a 5-membered heterocycloalkyl, which is optionally substituted with 1 or 2 independently selected R⁴ substituents.

In some embodiments, Ring A is a 5-membered heteroaryl.

In some embodiments, Ring A is imidazolyl or pyrrolyl, which are each optionally substituted with 1 or 2 independently selected R⁴ substituents.

In some embodiments, Ring A is imidazolyl or pyrrolyl.

In some embodiments, Ring A is imidazolyl.

In some embodiments, Ring A is pyrrolyl.

In some embodiments, Ring B is phenyl or a 6-membered heteroaryl, each of which is optionally substituted with 1 or 2 independently selected R⁵ substituents.

In some embodiments, Ring B is phenyl or pyridyl, each of which is optionally substituted with 1, 2, 3, or 4 independently selected R⁵ substituents.

In some embodiments, Ring B is phenyl or pyridyl, each of which is optionally substituted with 1 or 2 independently selected R⁵ substituents.

In some embodiments, the moiety

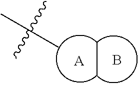

is selected from:

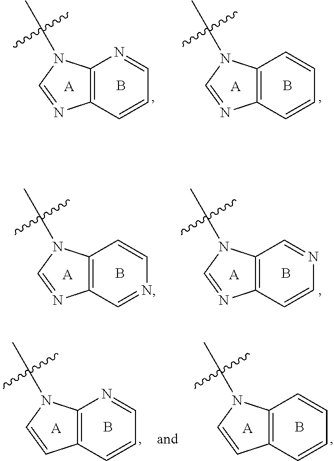

wherein Ring A is optionally substituted with 1 or 2 independently selected R⁴ substituents; and wherein Ring B is optionally substituted with 1 or 2 independently selected R⁵ substituents.

In some embodiments, the moiety

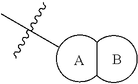

is selected from:

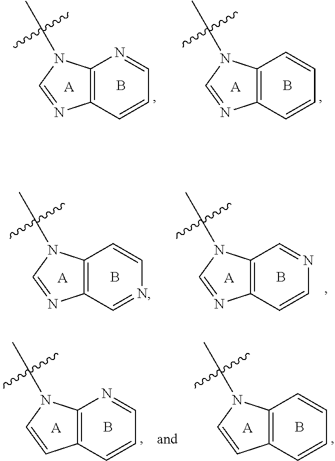

wherein Ring B is optionally substituted with 1 or 2 independently selected R⁵ substituents. In some embodiments of the previous embodiment, W is N. In some embodiments of the previous embodiment, W is CH.

In some embodiments, the moiety

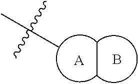

is selected from:

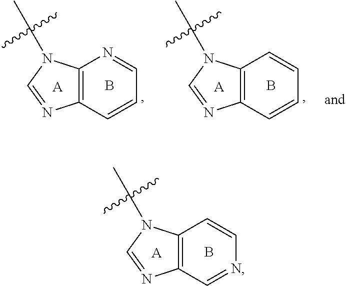

wherein Ring B is optionally substituted with 1 or 2 independently selected R⁵ substituents. In some embodiments of the previous embodiment, W is N. In some embodiments of the previous embodiment, W is CH.

In some embodiments, the moiety

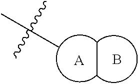

is

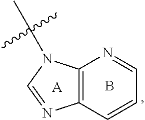

wherein Ring B is optionally substituted with 1 or 2 independently selected R⁵ substituents. In some embodiments of the previous embodiment, W is N. In some embodiments of the previous embodiment, W is CH.

In some embodiments, the moiety

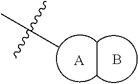

is

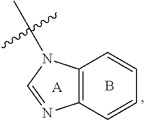

wherein Ring B is optionally substituted with 1 or 2 independently selected R⁵ substituents. In some embodiments of the previous embodiment, W is N. In some embodiments of the previous embodiment, W is CH.

In some embodiments, the moiety

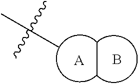

is

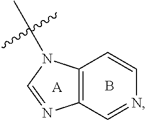

wherein Ring B is optionally substituted with 1 or 2 independently selected R⁵ substituents. In some embodiments of the previous embodiment, W is N. In some embodiments of the previous embodiment, W is CH.

In some embodiments, each R⁴ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}(O)R^{b5}, NR^{c5}(O)OR^{a5}, NR^{c5}(O)NR^{c5}R^{d5}, NR^{c5}S(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents.

In some embodiments, each R⁵ is independently selected from halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, C(O)R^{b5}, and C(O)OR^{a5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents.

In some embodiments, each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, and 5-10 membered heteroaryl are each optionally substituted with 1, 2, 3, or 4 independently selected R^{4A} substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{4A} substituents; and
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl, which are each optionally substituted with 1, 2, 3, or 4 independently selected R^{4A} substituents.

In some embodiments, each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
or, any R^{c5} and R^{d5} attached to the same N atom, together with the N atom to which they are attached, form a 4-7 membered heterocycloalkyl group; and
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

In some embodiments, each R^{a5}, R^{b5}, R^{c5}, and R^{d5} is independently selected from H, C₁₋₆ alkyl, C₁₋₆ haloalkyl, C₂₋₆ alkenyl, and C₂₋₆ alkynyl.

In some embodiments, each R^{a5}, R^{b5}, R^{c5}, and R^{d5} is independently selected from H and C₁₋₆ alkyl.

In some embodiments, each R^{a5} and R^{b5} is independently selected from H, C₁₋₆ alkyl, C₁₋₆ haloalkyl, C₂₋₆ alkenyl, and C₂₋₆ alkynyl.

In some embodiments, each R^{a5} and R^{b5} is independently selected from H and C₁₋₆ alkyl.

In some embodiments, each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}(O)R^{b5}, NR^{c5}(O)OR^{a5}, NR^{c5}(O)NR^{c5}R^{d5}, NR^{c5}S(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, and 5-10 membered heteroaryl are each optionally substituted with 1, 2, 3, or 4 independently selected R^{4A} substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{4A} substituents; and
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl, which are each optionally substituted with 1, 2, 3, or 4 independently selected R^{4A} substituents.

In some embodiments, each R⁵ is independently selected from halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, C(O)R^{b5}, and C(O)OR^{a5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
or, any R^{c5} and R^{d5} attached to the same N atom, together with the N atom to which they are attached, form a 4-7 membered heterocycloalkyl group; and each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

In some embodiments, each R⁵ is independently selected from halo, NO₂, CN, C₁₋₆ alkyl, C₂₋₆ alkenyl, C₂₋₆ alkynyl, C₁₋₆ haloalkyl, OR^{a5}, C(O)R^{b5}, and C(O)OR^{a5}, wherein said C₁₋₆ alkyl, C₂₋₆ alkenyl, C₂₋₆ alkynyl, and C₁₋₆ haloalkyl are each optionally substituted by 1, 2, 3, or 4 independently selected R^{4A} substituents.

In some embodiments, each R⁵ is independently selected from halo, NO₂, CN, C₁₋₆ alkyl, C₂₋₆ alkenyl, C₂₋₆ alkynyl, C₁₋₆ haloalkyl, OR^{a5}, C(O)R^{b5}, and C(O)OR^{a5}, wherein said C₁₋₆ alkyl, C₂₋₆ alkenyl, C₂₋₆ alkynyl, and C₁₋₆ haloalkyl are each optionally substituted by 1, 2, 3, or 4 independently selected R^{4A} substituents; each Ras and R^{b5} is independently selected from H, C₁₋₆ alkyl, C₁₋₆ haloalkyl, C₂₋₆ alkenyl, and C₂₋₆ alkynyl.

In some embodiments, each R^{4A} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R⁵ is independently selected from CN, C₁₋₆ alkyl, C₁₋₆ haloalkyl, OR^{a5}, and C(O)OR^{a5}.

In some embodiments, each R⁵ is independently selected from CN, C₁₋₆ alkyl, C₁₋₆ haloalkyl, OR^{a5}, and C(O)OR^{a5}; and each Ras and R^{b5} is independently selected from H and C₁₋₆ alkyl.

In some embodiments, R⁶ is selected from H, D, OH, NO₂, CN, halo, C₁₋₃ alkyl, C₂₋₃ alkenyl, C₂₋₃ alkynyl, and C₁₋₃ haloalkyl.

In some embodiments, R⁶ is selected from H and C₁₋₃ alkyl.

In some embodiments, R⁶ is H.

In some embodiments:
n is 0 or 1;
y is 0, 1, 2, 3, 4, 5, 6, 7, 8, or 9;
W is N;
X is CH or N;
Z is NH, O, S, or absent;
Ring A and Ring B together form a fused bicycle;
Ring A is a 5-membered heteroaryl, which is optionally substituted with 1 or 2 independently selected R⁴ substituents;
or, Ring A is a 5-membered heterocycloalkyl, which is optionally substituted with 1, 2, 3, 4, 5, or 6 independently selected R⁴ substituents;
Ring B is phenyl or a 6-membered heteroaryl, each of which is optionally substituted with 1, 2, 3, or 4 independently selected R⁵ substituents;
R¹ is a 5-membered heteroaryl, which is optionally substituted with 1, 2, 3 or 4 independently selected R^{1A} substituents;
each R^{1A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}N^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents; or
two R^{1A} substituents, together with the carbon or nitrogen atoms to which they are attached, form a 5- or 6-membered cycloalkyl or a 5- or 6-membered heterocycloalkyl ring;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each Rⁱ¹¹ and R^{k11} is independently selected from OH, C₁₋₆ alkoxy, and C₁₋₆ haloalkoxy;
or, any Rⁱ¹¹ and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12} NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, R^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1C} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1C} substituents; each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or, any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13} NHOR^{a13}, C(O)R^{b13}, C(O)NR^{c13}R^{d13}, C(O)NR^{c13}(OR^{a13}), C(O)OR^{a13}, OC(O)R^{b13}, OC(O)NR^{c13}R^{d13}, NR^{c13}R^{d13}, NR^{c13}R^{d13}, NR^{c13}C(O)R^{b13}, NR^{c13}C(O)OR^{a13}, NR^{c13}C(O)NR^{c13}R^{d13}, C(═NR^{e13})R^{b13}, C(═NR^{e13})NR^{c13}R^{d13}, NR^{c13}C(═NR^{e13})NR^{c13}R^{d13}, NR^{c13}C(═NR^{e13})R^{b13}, NR^{c13}S(O)NR^{c13}R^{d13}, NR^{c13}S(O)R^{b13}, NR^{c13}S(O)₂R^{b13}, NR^{c13}S(O)(═NR^{e13})R^{b13}, NR^{c13}S(O)₂NR^{c13}R^{d13}, S(O)R^{b13}, S(O)NR^{c13}R^{d13}, S(O)₂R^{b13}, S(O)₂NR^{c13}R^{d13}, OS(O)(═NR^{e13})R^{b13}, OS(O)₂R^{b13}, S(O)(═NR^{e13})R^{b13}, SF₅, P(O)R^{f13}R^{g13}, OP(O)(OR^{h13})(ORⁱ¹³), P(O)(OR^{h13})(ORⁱ¹³), and BR^{j13}R^{k13}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a13}, R^{c13} and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c13} and R^{d13} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f13} and R^{g13} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h13} and Rⁱ¹³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j13} and R^{k13} is independently selected from OH, C₁₋₆ alkoxy, and C₁₋₆ haloalkoxy;
or, any R^{j13} and R^{k13} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2} NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR²C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2} NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or, any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e12})NR^{c21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or, any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R³ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a13}, SR^{a13}, NHOR^{a13}, C(O)R^{b3}, C(O)NR^{c3}R^{d3}, C(O)NR^{c3}(OR^{a13}), C(O)OR^{a13}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a13}, NR^{c3}C(O)NR^{c3}R^{d3}, C(═NR^{e3})R^{b3}, C(═NR^{e3})R^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, S(O)(═NR^{e3})R^{b3}, SF₅, P(O)R^{f3}R^{g3}, OP(O)(OR^{h3})(ORⁱ³), P(O)(OR^{h3})(ORⁱ³), and BR^{j3}R^{k3}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{3A} substituents;
each R^{a13}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3A} substituents;
or, any R^{c3} and R^{d3} attached to the same N atom, together with the N atom to which they are attached, form a 4-10 membered heterocycloalkyl group, which is optionally substituted with 1, 2, 3, or 4 independently selected R^{3A} substituents;
each R^{b3} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j3} and R^{k3} is independently selected from OH, C₁₋₆ alkoxy, and C₁₋₆ haloalkoxy;
or, any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a31}, SR^{a31}, NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31} NR^{c31}C(O)OR^{a31}, NR^{c31}C(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c31}C(═NR^{e31})R^{b31}, NR^{c31}S(O)NR^{c31}R^{d31}, NR^{c31}S(O)R^{b31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, S(O)(═NR^{e31})R^{b31}, SF₅, P(O)R^{f31}R^{g31}, OP(O)(OR^{h31})(ORⁱ³¹), P(O)(OR^{h31})(ORⁱ³¹), and BR^{j31}R^{k31}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁-6 haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
or, any R^{c31} and R^{d31} attached to the same N atom, together with the N atom to which they are attached, form a 4-7 membered heterocycloalkyl group, which is optionally substituted with 1, 2, 3, or 4 independently selected R^{3B} substituents;
each R^{b31} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3B} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f31} and R^{g31} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃-10 cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h31} and Rⁱ³¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j31} and R^{k31} is independently selected from OH, C₁₋₆ alkoxy, and C₁₋₆ haloalkoxy;
or, any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a32}, SR^{a32}, NHOR^{a32}, C(O)R^{b32}, C(O)NR^{c32}R^{d32}, C(O)NR^{c32}(OR^{a32}), C(O)OR^{a32}, OC(O)R^{b32}, OC(O)NR^{c32}R^{d32}, NR^{c32}R^{d32} NR^{c32}NR^{c32}R^{d32} NR^{c32}C(O)R^{b32}, NR^{c32}C(O)OR^{a32}, NR^{c32}C(O)NR^{c32}R^{d32}, C(═NR^{e32})R^{b32}, C(═NR^{e32})NR^{c32}R^{d32}, NR^{c32}C(═NR^{e32})NR^{c32}R^{d32}, NR³²C(═NR^{e32})R^{b32}, NR^{c32}S(O)NR^{c32}R^{d32}, NR^{c32}S(O)R^{b32}, NR^{c32}S(O)₂R^{b32}, NR^{c32}S(O)(═NR^{e32})R^{b32}, NR³²S(O)₂NR^{c32}R^{d32}, S(O)R^{b32}, S(O)NR^{c32}R^{d32}, S(O)₂R^{b32}, S(O)₂NR^{c32}R^{d32}, OS(O)(═NR^{e32})R^{b32}, OS(O)₂R^{b32}, S(O)(═NR^{e32})R^{b32}, SF₅, P(O)R^{f32}R^{g32}, OP(O)(OR^{h32})(ORⁱ³²), P(O)(OR^{h32})(ORⁱ³²), and BR^{j32}R³², wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a32}, R^{c32}, and R^{d32} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c32} and R^{d32} attached to the same N atom, together with the N atom to which they are attached, form a 4-7 membered heterocycloalkyl group, which is optionally substituted with 1, 2, 3, or 4 independently selected R^{G} substituents;
each R^{b32} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e32} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f32} and R^{g32} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h32} and Rⁱ³² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j32} and R^{k32} is independently selected from OH, C₁₋₆ alkoxy, and C₁₋₆ haloalkoxy;
or, any R^{j32} and R^{k32} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁴ is independently selected from oxo, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4} NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4} NR^{c4}C(═NR^{e4})R^{b4}, NR^{c4}S(O)NR^{c4}R^{d4} NR^{c4}S(O)R^{b4}, NR^{c4}S(O)₂R^{b4} NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, S(O)(═NR^{e4})R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c4} and R^{d4} attached to the same N atom, together with the N atom to which they are attached, form a 4-10 membered heterocycloalkyl group, which is optionally substituted with 1, 2, 3, or 4 independently selected R^{4A} substituents;
each R^{b4} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f4} and R^{g4} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h4} and Rⁱ⁴ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a41}, SR^{a41}, NHOR^{a41}, C(O)R^{b41}, C(O)NR^{c41}R^{d41}, C(O)NR^{c41}(OR^{a41}) C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41} NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)NR^{c41}R^{d41}, NR^{c41}S(O)R^{b41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, S(O)(═NR^{e41})R^{b41}, SF₄, P(O)R^{f41}R^{g41}, OP(O)(OR^{h41})(ORⁱ⁴¹), P(O)(OR^{h41})(ORⁱ⁴¹), and BR^{j41}R^{k41}, wherein said C₁₋₆ alkyl, C₂₋₆ alkenyl, C₂₋₆ alkynyl, C₁₋₆ haloalkyl, C₃-10 cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f41} and R^{g41} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h41} and Rⁱ⁴¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j41} and R^{k41} is independently selected from OH, C₁₋₆ alkoxy, and C₁₋₆ haloalkoxy;
or, any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{c42}(OR^{a42}), C(O)OR^{a42} OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)NR^{c42}R^{d42}, NR^{c42}S(O)R^{b42}, NR^{c42}S(O)₂R^{b42} NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, OS(O)₂R^{b42}, S(O)(═NR^{e42})R^{b42}, SF₅, P(O)R^{f42}R^{g42}, OP(O)(OR^{h42})(ORⁱ⁴²), P(O)(OR^{h42})(ORⁱ⁴²), and BR^{j42}R^{k42}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c42} and R^{d42} attached to the same N atom, together with the N atom to which they are attached, form a 4-7 membered heterocycloalkyl group, which is optionally substituted with 1, 2, 3, or 4 independently selected R^{G} substituents;
each R^{b42} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f42} and R^{g42} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h42} and Rⁱ⁴² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j42} and R^{k42} is independently selected from OH, C₁₋₆ alkoxy, and C₁₋₆ haloalkoxy;
or, any R^{j42} and R^{k42} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5C}(O)OR^{a5}, NR^{c5C}(O)NRR^{d5}, C(═NR^{e5})R^{b5}, C(═NR^{e5})NR^{c5}R^{d5}, NR^{c5C}(═NR^{e5})NR^{c5}R^{d5}, NR^{c5C}(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(OR), and BR^{j5}R^{k5}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{4A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁶ is selected from H, D, OH, NO₂, CN, halo, C₁₋₃ alkyl, C₂₋₃ alkenyl, C₂₋₃ alkynyl, and C₁₋₃ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0 or 1;
y is 0, 1, or 2;
W is N;
X is CH or N;
Z is NH, O, S, or absent;
Ring A and Ring B together form a fused bicycle;
Ring A is a 5-membered heteroaryl, which is optionally substituted with 1 or 2 independently selected R⁴ substituents;
Ring B is phenyl or pyridyl, each of which is optionally substituted with 1, 2, 3, or 4 independently selected R⁵ substituents;
R¹ is pyrazolyl, imadazolyl, or triazolyl, each of which is optionally substituted with 1, 2, 3, or 4 independently selected R^{1A} substituents;
each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected RB substituents; or
two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected RB substituents; and
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, which are each optionally substituted with 1, 2, 3, or 4 independently selected RB substituents;
each R^{1B} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)N^{c12}(OR^{a12}), C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)NR^{c12}R^{d12}, NR^{c12}S(O)R^{b12}, NR^{c12}S(O)₂R^{b12}, NR^{c12}S(O)₂NR^{c12}R^{d12}, S(O)R^{b12}, S(O)NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected Ric substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
or, any R^{c12} and R^{d12} attached to the same N atom, together with the N atom to which they are attached, form a 4-7 membered heterocycloalkyl group; and
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
each R^{1C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13}, C(O)R^{b13}, C(O)NR^{c13}R^{d13}, C(O)NR^{c13}(OR^{a13}), C(O)OR^{a13}, OC(O)R^{b13}, OC(O)NR^{c13}R^{d13}, NR^{c13}R^{d13}, NR^{c13}C(O)R^{b13}, NR^{c13}C(O)OR^{a13} NR^{c13}C(O)NR^{c13}R^{d13}, NR^{c13}S(O)NR^{c13}R^{d13}, NR^{c13}S(O)R^{b13}, NR^{c13}S(O)₂R^{b13}, NR^{c13}S(O)₂NR^{c13}R^{d13}, S(O)R^{b13}, S(O)NR^{c13}R^{d13}, S(O)₂R^{b13}, and S(O)₂NR^{c13}R^{d13};
each R^{a13}, R^{c13} and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
or, any R^{c13} and R^{d13} attached to the same N atom, together with the N atom to which they are attached, form a 4-7 membered heterocycloalkyl group;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, NR^{c2}C(O)NR^{c2}R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{e2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
or, any R^{c2} and R^{d2} attached to the same N atom, together with the N atom to which they are attached, form a 4-10 membered heterocycloalkyl group; and
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)NR^{c21}(OR^{a21}), C(O)OR^{a21}, OC(O)R^{b21}, OC(O)NR^{e21}R^{d21}, NR^{c21}R^{d21}, NR^{c21}C(O)R^{b21}, NR^{c21}C(O)OR^{a21}, NR^{e21}C(O)NR^{c21}R^{d21}, NR^{c21}S(O)NR^{c21}R^{d21}, NR^{e21}S(O)R^{b21}, NR^{c21}S(O)₂R^{b21}, NR^{c21}S(O)₂NR^{c21}R^{d21}, S(O)R^{b21}, S(O)NR^{e21}R^{d21}, S(O)₂R^{b21}, and S(O)₂NR^{c21}R^{d21};
each R^{a21}, R^{c21} and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
or, any R^{c21} and R^{d21} attached to the same N atom, together with the N atom to which they are attached, form a 4-7 membered heterocycloalkyl group;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R⁴ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}(O)R^{b5}, NR^{c5}(O)OR^{a5}, NR^{c5}(O)NR^{c5}R^{d5}, NR^{c5}S(O)NR^{c5}R^{d5}, NR^{c5}S(O)R^{b5}, NR^{c5}S(O)₂R^{b5}, NR^{c5}S(O)₂NR^{c5}R^{d5} S(O)R^{b5}, S(O)NR^{c5}R^{d5}, S(O)₂R^{b5}, and S(O)₂NR^{c5}R^{d5};
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl;
or, any R^{c5} and R^{d5} attached to the same N atom, together with the N atom to which they are attached, form a 4-10 membered heterocycloalkyl group;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl; and
R⁶ is selected from H and C₁₋₃ alkyl.

In some embodiments, the compound of Formula (I) is a compound of Formula (Ia):

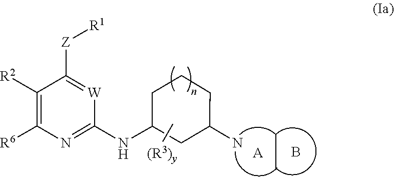

or a pharmaceutically acceptable salt thereof. In some embodiments of Formula (Ia), W is N. In some embodiments of Formula (Ia), W is CH.

In some embodiments of Formula (Ia), the moiety

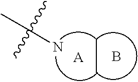

of Formula (Ia) is selected from:

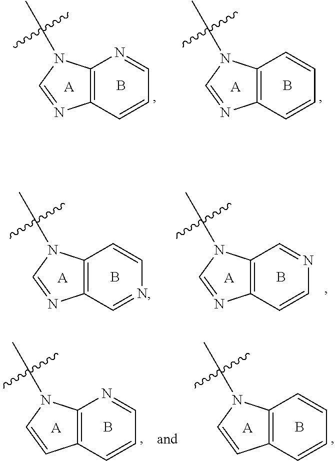

wherein Ring A is optionally substituted with 1 or 2 independently selected R⁴ substituents; and wherein Ring B is optionally substituted with 1 or 2 independently selected R⁵ substituents. In some embodiments of Formula (Ia), W is N. In some embodiments of Formula (Ia), W is CH.

In some embodiments of Formula (Ia), the moiety

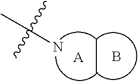

of Formula (Ia) is selected from:

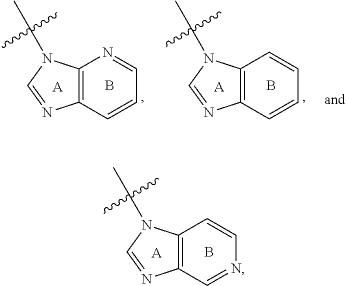

wherein Ring B is optionally substituted with 1 or 2 independently selected R⁵ substituents. In some embodiments of Formula (Ia), W is N. In some embodiments of Formula (Ia), W is CH.

In some embodiments of Formula (Ia), the moiety

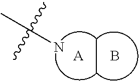

### of Formula (Ia) is

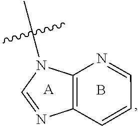

wherein Ring B is optionally substituted with 1 or 2 independently selected R⁵ substituents. In some embodiments of Formula (Ia), W is N. In some embodiments of Formula (Ia), W is CH.

In some embodiments of Formula (Ia), the moiety

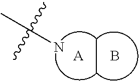

### of Formula (Ia) is

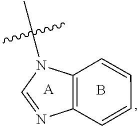

wherein Ring B is optionally substituted with 1 or 2 independently selected R⁵ substituents. In some embodiments of Formula (Ia), W is N. In some embodiments of Formula (Ia), W is CH.

In some embodiments of Formula (Ia), the moiety

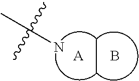

### of Formula (Ia) is

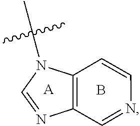

wherein Ring B is optionally substituted with 1 or 2 independently selected R⁵ substituents. In some embodiments of Formula (Ia), W is N. In some embodiments of Formula (Ia), W is CH.

In some embodiments of Formula (Ia), the moiety

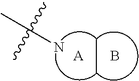

of Formula (Ia) is selected from

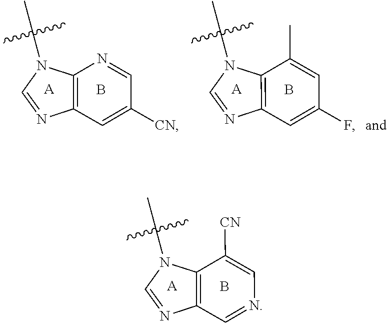

In some embodiments of Formula (Ia), W is N. In some embodiments of Formula (Ia), W is CH.

In some embodiments of Formula (Ia), the moiety

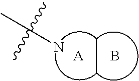

### of Formula (Ia) is

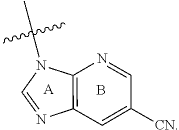

In some embodiments of Formula (Ia), W is N. In some embodiments of Formula (Ia), W is CH.

In some embodiments of Formula (Ia), the moiety

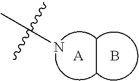

### of Formula (Ia) is

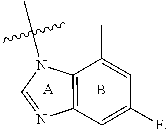

In some embodiments of Formula (Ia), W is N. In some embodiments of Formula (Ia), W is CH.

In some embodiments of Formula (Ia), the moiety

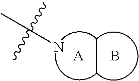

### of Formula (Ia) is

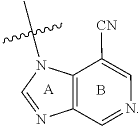

In some embodiments of Formula (Ia), W is N. In some embodiments of Formula (Ia), W is CH.

In some embodiments, the compound of Formula (I) is a compound of Formula (II):

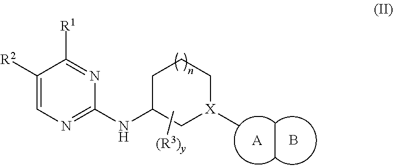

or a pharmaceutically acceptable salt thereof.

In some embodiments, the compound of Formula (I) is a compound of Formula (III):

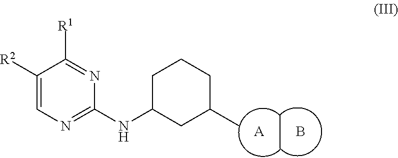

or a pharmaceutically acceptable salt thereof.

In some embodiments, the compound of Formula (I) is a compound of Formula (IV):

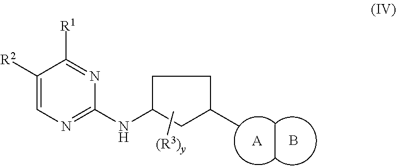

or a pharmaceutically acceptable salt thereof.

In some embodiments, the compound of Formula (I) is a compound of Formula (V):

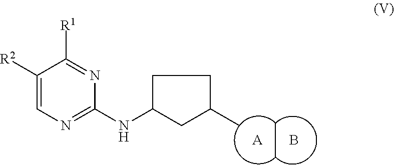

or a pharmaceutically acceptable salt thereof.

In some embodiments, in any of Formulas (II), (III), (IV), or (V), the moiety

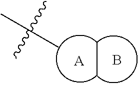

is selected from:

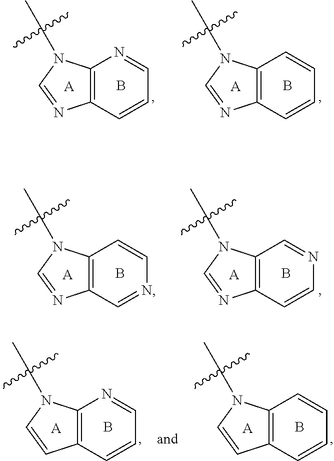

wherein Ring A is optionally substituted with 1 or 2 independently selected R⁴ substituents; and wherein Ring B is optionally substituted with 1 or 2 independently selected R⁵ substituents.

In some embodiments, in any of Formulas (II), (III), (IV), or (V), the moiety

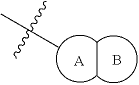

is

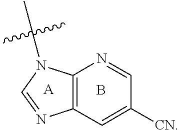

In some embodiments, in any of Formulas (II), (III), (IV), or (V), the moiety

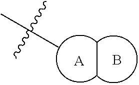

is

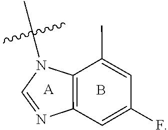

In some embodiments, in any of Formulas (II), (III), (IV), or (V), the moiety

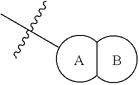

is

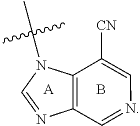

In some embodiments, in any of Formulas (II), (III), (IV), or (V), Z is absent.

In some embodiments, 1, 2, 3, 4, 5, 6, 7, or 8 hydrogen atoms, attached to carbon atoms of “alkyl”, “alkenyl”, “alkynyl”, “aryl”, “phenyl”, “cycloalkyl”, “heterocycloalkyl”, or “heteroaryl” substituents or “—C₁₋₄ alkyl-” and “alkylene” linking groups, as described herein, are optionally replaced by deuterium atoms.

It is further appreciated that certain features of the invention, which are, for clarity, described in the context of separate embodiments, can also be provided in combination in a single embodiment (as if the embodiments were written in multiply dependent form). Conversely, various features of the invention which are, for brevity, described in the context of a single embodiment, can also be provided separately or in any suitable subcombination.

At various places in the present specification, divalent linking substituents are described. Unless otherwise specified, it is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1, 2, 3, 4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency, that the designated atom's normal valency is not exceeded, and that the substitution results in a stable compound.

As used herein, the term “independently selected from” means that each occurrence of a variable or substituent are independently selected at each occurrence from the applicable list.

As used herein, the phrase “each ‘variable’ is independently selected from” means substantially the same as wherein “at each occurrence ‘variable’ is selected from.”

When any variable (e.g., R^{G}) occurs more than one time in any constituent or formula for a compound, its definition at each occurrence is independent of its definition at every other occurrence. Thus, for example, if a group is shown to be substituted with 1, 2, 3, or 4 independently selected R^{G} substituents, then said group may optionally be substituted with up to four R^{G} groups and R^{G} at each occurrence is selected independently from the definition of R^{G}.

In some embodiments, when an optionally multiple substituent is designated in the form:

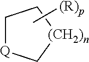

then it is to be understood that substituent R can occur p number of times on the ring, and R can be a different moiety at each occurrence. It is to be understood that each R group may replace any hydrogen atom attached to a ring atom, including one or both of the (CH₂)ₙ hydrogen atoms. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. In some embodiments, the aryl group has 6 to 10 carbon atoms. In some embodiments, the aryl group is phenyl or naphthyl. In some embodiments, the aryl is phenyl.

As used herein, “halo” refers to F, Cl, Br, or I. In some embodiments, halo is F, Cl, or Br. In some embodiments, halo is F or Cl. In some embodiments, halo is F. In some embodiments, halo is Cl.

As used herein, “Cₙ₋ₘ haloalkoxy” refers to a group of formula —O-haloalkyl having n to m carbon atoms. Example haloalkoxy groups include OCF₃ and OCHF₂.

In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “Cₙ₋ₘ fluoroalkyl” refers to an alkyl group having from one fluoro atom to 2s+1 fluoro atoms, where “s” is the number of carbon atoms in the alkyl group, wherein the alkyl group has n to m carbon atoms. In some embodiments, the alkyl group of the fluoroalkyl has 1 to 6, 1 to 4, or 1 to 3 carbon atoms. Example fluoroalkyl groups include CF₃, C₂F₅, CHF₂, CH₂F, and the like.

As used herein, the term “thio” refers to a group of formula —SH.

As used herein, the term “Cₙ₋ₘ alkylamino” refers to a group of formula —NH(alkyl), wherein the alkyl group has n to m carbon atoms. In some embodiments, the alkyl group of the alkylamino has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group of the alkoxycarbonyl has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group of the alkylcarbonyl has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group of the alkylcarbonylamino has 1 to 6, 1 to 4, or 1 to 3 carbon atoms.

As used herein, the term “Cₙ₋ₘ alkoxycarbonylamino” refers to a group of formula —NHC(O)O(Cₙ₋ₘ alkyl), wherein the alkyl group has n to m carbon atoms. In some embodiments, the alkyl group of the alkoxycarbonylamino has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group of the alkylsulfonylamino has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group of the alkylaminosulfonyl has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group of the dialkylaminosulfonyl has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group of the alkylaminosulfonylamino has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group of the dialkylaminosulfonylamino has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group of the alkylaminocarbonylamino has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group of the dialkylaminocarbonylamino has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group of the alkylcarbamyl has 1 to 6, 1 to 4, or 1 to 3 carbon atoms.

As used herein, the term “Cₙ₋ₘ alkylthio” refers to a group of formula —S-alkyl, wherein the alkyl group has n to m carbon atoms. In some embodiments, the alkyl group of the alkylthio has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group of the alkylsulfinyl has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group of the alkylsulfonyl has 1 to 6, 1 to 4, or 1 to 3 carbon atoms.

As used herein, the term “cyano-Cₙ₋ₘ alkyl” refers to a group of formula —(Cₙ₋ₘ alkylene)-CN, wherein the alkylene group has n to m carbon atoms. As used herein, the term “cyano-C₁₋₆ alkyl” refers to a group of formula —(C₁₋₆ alkylene)-CN. As used herein, the term “cyano-C₁₋₃ alkyl” refers to a group of formula —(C₁₋₃ alkylene)-CN.

As used herein, the term “HO—Cₙ₋ₘ alkyl” refers to a group of formula —(Cₙ₋ₘ alkylene)-OH, wherein the alkylene group has n to m carbon atoms. As used herein, the term “HO—C₁₋₃ alkyl” refers to a group of formula —(C₁₋₃ alkylene)-OH.

As used herein, the term “Cₙ₋ₘ alkoxy-Cₒ₋ₚ alkyl” refers to a group of formula —(Cₙ₋ₘ alkylene)-O(Cop alkyl), wherein the alkylene group has n to m carbon atoms and the alkyl group has o to p carbon atoms. As used herein, the term “CI-6 alkoxy-C₁₋₆ alkyl” refers to a group of formula —(C₁₋₆ alkylene)-O(C₁₋₆ alkyl). As used herein, the term “C₁₋₃ alkoxy-C₁₋₃ alkyl” refers to a group of formula —(C₁₋₃ alkylene)-O(C₁₋₃ alkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group of the dialkylamino independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group of the dialkylcarbamyl independently has 1 to 6, 1 to 4, or 1 to 3 carbon atoms.

As used herein, the term “Cₙ₋ₘ alkylcarbonyloxy” is a group of formula —OC(O)-alkyl, wherein the alkyl group has n to m carbon atoms. In some embodiments, the alkyl group of the alkylcarbonyloxy has 1 to 6, 1 to 4, or 1 to 3 carbon atoms.

As used herein, “aminocarbonyloxy” is a group of formula —OC(O)—NH₂.

As used herein, “Cₙ₋ₘ alkylaminocarbonyloxy” is a group of formula —OC(O)—NH-alkyl, wherein the alkyl group has n to m carbon atoms. In some embodiments, the alkyl group of the alkylaminocarbonyloxy has 1 to 6, 1 to 4, or 1 to 3 carbon atoms.

As used herein, “di(Cₙ₋ₘalkyl)aminocarbonyloxy” is a group of formula —OC(O)—N(alkyl)₂, wherein each alkyl group has, independently, n to m carbon atoms.

In some embodiments, each alkyl group of the dialkylaminocarbonyloxy independently has 1 to 6, 1 to 4, or 1 to 3 carbon atoms.

As used herein “Cₙ₋ₘ alkoxycarbonylamino” refers to a group of formula —NHC(O)—O-alkyl, wherein the alkyl group has n to m carbon atoms.

As used herein, the term “carbamyl” to a group of formula —C(O)NH₂.

As used herein, the term “carbonyl”, employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, or S. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridyl, pyrimidinyl, pyrazinyl, pyridazinyl, pyrrolyl, pyrazolyl, azolyl, oxazolyl, isoxazolyl, thiazolyl, isothiazolyl, imidazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, benzoimidazolyl, benzothiazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). As used herein, the term “partially unsaturated ring” refers to a ring having at least one point of saturation and wherein said ring is non-aromatic. Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. A heterocycloalkyl group containing a partially unsaturated ring can also include a fused aromatic ring attached through any ring-forming atom including a ring-forming atom of the fused aromatic ring, wherein said partially unsaturated ring moiety has at least one point of saturation and wherein said partially unsaturated ring is non-aromatic. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Examples of heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, isoindolinonyl, pyrazolidinyl, oxazolidinyl, thiazolidinyl, imidazolidinyl, azepanyl, benzazapene, 1, 2, 3, 4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, azabicyclo[2.2.1]heptan-7-yl, azabicyclo[2.2.1]heptan-2-yl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon ring members and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, the term “alkylene” refers a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “alkenylene” refers a divalent straight chain or branched alkenyl linking group. Examples of “alkenylene groups” include ethen-1,1-diyl, ethen-1,2-diyl, propen-1,3-diyl, 2-buten-1,4-diyl, 3-penten-1,5-diyl, 3-hexen-1,6-diyl, 3-hexen-1,5-diyl, and the like.

As used herein, the term “alkynylene” refers a divalent straight chain or branched alkynyl linking group. Examples of “alkynylene groups” include propyn-1,3-diyl, 2-butyn-1,4-diyl, 3-pentyn-1,5-diyl, 3-hexyn-1,6-diyl, 3-hexyn-1,5-diyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as P-camphorsulfonic acid. Other resolving agents suitable for fractional crystallization methods include stereoisomerically pure forms of a-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g., hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the Schemes below.

The Schemes below provide general guidance in connection with preparing the compounds of the invention. One skilled in the art would understand that the preparations shown in the Schemes can be modified or optimized using general knowledge of organic chemistry to prepare various compounds of the invention. Starting materials, reagents and intermediates whose synthesis is not described herein are either commercially available, known in the literature, or may be prepared by methods known to one skilled in the art.

Compounds of the Formula 1-A can be prepared using a process as illustrated in Scheme 1. In the process depicted in Scheme 1, compounds of formula 1-1 can react with compounds 1-2 via nucleophilic aromatic substitution reactions (e.g., in the presence of a base, such as N,N-diisopropylethylamine) or via standard Buchwald reaction conditions (e.g., in the presence of a palladium catalyst and a suitable base) to give compounds 1-3. Compounds of the formula 1-A can then be generated by cleavage of the nitrogen protecting group (e.g. in the case of Boc, treatment with HCl or TFA). Alternatively, compounds of the formula 1-A can be prepared via reaction of compounds 1-4 and 1-5 via nucleophilic aromatic substitution reactions (e.g., in the presence of a base, such as N,N-diisopropylethylamine) followed by reduction of the nitro group (e.g., in the presence of iron(O) and aqueous ammonium chloride or a suitable palladium catalyst and H₂ gas). Compounds 1-6 can then undergo a condensation reaction with trialkyl orthoformates to forge the AB ring system. Subsequent cleavage of the nitrogen protecting group (e.g. in the case of Boc, treatment with HCl or TFA) then results in compounds of formula 1-A. Alternatively, compounds of the formula 1-A can be prepared via reaction of compounds 1-7 and 1-8 via Mitsunobu reaction [e.g., in the presence of (tributylphosphoranylidene)acetonitrile)] to give compounds 1-9. Compounds of the formula 1-A can then be generated by cleavage of the nitrogen protecting group (e.g. in the case of Boc, treatment with HCl or TFA).

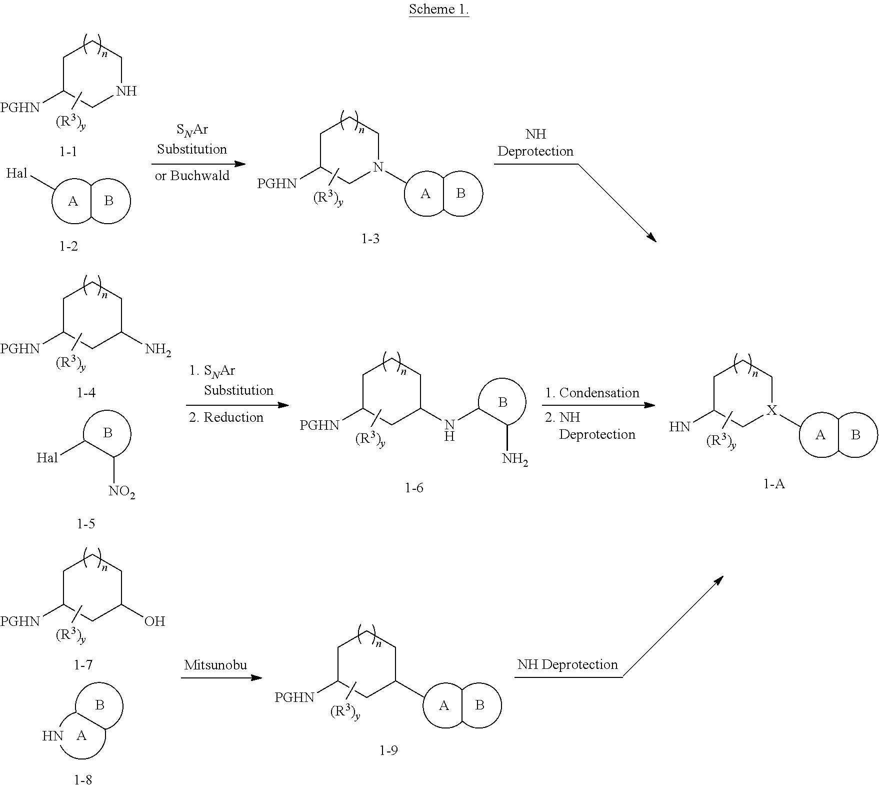

Compounds of the formula 2-A can be prepared using a process as illustrated in Scheme 2. In the process depicted in Scheme 2, compounds of formula 2-1 can react with the compounds of formula 1-A under selective S_{N}Ar conditions (e.g., heating in the presence of zinc(II) chloride) to replace Hal¹ to give compounds of formula 2-2. Compounds of formula 2-2 can undergo coupling to an adduct of formula 2-3, in which M is a boronic acid, a boronic ester or an appropriate reagent [e.g., M is B(OR)₂, Sn(Alkyl)₃, Zn-Hal, etc.], under standard Suzuki cross-coupling conditions (e.g., in the presence of a palladium catalyst and a suitable base) (Tetrahedron 2002, 58, 9633-9695), or standard Stille cross-coupling conditions (e.g., in the presence of a palladium catalyst) (ACS Catalysis 2015, 5, 3040-3053), or standard Negishi cross-coupling conditions (e.g., in the presence of a palladium catalyst) (ACS Catalysis 2016, 6, 1540-1552), to give a derivative of formula 2-A.

Alternatively, if Z is O, NH, or NR^{N}, compounds of the formula 2-A can be prepared by treatment of compounds 2-2 with an adduct of formula 2-4 via nucleophilic aromatic substitution reaction (e.g., in the presence of a base, such as N,N-diisopropylethylamine). Compounds of the formula 2-A can also be prepared by a transmetallation reaction of compounds 2-2 (e.g., in the presence of a suitable palladium catalyst and metallating reagent such as hexamethylditin) to transform Hal² (Hal is a halide such as Cl, Br, or I) into M [B(OR)₂, Sn(Alkyl)₃, Zn-Hal, etc.]. Lastly, compounds 2-5 can undergo a coupling reaction to an adduct of formula 2-6, in which Hal is a halide such as Cl, Br, or I, under standard Suzuki cross-coupling conditions (e.g., in the presence of a palladium catalyst and a suitable base) (Tetrahedron 2002, 58, 9633-9695), or standard Stille cross-coupling conditions (e.g., in the presence of a palladium catalyst) (ACS Catalysis 2015, 5, 3040-3053), or standard Negishi cross-coupling conditions (e.g., in the presence of a palladium catalyst) (ACS Catalysis 2016, 6, 1540-1552), to give a derivative of formula 2-A.

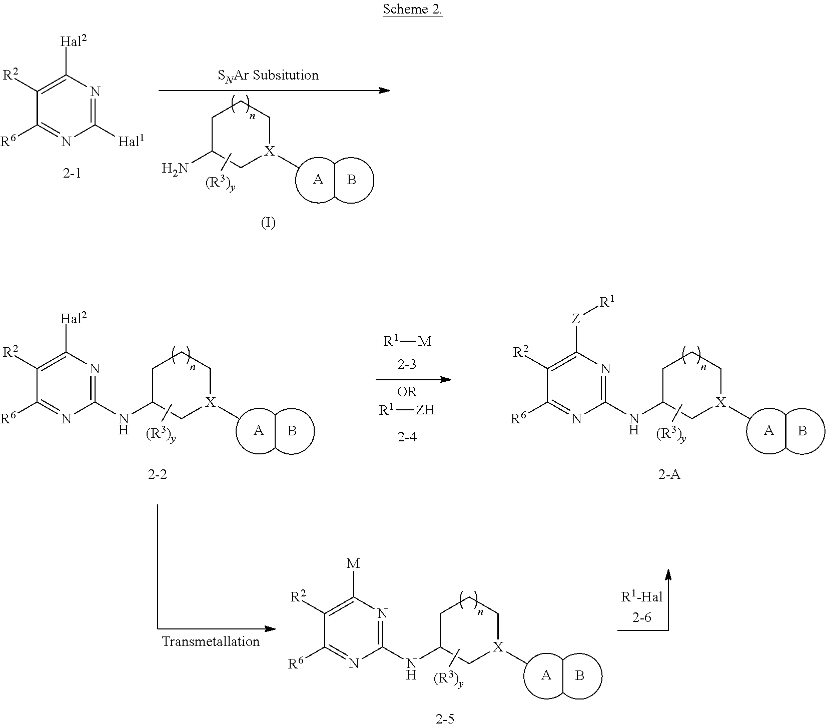

For the synthesis of particular compounds, the general schemes described above can be modified. For example, the products or intermediates can be modified to introduce particular functional groups. Alternatively, the substituents can be modified at any step of the overall synthesis by methods know to one skilled in the art, e.g., as described by Larock, Comprehensive Organic Transformations: A Guide to Functional Group Preparations (Wiley, 1999); and Katritzky et al. (Ed.), Comprehensive Organic Functional Group Transformations (Pergamon Press 1996).

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “r.t.” as used herein, are understood in the art, and refer generally to a temperature, e.g., a reaction temperature, that is about the temperature of the room in which the reaction is carried out, for example, a temperature from about 20° C. to about 30° C.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

### Methods of Use

Compounds of the present disclosure can inhibit CDK12 and therefore are useful for treating diseases wherein the underlying pathology is wholly or partially mediated by CDK12. Such diseases include cancer and other diseases with proliferation disorder. In some embodiments, the present disclosure provides treatment of an individual or a patient in vivo using a compound of Formula (I) or a salt thereof such that growth of cancerous tumors is inhibited. A compound of Formula (I) or of any of the formulas as described herein, or a compound as recited in any of the claims and described herein, or a salt thereof, can be used to inhibit the growth of cancerous tumors with aberrations that activate the CDK12 kinase activity.

Alternatively, a compound of Formula (I) or of any of the formulas as described herein, or a compound as recited in any of the claims and described herein, or a salt thereof, can be used in conjunction with other agents or standard cancer treatments, as described below.

In some embodiments, the present disclosure provides a method for inhibiting growth of tumor cells in vitro. The method includes contacting the tumor cells in vitro with a compound of Formula (I), or any of the formulas as described herein, or a compound as recited in any of the claims and described herein, or a salt thereof.

In some embodiments, provided herein is a method of inhibiting CDK12, comprising contacting the CDK12 with a compound of Formula (I), or any of the formulas as described herein, a compound as recited in any of the claims and described herein, or a salt thereof. In some embodiments, provided herein is a method of inhibiting CDK12 in a patient, comprising administering to the patient a compound of Formula (I), or any of the formulas as described herein, a compound as recited in any of the claims and described herein, or a salt thereof.

In some embodiments, the compounds of the present disclosure are selective inhibitors of CDK12 over one or more other CDKs. For example, some of the compounds described herein, or a pharmaceutically acceptable salts thereof, preferentially inhibit CDK12 over one or more of CDK1, CDK2, CDK4, CDK5, CDK6, CDK7, CDK9, and CDK13 as determined by one or more assays disclosed herein.

In some embodiments, the compounds of the present disclosure are selective inhibitors of CDK12 over one or more of CDK1, CDK2, CDK7, and CDK9.

In some embodiments, the compounds of the present disclosure are selective inhibitors of CDK12 over one or more of CDK1, CDK2, and CDK7.

In some embodiments, provided herein is a method for treating cancer. The method includes administering to a patient (in need thereof), a therapeutically effective amount of a compound of Formula (I), or any of the formulas as described herein, a compound as recited in any of the claims and described herein, or a salt thereof.

In some embodiments, provided herein is a method of treating a disease or disorder associated with CDK12 in a patient, comprising administering to the patient a therapeutically effective amount of a compound of Formula (I), or any of the formulas as described herein, a compound as recited in any of the claims and described herein, or a salt thereof.

In some embodiments, the disease or disorder associated with CDK12 is a cancer.

In some embodiments, the disease or disorder associated with CDK12 is a cancer which has been previously identified as homologous recombination deficiency (HRD) high. In some embodiments, the patient has been identified as a patient having homologous recombination deficiency (HRD). In some embodiments, the patient has been identified as having a positive test result for deleterious or suspected deleterious mutations in BRCA1 or BRCA2 genes. In some embodiments, the patient has been identified as having a positive Genomic Instability Score (see e.g., myChoice® CDx, Myriad Genetics, 2019, https://myriad.com/products-services/precision-medicine/mychoice-cdx/).

In some embodiments, the cancer is ovarian cancer, breast cancer, Ewing's sarcoma, osteosarcoma, liver cancer, hepatocellular carcinoma, or colorectal cancer.

In some embodiments, the cancer is ovarian cancer.

In some embodiments, the cancer is serous ovarian carcinoma.

In some embodiments, the cancer is HRD high grade serous ovarian carcinoma (see Bajrami, I., et al., Cancer Res, 2014. 74(1): 287-297).

In some embodiments, the cancer is breast cancer.

In some embodiments, the cancer is homologous recombination deficient breast cancer (see Johnson, S. F., et al., Cell Rep, 2016. 17(9): 2367-2381).

In some embodiments, the cancer is Ewing's sarcoma (see Iniguez, A. B., et al., Cancer Cell, 2018. 33(2): 202-216).

In some embodiments, the cancer is osteosarcoma (see Bayles, I., et al., JCI, 2019. 129(10): 4377-4392).

In some embodiments, the cancer is liver cancer.

In some embodiments, the cancer is hepatocellular carcinoma (see Wang, C., et al., Gut, 2020. 69(4): 727-736).

In some embodiments, the cancer is colorectal cancer (see Jiang, B., et al., Nat. Chem. Biol., 2021. 17: 675-683; and Dieter, S. M., et al., Cell Rep., 2021, 36, 109394).

In some embodiments, the cancer is uterine carcinosarcoma.

In some embodiments, the cancer is melanoma.

In some embodiments, the cancer is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the cancer is lung adenocarcinoma, breast invasive carcinoma, uterine carcinosarcoma, ovarian serous cystadenocarcinoma, or stomach adenocarcinoma.

In some embodiments, the cancer is an adenocarcinoma, carcinoma, or cystadenocarcinoma.

In some embodiments, the cancer is uterine cancer, ovarian cancer, stomach cancer, esophageal cancer, lung cancer, bladder cancer, pancreatic cancer, or breast cancer.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual”, “patient,” and “subject” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1), inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

The present disclosure further provides a compound described herein (i.e., a compound of Formula (I), or any of the formulas as described herein, a compound as recited in any of the claims and described herein, or a salt thereof), for use in any of the methods described herein.

The present disclosure further provides uses of a compound described herein (i.e., a compound of Formula (I), or any of the formulas as described herein, a compound as recited in any of the claims and described herein, or a salt thereof), for the preparation of a medicament for use in any of the methods described herein.

### Combination Therapies

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of CDK12-associated diseases, disorders or conditions. Other agents such as therapeutic antibodies can be used in combination with the compounds of the present disclosure for treatment of CDK12-associated diseases, disorders or conditions. The one or more additional pharmaceutical agents can be administered to a patient simultaneously or sequentially.

In some embodiments, the CDK12 inhibitor is administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-PR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3K6 inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXARm (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C₂₂₅ (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™) nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK12 inhibitor of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### II. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122.

In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105.

In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8119.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F₉-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFP receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), or more, such as about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action.

For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect.

Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating CDK12 in tissue samples, including human, and for identifying CDK12 activators by inhibition binding of a labeled compound. Substitution of one or more of the atoms of the compounds of the present disclosure can also be useful in generating differentiated ADME (Adsorption, Distribution, Metabolism and Excretion.) Accordingly, the present disclosure includes CDK12 assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups of the disclosed Formulas (e.g., Formula (I)) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃).

In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

Synthetic methods for including isotopes into organic compounds are known in the art (Deuterium Labeling in Organic Chemistry by Alan F. Thomas, New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro CDK12 labeling and competition assays, compounds that incorporate ³H, ¹⁴C, ⁸²Br, ¹²⁵, ¹³¹I, or ³⁵S can be useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present disclosure can further include synthetic methods for incorporating radio-isotopes into compounds of the disclosure. Synthetic methods for incorporating radio-isotopes into organic compounds are well known in the art, and one of ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind and activate CDK12 by monitoring its concentration variation when contacting with CDK12, through tracking of the labeling. For example, a test compound (labeled) can be evaluated for its ability to reduce binding of another compound which is known to inhibit CDK12 (i.e., standard compound). Accordingly, the ability of a test compound to compete with the standard compound for binding to CDK12 directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of CDK12-associated diseases or disorders (such as, e.g., cancer) which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g., “Two-Pump at-Column Dilution Configuration for Preparative LC-MS,” K. Blom, J. Combi. Chem., 4, 295 (2002); “Optimizing Preparative LC-MS Configurations and Methods for Parallel Synthesis Purification,” K. Blom, R. Sparks, J. Doughty, G. Everlof, T. Haque, A. Combs, J. Combi. Chem., 5, 670 (2003); and “Preparative LC-MS Purification: Improved Compound Specific Method Optimization,” K. Blom, B. Glass, R. Sparks, A. Combs, J. Combi. Chem., 6, 874-883 (2004). The separated compounds were typically subjected to analytical liquid chromatography mass spectrometry (LCMS) for purity check under the following conditions: Instrument: Agilent 1100 series, LC/MSD; Column: Waters Sunfire™ C₁₈ 5 μm particle size, 2.1×5.0 mm; Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm particle size, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see “Preparative LCMS Purification: Improved Compound Specific Method Optimization,” K. Blom, B. Glass, R. Sparks, A. Combs, J. Comb. Chem., 6, 874-883 (2004)). Typically, the flow rate used with the 30×100 mm column was 60 mL/minute.

pH=10 purifications: Waters XBridge C₁₈ 5 μm particle size, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (See “Preparative LCMS Purification: Improved Compound Specific Method Optimization,” K. Blom, B. Glass, R. Sparks, A. Combs, J. Comb. Chem., 6, 874-883 (2004)). Typically, the flow rate used with 30×100 mm column was 60 mL/minute.

### Intermediate 1. 4-(1-(2,2-Difluoroethyl)-1H-pyrazol-4-yl)-2-(methylsulfonyl)pyrimidine-5-carbonitrile

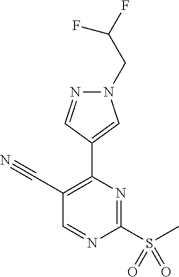 [SMILES: CS(=O)(=O)c1ncc(C#N)c(-c2cnn(CC(F)F)c2)n1]

### Step 1. 4-(1-(2,2-Difluoroethyl)-1H-pyrazol-4-yl)-2-(methylthio)pyrimidine-5-carbonitrile

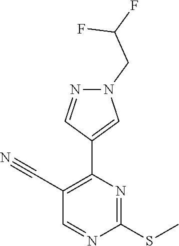 [SMILES: CSc1ncc(C#N)c(-c2cnn(CC(F)F)c2)n1]

A flask containing 4-chloro-2-(methylthio)pyrimidine-5-carbonitrile (2.0 g, 10.8 mmol), 1-(2,2-difluoroethyl)-4-(4,4,5,5-tetramethyl-1,3,2-dioxaborolan-2-yl)-1H-pyrazole (2.8 g, 10.8 mmol), sodium carbonate (3.4 g, 32 mmol) and Pd(dppf)Cl₂ CH₂Cl₂ (394 mg, 0.54 mmol) was evacuated and backfilled with nitrogen three times, followed by the addition of DMF (28 mL) and water (8 mL).

The vessel was heated and stirred at 120° C. for 1 h. The reaction mixture was cooled to rt, poured into ice water, and the resulting solid was collected by filtration. The solid was washed with water, hexanes, and left to dry under vacuum. The obtained crude product was used in the next step without further purification. LCMS calculated for C₁₁H₁₀F₂N₅S (M+H)⁺: m/z=282.1; found: 282.0.

### Step 2. 4-(1-(2,2-Difluoroethyl)-1H-pyrazol-4-yl)-2-(methylsulfonyl)pyrimidine-5-carbonitrile

The crude material from the previous step was dissolved in CH₂Cl₂ (100 mL), followed by the addition of m-CPBA (6.0 g, 27.0 mmol) and left to stir at rt for 1 h. The reaction was diluted with sat. aq. NaHCO₃ (50 mL), then extracted three times with CH₂Cl₂ (100 mL). The combined organic layers were washed with brine (50 mL), dried over MgSO₄, filtered, and concentrated in vacuo. The resulting residue was dryloaded onto 20 g of SiO₂ and purified by Teledyne ISCO CombiFlash (CH₂Cl₂/MeOH, up to 10% MeOH) to give the desired product as an off-white solid (1.67 g, 50% yield over two steps). LCMS calculated for C₁₁H₁₀F₂N₅O₂S (M+H)⁺: m/z=314.1; found: 314.0.

### Example 1. 3-((1S,3R)-3-((4-(1-(2,2-Difluoroethyl)-1H-pyrazol-4-yl)-5-(trifluoromethyl)pyrimidin-2-yl)amino)cyclohexyl)-3H-imidazo[4,5-b]pyridine-6-carbonitrile

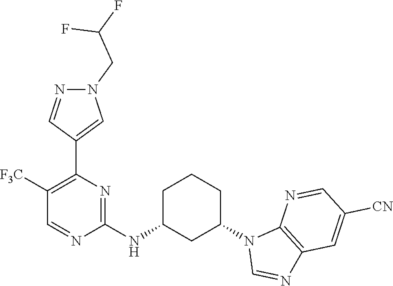 [SMILES: N#Cc1cnc2c(c1)ncn2[C@H]1CCC[C@@H](Nc2ncc(C(F)(F)F)c(-c3cnn(CC(F)F)c3)n2)C1]

### Step 1. tert-Butyl ((1R,3S)-3-((5-cyano-3-nitropyridin-2-yl)amino)cyclohexyl)carbamate

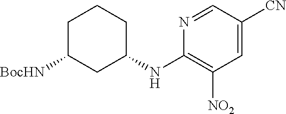 [SMILES: CC(C)(C)OC(=O)N[C@@H]1CCC[C@H](Nc2ncc(C#N)cc2[N+](=O)[O-])C1]

A mixture of tert-butyl ((1R,3S)-3-aminocyclohexyl)carbamate (1.17 g, 5.45 mmol), 6-chloro-5-nitronicotinonitrile (1.0 g, 5.4 mmol), and DIPEA (1.9 mL, 10.9 mmol) in EtOH (10 mL) was heated to 80° C. and stirred for 2 h. Upon completion, the reaction mixture was cooled to rt and concentrated in vacuo. The crude residue was purified by Biotage Isolera (CH₂Cl₂/MeOH, up to 10% MeOH) to give the desired product as yellow solid (1.7 g, 86% yield). LCMS calculated for C₁₃H₁₆N₅O₄ (M+H−C₄H₈)⁺: m/z=306.1; found: 306.1.

### Step 2. tert-Butyl ((1R,3S)-3-((3-amino-5-cyanopyridin-2-yl)amino)cyclohexyl)carbamate

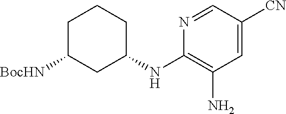 [SMILES: CC(C)(C)OC(=O)N[C@@H]1CCC[C@H](Nc2ncc(C#N)cc2N)C1]

A vial containing tert-butyl ((1R,3S)-3-((5-cyano-3-nitropyridin-2-yl)amino)cyclohexyl)carbamate (1.7 g, 4.7 mmol) and Pd/C (5 wt % loading, Degussa type, 501 mg, 0.47 mmol) as a suspension in EtOH (10 mL) was evacuated and backfilled with an H₂ balloon three times, then stirred under H₂ balloon at rt for 2 h. The reaction mixture was filtered through Celite and the filtrate was concentrated in vacuo to give the desired product as a white solid. LCMS calculated for C₁₇H₂₆N₅O₂ (M+H)⁺: m/z=332.2; found: 332.1.

### Step 3. tert-Butyl ((1R,3S)-3-(6-cyano-3H-imidazo[4,5-b]pyridin-3-yl)cyclohexyl)carbamate

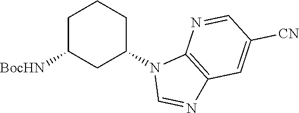 [SMILES: CC(C)(C)OC(=O)N[C@@H]1CCC[C@H](n2cnc3cc(C#N)cnc32)C1]

A vial containing tert-butyl ((1R,3S)-3-((3-amino-5-cyanopyridin-2-yl)amino)cyclohexyl)carbamate (1.4 g, 4.2 mmol), trimethyl orthoformate (2.3 mL, 21.1 mmol), and p-toluenesulfonic acid monohydrate (10 mg, 0.05 mmol) as a solution in toluene (10 mL) was sealed and heated to 90° C. for 2 h. Upon completion, the reaction mixture was concentrated in vacuo. The resulting residue was purified by Biotage Isolera (CH₂Cl₂/MeOH, up to 10% MeOH) to give the product as a colorless solid (1.1 g, 76% yield). LCMS calculated for C₁₈H₂₄N₅O₂ (M+H)⁺: m/z=342.2; found: 342.1.

### Step 4. 3-((1S,3R)-3-Aminocyclohexyl)-3H-imidazo[4,5-b]pyridine-6-carbonitrile

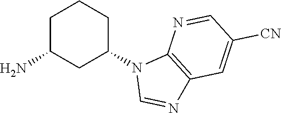 [SMILES: N#Cc1cnc2c(c1)ncn2[C@H]1CCC[C@@H](N)C1]

To a solution of tert-butyl ((1R,3S)-3-(6-cyano-3H-imidazo[4,5-b]pyridin-3-yl)cyclohexyl)carbamate (1.1 g, 3.2 mmol) in MeOH (3 mL) was added 4N HCl/dioxane (5 mL) and left to stir at rt for 1 h. Upon completion, the reaction mixture was concentrated in vacuo. The obtained crude product was used in the next step without further purification. LCMS calculated for C₁₃H₁₆N₅(M+H)⁺: m/z=242.1; found: 242.2.

### Step 5. 3-((1S,3R)-3-((4-(1-(2,2-Difluoroethyl)-1H-pyrazol-4-yl)-5-(trifluoromethyl)pyrimidin-2-yl)amino)cyclohexyl)-3H-imidazo[4,5-b]pyridine-6-carbonitrile

To a solution of 3-((1S,3R)-3-aminocyclohexyl)-3H-imidazo[4,5-b]pyridine-6-carbonitrile (35 mg, 0.15 mmol) in EtOH (1 mL) was added 2-chloro-4-(1-(2,2-difluoroethyl)-1H-pyrazol-4-yl)-5-(trifluoromethyl)pyrimidine (46 mg, 0.15 mmol, Example 15, Step 2) and DIPEA (76 μL, 0.44 mmol). The reaction mixture was stirred at rt for 2 h. Upon completion, the reaction mixture was diluted with CH₃CN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₁F₅N₉ (M+H)⁺: m/z=518.2; found: 518.3.

### Example 2. 3-((1S,3R)-3-((4-(1-(2,2-Difluoroethyl)-1H-pyrazol-4-yl)-5-(trifluoromethyl)pyrimidin-2-yl)amino)cyclopentyl)-3H-imidazo[4,5-b]pyridine-6-carbonitrile

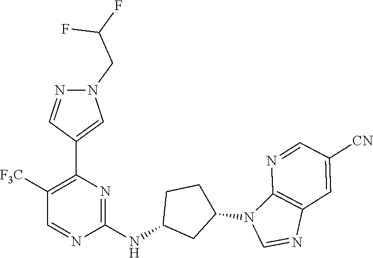 [SMILES: N#Cc1cnc2c(c1)ncn2[C@H]1CC[C@@H](Nc2ncc(C(F)(F)F)c(-c3cnn(CC(F)F)c3)n2)C1]

This compound was prepared according to the procedures described in Example 1, with tert-butyl ((1R,3S)-3-aminocyclopentyl)carbamate replacing tert-butyl ((1R,3S)-3-aminocyclohexyl)carbamate in Step 1. LCMS calculated for C₂₂H₁₉F₅N₉ (M+H)⁺: m/z=504.2; found: 504.2.

### Example 3. 2-(((1R,3S)-3-(1H-Benzo[d]imidazol-1-yl)cyclohexyl)amino)-4-(1-(2,2-difluoroethyl)-1H-pyrazol-4-yl)pyrimidine-5-carbonitrile

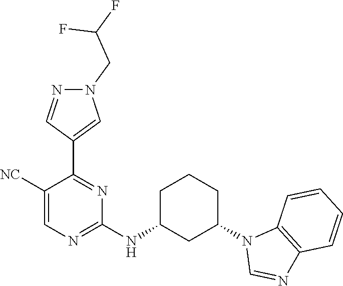 [SMILES: N#Cc1cnc(N[C@@H]2CCC[C@H](n3cnc4ccccc43)C2)nc1-c1cnn(CC(F)F)c1]

### Step 1. tert-Butyl ((1R,3S)-3-((2-nitrophenyl)amino)cyclohexyl)carbamate

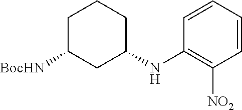 [SMILES: CC(C)(C)OC(=O)N[C@@H]1CCC[C@H](Nc2ccccc2[N+](=O)[O-])C1]

A mixture of tert-butyl ((1R,3S)-3-aminocyclohexyl)carbamate (227 mg, 1.06 mmol), 1-fluoro-2-nitrobenzene (136 mg, 0.96 mmol), and DIPEA (336 μL, 1.93 mmol) in EtOH (1 mL) was heated to 80° C. and stirred for 2 h. Upon completion, the reaction mixture was cooled to rt and concentrated in vacuo. The crude residue was purified by Biotage Isolera (hexanes/EtOAc, up to 100% EtOAc) to give the desired product. LCMS calculated for C₁₃H₁₈N₃O₄(M+H−C₄H₈)⁺: m/z=280.1; found: 280.1.

### Step 2. tert-Butyl ((1R,3S)-3-((2-aminophenyl)amino)cyclohexyl)carbamate

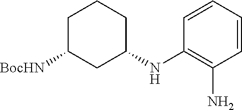 [SMILES: CC(C)(C)OC(=O)N[C@@H]1CCC[C@H](Nc2ccccc2N)C1]

A vial containing tert-butyl ((1R,3S)-3-((2-nitrophenyl)amino)cyclohexyl)carbamate (323 mg, 0.96 mmol) and Pd/C (5 wt % loading, Degussa type, 102 mg, 0.1 mmol) as a suspension in EtOH (10 mL) was evacuated and backfilled with an H₂ balloon three times, then stirred under H₂ balloon at rt for 2 h. The reaction mixture was filtered through Celite and the filtrate was concentrated in vacuo to give the desired product as a white solid. LCMS calculated for C₁₇H₂₈N₃O₂ (M+H)⁺: m/z=306.2; found: 306.2.

### Step 3. tert-Butyl ((1R,3S)-3-(1H-benzo[d]imidazol-1-yl)cyclohexyl)carbamate

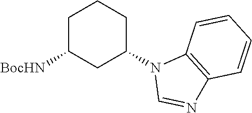 [SMILES: CC(C)(C)OC(=O)N[C@@H]1CCC[C@H](n2cnc3ccccc32)C1]

A vial containing tert-butyl ((1R,3S)-3-((2-aminophenyl)amino)cyclohexyl)carbamate (294 mg, 0.96 mmol) and trimethyl orthoformate (526 μL, 4.81 mmol) as a solution in toluene (10 mL) was sealed and heated to 90° C. for 2 h. Upon completion, the reaction mixture was concentrated in vacuo. The resulting residue was purified by Biotage Isolera (hexanes/EtOAc, up to 100% EtOAc) to give the desired product. LCMS calculated for C₁₈H₂₆N₃O₂ (M+H)⁺: m/z=316.2; found: 316.2.

### Step 4. (1R,3S)-3-(1H-Benzo[d]imidazol-1-yl)cyclohexan-1-amine

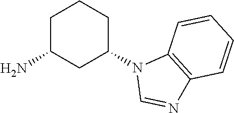 [SMILES: N[C@@H]1CCC[C@H](n2cnc3ccccc32)C1]

To a solution of tert-butyl ((1R,3S)-3-(1H-benzo[d]imidazol-1-yl)cyclohexyl)carbamate (302 mg, 0.96 mmol) in MeOH (3 mL) was added 4N HCl/dioxane (5 mL) and left to stir at rt for 1 h. Upon completion, the reaction mixture was concentrated in vacuo. The obtained crude product was used in the next step without further purification. LCMS calculated for C₁₃H₁₈N₃(M+H)⁺: m/z=216.1; found: 216.2.

### Step 5. 2-(((1R,3S)-3-(1H-Benzo[d]imidazol-1-yl)cyclohexyl)amino)-4-chloropyrimidine-5-carbonitrile

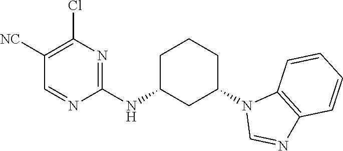 [SMILES: N#Cc1cnc(N[C@@H]2CCC[C@H](n3cnc4ccccc43)C2)nc1Cl]

To a solution of 2,4-dichloropyrimidine-5-carbonitrile (48 mg, 0.28 mmol) in t-butanol (1 mL) and 1,2-dichloroethane (1 mL) was added zinc(II) chloride (1.0 M in diethyl ether, 380 μL, 0.38 mmol). The solution was purged with nitrogen for 5 min, sealed and heated to 60° C. for 1 h, then cooled to rt. To this mixture was added a solution of (1R,3S)-3-(1H-benzo[d]imidazol-1-yl)cyclohexan-1-amine (51 mg, 0.28 mmol) in t-butanol (1.5 mL), 1,2-dichloroethane (1.5 mL) and DIPEA (75 L, 0.73 mmol). The reaction mixture was then reheated to 60° C. and stirred overnight. Upon completion, the solution was cooled to rt and the volatiles were removed in vacuo. The resulting residue was partitioned between CH₂Cl₂ (10 mL) and water (5 mL). The organic layer was washed with brine (5 mL), dried over sodium sulfate, filtered, and then concentrated in vacuo. The resulting residue was purified by Teledyne ISCO CombiFlash to give the desired product. LCMS calculated for C₁₈H₁₈ClN₆ (M+H)⁺: m/z=353.1; found: 353.1.

### Step 6. 2-(((1R,3S)-3-(1H-Benzo[d]imidazol-1-yl)cyclohexyl)amino)-4-(1-(2,2-difluoroethyl)-1H-pyrazol-4-yl)pyrimidine-5-carbonitrile

A mixture of 2-(((1R,3S)-3-(1H-benzo[d]imidazol-1-yl)cyclohexyl)amino)-4-chloropyrimidine-5-carbonitrile (10 mg, 0.03 mmol), 1-(2,2-difluoroethyl)-4-(4,4,5,5-tetramethyl-1,3,2-dioxaborolan-2-yl)-1H-pyrazole (10 mg, 0.04 mmol), and sodium carbonate (8 mg, 0.08 mmol) was dissolved in MeCN (970 μL) and water (190 μL). The solution was purged with nitrogen for 2 min, followed by addition of Pd(dppf)Cl₂ CH₂Cl₂ (2 mg, 2.6 mol). The vial was sealed and the reaction was stirred at 140° C. for 3 min before being cooled to rt. The solution was diluted with MeOH, filtered through a Silicycle SiliaPrep™ Thiol cartridge (Cat. #SPE-R51030B), and purified with prep-LCMS (XBridge C₁₈ column, eluting with a gradient of acetonitrile/water containing 0.1% TFA, at flow rate of 60 mL/min). LCMS calculated for C₂₃H₂₃F₂N₈ (M+H)⁺: m/z=449.2; found: 449.3.

### Example 4. 4-(1-(2,2-Difluoroethyl)-1H-pyrazol-4-yl)-5-(trifluoromethyl)-N-((1R,3S)-3-(6-(trifluoromethyl)-3H-imidazo[4,5-b]pyridin-3-yl)cyclohexyl)pyrimidin-2-amine

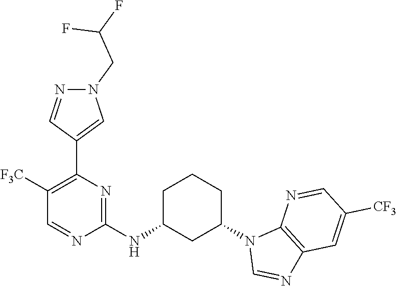 [SMILES: FC(F)Cn1cc(-c2nc(N[C@@H]3CCC[C@H](n4cnc5cc(C(F)(F)F)cnc54)C3)ncc2C(F)(F)F)cn1]

This compound was prepared according to the procedures described in Example 1, with 2-chloro-3-nitro-5-(trifluoromethyl)pyridine replacing 6-chloro-5-nitronicotinonitrile in Step 1. LCMS calculated for C₂₃H₂₁F₈N₈ (M+H)⁺: m/z=561.2; found: 561.3.

### Example 5. Methyl 3-((1S,3R)-3-((4-(1-(2,2-difluoroethyl)-1H-pyrazol-4-yl)-5-(trifluoromethyl)pyrimidin-2-yl)amino)cyclohexyl)-3H-imidazo[4,5-b]pyridine-5-carboxylate

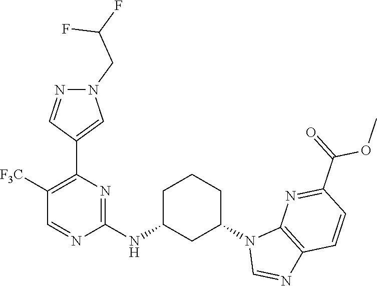 [SMILES: COC(=O)c1ccc2ncn([C@H]3CCC[C@@H](Nc4ncc(C(F)(F)F)c(-c5cnn(CC(F)F)c5)n4)C3)c2n1]

This compound was prepared according to the procedures described in Example 1, with methyl 6-chloro-5-nitropicolinate replacing 6-chloro-5-nitronicotinonitrile in Step 1. LCMS calculated for C₂₄H₂₄F₅N₈O₂(M+H)⁺: m/z=551.2; found: 551.3.

### Example 6. 4-(1-(2,2-Difluoroethyl)-1H-pyrazol-4-yl)-N-((1R,3S)-3-(5-methoxy-3H-imidazo[4,5-b]pyridin-3-yl)cyclohexyl)-5-(trifluoromethyl)pyrimidin-2-amine

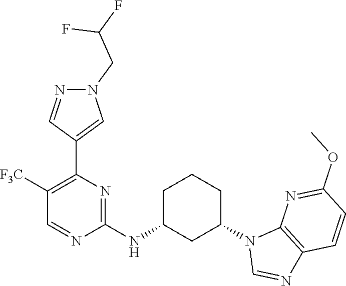 [SMILES: COc1ccc2ncn([C@H]3CCC[C@@H](Nc4ncc(C(F)(F)F)c(-c5cnn(CC(F)F)c5)n4)C3)c2n1]

This compound was prepared according to the procedures described in Example 1, with 2-chloro-6-methoxy-3-nitropyridine replacing 6-chloro-5-nitronicotinonitrile in Step 1. LCMS calculated for C₂₃H₂₄F₅N₈O (M+H)⁺: m/z=523.2; found: 523.3.

### Example 7. N-((1R,3S)-3-(1H-Imidazo[4,5-c]pyridin-1-yl)cyclohexyl)-4-(1-(2,2-difluoroethyl)-1H-pyrazol-4-yl)-5-(trifluoromethyl)pyrimidin-2-amine

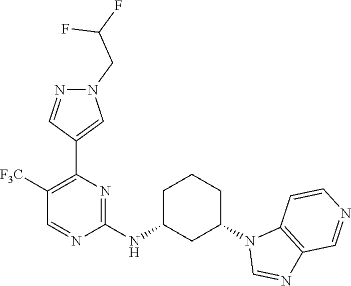 [SMILES: FC(F)Cn1cc(-c2nc(N[C@@H]3CCC[C@H](n4cnc5cnccc54)C3)ncc2C(F)(F)F)cn1]

This compound was prepared according to the procedures described in Example 1, with 4-chloro-3-nitropyridine replacing 6-chloro-5-nitronicotinonitrile in Step 1. LCMS calculated for C₂₂H₂₂F₅N₈ (M+H)⁺: m/z=493.2; found: 493.3.

### Example 8. 4-(1-(2,2-Difluoroethyl)-1H-pyrazol-4-yl)-2-(((1R,3S)-3-(7-methyl-3H-imidazo[4,5-b]pyridin-3-yl)cyclohexyl)amino)pyrimidine-5-carbonitrile

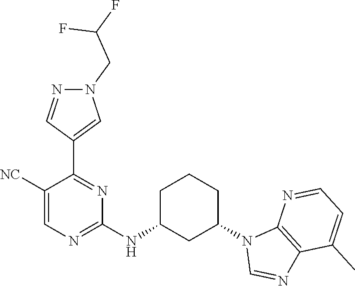 [SMILES: Cc1ccnc2c1ncn2[C@H]1CCC[C@@H](Nc2ncc(C#N)c(-c3cnn(CC(F)F)c3)n2)C1]

This compound was prepared according to the procedures described in Example 3, with 2-chloro-4-methyl-3-nitropyridine replacing 1-fluoro-2-nitrobenzene in Step 1. LCMS calculated for C₂₃H₂₄F₂N₉ (M+H)⁺: m/z=464.2; found: 464.3.

### Example 9. 3-((1S,3R)-3-((5-Cyano-4-(1-(2,2-difluoroethyl)-1H-pyrazol-4-yl)pyrimidin-2-yl)amino)cyclohexyl)-3H-imidazo[4,5-b]pyridine-6-carbonitrile

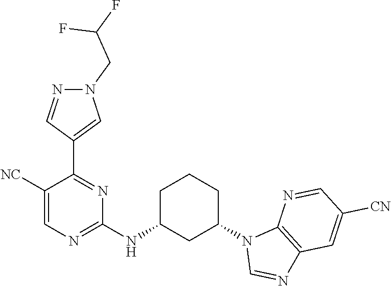 [SMILES: N#Cc1cnc2c(c1)ncn2[C@H]1CCC[C@@H](Nc2ncc(C#N)c(-c3cnn(CC(F)F)c3)n2)C1]

This compound was prepared according to the procedures described in Example 3, with 6-chloro-5-nitronicotinonitrile replacing 1-fluoro-2-nitrobenzene in Step 1. LCMS calculated for C₂₃H₂₁F₂N₁₀ (M+H)⁺: m/z=475.2; found: 475.3. ¹H NMR (500 MHz, DMSO, mixture of tautomers) δ 8.85-8.78 (m, 2H), 8.75-8.68 (m, 1.4H), 8.64 (d, J=11.8 Hz, 1H), 8.54 (s, 0.4H), 8.41 (s, 0.6H), 8.38-8.32 (m, 1H), 8.20 (s, 0.6H), 6.58-6.29 (m, 1H), 4.90-4.68 (m, 3H), 4.25-4.07 (m, 1H), 2.47-2.30 (m, 1H), 2.16-1.90 (m, 5H), 1.74-1.54 (m, 1H), 1.54-1.41 (m, 1H).

### Example 9A. Alternative Synthesis of 3-((1S,3R)-3-((5-Cyano-4-(1-(2,2-difluoroethyl)-1H-pyrazol-4-yl)pyrimidin-2-yl)amino)cyclohexyl)-3H-imidazo[4,5-b]pyridine-6-carbonitrile

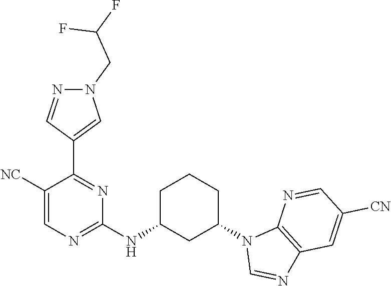 [SMILES: N#Cc1cnc2c(c1)ncn2[C@H]1CCC[C@@H](Nc2ncc(C#N)c(-c3cnn(CC(F)F)c3)n2)C1]

### Step 1. tert-Butyl ((1R,3S)-3-((5-cyano-3-nitropyridin-2-yl)amino)cyclohexyl)carbamate

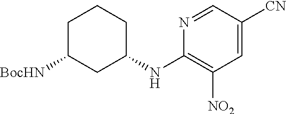 [SMILES: CC(C)(C)OC(=O)N[C@@H]1CCC[C@H](Nc2ncc(C#N)cc2[N+](=O)[O-])C1]

A mixture of tert-butyl ((1R,3S)-3-aminocyclohexyl)carbamate (1.17 g, 5.45 mmol), 6-chloro-5-nitronicotinonitrile (1.0 g, 5.4 mmol), and DIPEA (1.9 mL, 10.9 mmol) in EtOH (10 mL) was heated to 80° C. and stirred for 2 h. Upon completion, the reaction mixture was cooled to rt and concentrated in vacuo. The crude residue was purified by Biotage Isolera (CH₂Cl₂/MeOH, up to 10% MeOH) to give the desired product as yellow solid (1.7 g, 86% yield). LCMS calculated for C₁₃H₁₆N₅O₄ (M+H−C₄H₈)⁺: m/z=306.1; found: 306.1.

### Step 2. tert-Butyl ((1R,3S)-3-((3-amino-5-cyanopyridin-2-yl)amino)cyclohexyl)carbamate

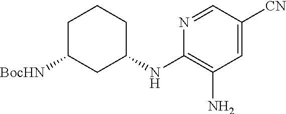 [SMILES: CC(C)(C)OC(=O)N[C@@H]1CCC[C@H](Nc2ncc(C#N)cc2N)C1]

A vial containing tert-butyl ((1R,3S)-3-((5-cyano-3-nitropyridin-2-yl)amino)cyclohexyl)carbamate (1.7 g, 4.7 mmol) and Pd/C (5 wt % loading, Degussa type, 501 mg, 0.47 mmol) as a suspension in EtOH (10 mL) was evacuated and backfilled with an H₂ balloon three times, then stirred under H₂ balloon at rt for 2 h. The reaction mixture was filtered through Celite and the filtrate was concentrated in vacuo to give the desired product as a white solid. LCMS calculated for C₁₇H₂₆N₅O₂ (M+H)⁺: m/z=332.2; found: 332.1.

### Step 3. tert-Butyl ((1R,3S)-3-(6-cyano-3H-imidazo[4,5-b]pyridin-3-yl)cyclohexyl)carbamate

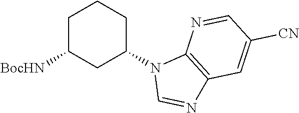 [SMILES: CC(C)(C)OC(=O)N[C@@H]1CCC[C@H](n2cnc3cc(C#N)cnc32)C1]

A vial containing tert-butyl ((1R,3S)-3-((3-amino-5-cyanopyridin-2-yl)amino)cyclohexyl)carbamate (1.4 g, 4.2 mmol), trimethyl orthoformate (2.3 mL, 21.1 mmol), and p-toluenesulfonic acid monohydrate (10 mg, 0.05 mmol) as a solution in toluene (10 mL) was sealed and heated to 90° C. for 2 h. Upon completion, the reaction mixture was concentrated in vacuo. The resulting residue was purified by Biotage Isolera (CH₂Cl₂/MeOH, up to 10% MeOH) to give the product as a colorless solid (1.1 g, 76% yield). LCMS calculated for C₁₈H₂₄N₅O₂ (M+H)⁺: m/z=342.2; found: 342.1.

### Step 4. 3-((1S,3R)-3-Aminocyclohexyl)-3H-imidazo[4,5-b]pyridine-6-carbonitrile

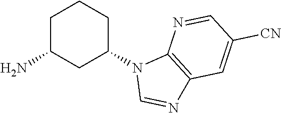 [SMILES: N#Cc1cnc2c(c1)ncn2[C@H]1CCC[C@@H](N)C1]

To a solution of tert-butyl ((1R,3S)-3-(6-cyano-3H-imidazo[4,5-b]pyridin-3-yl)cyclohexyl)carbamate (1.1 g, 3.2 mmol) in MeOH (3 mL) was added 4N HCl/dioxane (5 mL) and left to stir at rt for 1 h. Upon completion, the reaction mixture was concentrated in vacuo. The obtained crude product was used in the next step without further purification. LCMS calculated for C₁₃H₁₆N₅(M+H)⁺: m/z=242.1; found: 242.2.

### Step 5. 3-((1S,3R)-3-((4-Chloro-5-cyanopyrimidin-2-yl)amino)cyclohexyl)-3H-imidazo[4,5-b]pyridine-6-carbonitrile

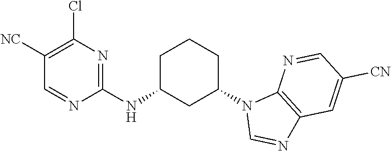 [SMILES: N#Cc1cnc2c(c1)ncn2[C@H]1CCC[C@@H](Nc2ncc(C#N)c(Cl)n2)C1]

To a solution of 2,4-dichloropyrimidine-5-carbonitrile (1.67 g, 9.6 mmol) in tert-BuOH (11 mL) and 1,2-dichloroethane (11 mL) was added zinc(II) chloride (1.0 M in diethyl ether, 26 mL, 13 mmol). The solution was purged with nitrogen for 5 min, sealed and heated to 60° C. for 1 h, then cooled to rt. To this mixture was added 3-((1S,3R)-3-aminocyclohexyl)-3H-imidazo[4,5-b]pyridine-6-carbonitrile (2.1 g, 8.7 mmol) in tert-BuOH (11 mL), 1,2-dichloroethane (11 mL) and DIPEA (6.1 mL, 35 mmol). The reaction mixture was then reheated to 60° C. and stirred overnight. Upon completion, the solution was cooled to rt and the volatiles were removed in vacuo. The resulting residue was partitioned between CH₂Cl₂ (100 mL) and water (50 mL). The organic layer was washed with brine (50 mL), dried over sodium sulfate, filtered, and then concentrated in vacuo. The resulting residue was purified by Teledyne ISCO CombiFlash to give the desired product. LCMS calculated for C₁₈H₁₆ClN₈ (M+H)⁺: m/z=379.1/381.1; found: 379.2/381.2.

### Step 6. 3-((1S,3R)-3-((5-Cyano-4-(1-(2,2-difluoroethyl)-1H-pyrazol-4-yl)pyrimidin-2-yl)amino)cyclohexyl)-3H-imidazo[4,5-b]pyridine-6-carbonitrile

A mixture of 1-((1S,3R)-3-((4-chloro-5-cyanopyrimidin-2-yl)amino)cyclohexyl)-1H-benzo[d]imidazole-5-carbonitrile (2.3 g, 6 mmol), 1-(2,2-difluoroethyl)-4-(4,4,5,5-tetramethyl-1,3,2-dioxaborolan-2-yl)-1H-pyrazole (1.71 g, 6.6 mmol), and sodium carbonate (1.9 g, 18 mmol) was dissolved in MeCN (24 mL) and water (4.5 mL). The solution was purged with nitrogen for 2 min, followed by addition of Pd(dppf)Cl₂ CH₂Cl₂ (492 mg, 0.6 mmol). The vial was sealed and the reaction was stirred at 140° C. for 5 min before being cooled to rt, diluted with CH₂C₁₂ (20 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₁F₂N₁₀ (M+H)⁺: m/z=475.2; found: 475.3. ¹H NMR (500 MHz, DMSO, mixture of tautomers) δ 8.85-8.78 (m, 2H), 8.75-8.68 (m, 1.4H), 8.64 (d, J=11.8 Hz, 1H), 8.54 (s, 0.4H), 8.41 (s, 0.6H), 8.38-8.32 (m, 1H), 8.20 (s, 0.6H), 6.58-6.29 (m, 1H), 4.90-4.68 (m, 3H), 4.25-4.07 (m, 1H), 2.47-2.30 (m, 1H), 2.16-1.90 (m, 5H), 1.74-1.54 (m, 1H), 1.54-1.41 (m, 1H).

### Example 10. N-((1R,3S)-3-(3H-Imidazo[4,5-c]pyridin-3-yl)cyclohexyl)-4-(1-(2,2-difluoroethyl)-1H-pyrazol-4-yl)-5-(trifluoromethyl)pyrimidin-2-amine

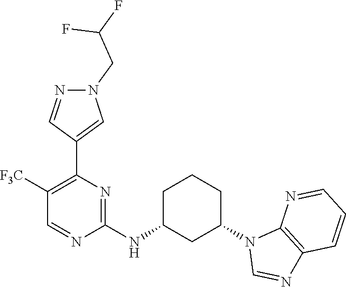 [SMILES: FC(F)Cn1cc(-c2nc(N[C@@H]3CCC[C@H](n4cnc5cccnc54)C3)ncc2C(F)(F)F)cn1]

This compound was prepared according to the procedures described in Example 1, with 3-fluoro-4-nitropyridine replacing 6-chloro-5-nitronicotinonitrile in Step 1. LCMS calculated for C₂₂H₂₂F₅N₈ (M+H)⁺: m/z=493.2; found: 493.3.

### Example 11. 2-(((1R,3S)-3-(3H-Imidazo[4,5-b]pyridin-3-yl)cyclohexyl)amino)-4-(1-(2,2-difluoroethyl)-1H-pyrazol-4-yl)pyrimidine-5-carbonitrile

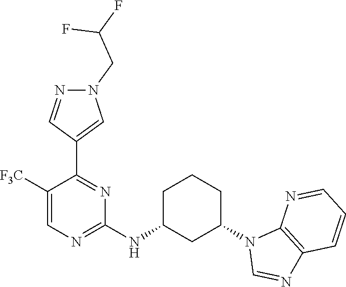 [SMILES: FC(F)Cn1cc(-c2nc(N[C@@H]3CCC[C@H](n4cnc5cccnc54)C3)ncc2C(F)(F)F)cn1]

This compound was prepared according to the procedures described in Example 3, with 2-chloro-3-nitropyridine replacing 1-fluoro-2-nitrobenzene in Step 1. LCMS calculated for C₂₂H₂₂F₂N₉ (M+H)⁺: m/z=450.2; found: 450.3.

### Example 12. N-((1R,3S)-3-(3H-Imidazo[4,5-b]pyridin-3-yl)cyclohexyl)-4-(1-(2,2-difluoroethyl)-1H-pyrazol-4-yl)-5-(trifluoromethyl)pyrimidin-2-amine

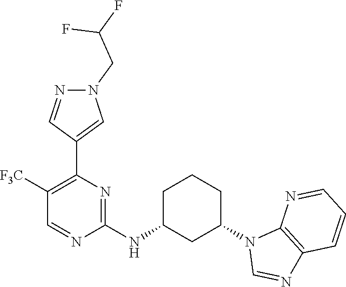 [SMILES: FC(F)Cn1cc(-c2nc(N[C@@H]3CCC[C@H](n4cnc5cccnc54)C3)ncc2C(F)(F)F)cn1]

This compound was prepared according to the procedures described in Example 1, with 2-chloro-3-nitropyridine replacing 6-chloro-5-nitronicotinonitrile in Step 1. LCMS calculated for C₂₂H₂₂F₅N₈ (M+H)⁺: m/z=493.2; found: 493.3. Updated ¹H NMR (600 MHz, DMSO, mixture of tautomers) δ 8.89 (s, 0.55H), 8.86 (s, 0.45H), 8.62 (s, 0.45H), 8.54 (s, 0.55H), 8.47 (dd, J=9.9, 4.7 Hz, 1H), 8.37 (s, 0.55H), 8.27 (s, 0.45H), 8.25 (s, 0.55H), 8.16 (dd, J=8.2, 3.0 Hz, 1H), 8.10 (d, J=7.7 Hz, 1H), 7.97 (s, 0.45H), 7.42-7.36 (m, 1H), 6.42 (tdt, J=54.6, 19.1, 3.7 Hz, 1H), 4.89-4.67 (m, 3H), 4.20-4.07 (m, 1H), 2.51 (s, 0.55H), 2.43-2.35 (m, 0.45H), 2.20-1.89 (m, 5H), 1.74-1.56 (m, 1H), 1.53-1.43 (m, 1H).

### Example 13. 1-((1S,3R)-3-((5-Cyano-4-(1-(2,2-difluoroethyl)-1H-pyrazol-4-yl)pyrimidin-2-yl)amino)cyclohexyl)-1H-benzo[d]imidazole-7-carbonitrile

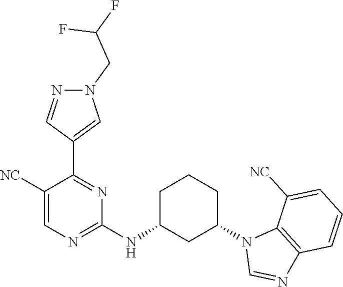 [SMILES: N#Cc1cnc(N[C@@H]2CCC[C@H](n3cnc4cccc(C#N)c43)C2)nc1-c1cnn(CC(F)F)c1]

### Step 1. tert-Butyl ((1R,3S)-3-((2-cyano-6-nitrophenyl)amino)cyclohexyl)carbamate

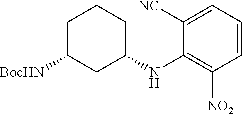 [SMILES: CC(C)(C)OC(=O)N[C@@H]1CCC[C@H](Nc2c(C#N)cccc2[N+](=O)[O-])C1]

In a glass vial, 2-fluoro-3-nitrobenzonitrile (80 mg, 0.48 mmol) and tert-butyl ((1R,3S)-3-aminocyclohexyl)carbamate (114 mg, 0.53 mmol) were dissolved in EtOH (1.6 mL). The vial was sealed with a screw-cap and the solution was stirred at 80° C. After 2 h, LCMS indicated consumption of the starting material. The solution was cooled to rt and concentrated in vacuo to provide tert-butyl ((1R,3S)-3-((2-cyano-6-nitrophenyl)amino)cyclohexyl)carbamate. The crude sample was used without further purification. LCMS calculated for C₁₈H₂₅N₄O₄ (M+H)⁺: m/z=361.2; found: 361.2.

### Step 2. tert-Butyl ((1R,3S)-3-((2-amino-6-cyanophenyl)amino)cyclohexyl)carbamate

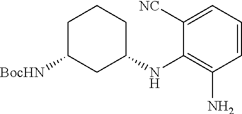 [SMILES: CC(C)(C)OC(=O)N[C@@H]1CCC[C@H](Nc2c(N)cccc2C#N)C1]

A glass vial was charged with crude tert-butyl ((1R,3S)-3-((2-cyano-6-nitrophenyl)amino)cyclohexyl)carbamate (see Step 1) followed by THF (810 μL), MeOH (810 μL), and water (810 μL). To this solution was added iron powder (135 mg, 2.41 mmol) and ammonium chloride (155 mg, 2.90 mmol). The vial was sealed with a screw-cap and the slurry was stirred at 60° C. After 2 h, LCMS indicated consumption of the starting material. The reaction mixture was cooled to rt, diluted with CH₂Cl₂ (5 mL), and filtered through a plug of Celite. The filtrate was concentrated in vacuo to provide tert-butyl ((1R,3S)-3-((2-amino-6-cyanophenyl)amino)cyclohexyl)carbamate. The crude sample was used without further purification. LCMS calculated for C₁₈H₂₇N₄O₂ (M+H)⁺: m/z=331.2; found: 331.1.

### Step 3. tert-Butyl ((1R,3S)-3-(7-cyano-1H-benzo[d]imidazol-1-yl)cyclohexyl)carbamate

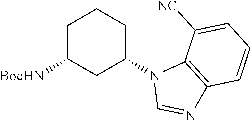 [SMILES: CC(C)(C)OC(=O)N[C@@H]1CCC[C@H](n2cnc3cccc(C#N)c32)C1]

A glass vial was charged with tert-butyl ((1R,3S)-3-((2-amino-6-cyanophenyl)amino)cyclohexyl)carbamate (see Step 2) followed by toluene (2.4 mL). Next, trimethyl orthoformate (320 μL, 2.9 mmol) was added by syringe, the vial was sealed, and the reaction was stirred at 120° C. LCMS indicated consumption of the starting material after 18 h. The solution was cooled to rt and concentrated in vacuo. The resulting residue was purified by Teledyne ISCO CombiFlash (hexanes/EtOAc, up to 100% EtOAc) to give tert-butyl ((1R,3S)-3-(7-cyano-1H-benzo[d]imidazol-1-yl)cyclohexyl)carbamate (116 mg, 70% yield over 3 steps). LCMS calculated for Cl₉H₂₅N₄O₂ (M+H)⁺: m/z=341.2; found: 341.1.

### Step 4. 1-((1S,3R)-3-((4-Chloro-5-cyanopyrimidin-2-yl)amino)cyclohexyl)-1H-benzo[d]imidazole-7-carbonitrile

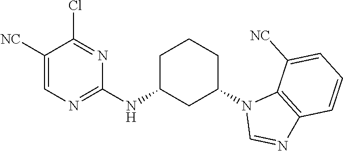 [SMILES: N#Cc1cnc(N[C@@H]2CCC[C@H](n3cnc4cccc(C#N)c43)C2)nc1Cl]

A sample of tert-butyl ((1R,3S)-3-(7-cyano-1H-benzo[d]imidazol-1-yl)cyclohexyl)carbamate (116 mg, 0.34 mmol) was dissolved in a solution of hydrochloric acid (4 M in dioxane, 1.3 mL, 5.1 mmol) and methanol (500 μL). The vial was sealed and the reaction was stirred at rt for 30 min, at which point LCMS indicated consumption of the starting material to form 1-((1S,3R)-3-aminocyclohexyl)-1H-benzo[d]imidazole-7-carbonitrile. The solution was subsequently concentrated in vacuo and the residue was redissolved in tert-butanol (1.5 mL) and 1,2-dichloroethane (1.5 mL). In a separate vial, 2,4-dichloropyrimidine-5-carbonitrile (48 mg, 0.28 mmol) was dissolved in tert-butanol (1 mL) and 1,2-dichloroethane (1 mL). To this mixture, zinc chloride (1.0 M in diethyl ether, 380 μL, 0.38 mmol) was added by syringe. The solution was purged with nitrogen for 5 min, then the vial was sealed. This reaction was stirred at 60° C. for 1 h, then cooled to rt, followed by the addition of 1-((1S,3R)-3-aminocyclohexyl)-1H-benzo[d]imidazole-7-carbonitrile solution and N,N-diisopropylethylamine (75 μL, 0.73 mmol), the reaction mixture was reheated to 60° C. and stirred for 22 h. After this time, LCMS indicated consumption of the starting material. The solution was cooled to rt and concentrated in vacuo. The resulting residue was partitioned between CH₂Cl₂ (10 mL) and water (5 mL). The organic layer was washed with brine (5 mL), dried over sodium sulfate, filtered, and concentrated in vacuo. The resulting residue was purified by Teledyne ISCO CombiFlash (hexanes/EtOAc, up to 100% EtOAc) to give 1-((1S,3R)-3-((4-chloro-5-cyanopyrimidin-2-yl)amino)cyclohexyl)-1H-benzo[d]imidazole-7-carbonitrile (10 mg, 0.03 mmol, 10% yield). LCMS calculated for C₁₉H₁₇ClN₇ (M+H)⁺: m/z=378.1; found: 378.1.

### Step 5. 1-((1S,3R)-3-((5-Cyano-4-(1-(2,2-difluoroethyl)-1H-pyrazol-4-yl)pyrimidin-2-yl)amino)cyclohexyl)-1H-benzo[d]imidazole-7-carbonitrile

A mixture of 1-((1S,3R)-3-((4-chloro-5-cyanopyrimidin-2-yl)amino)cyclohexyl)-1H-benzo[d]imidazole-7-carbonitrile (10 mg, 0.03 mmol), 1-(2,2-difluoroethyl)-4-(4,4,5,5-tetramethyl-1,3,2-dioxaborolan-2-yl)-1H-pyrazole (10 mg, 0.04 mmol), and sodium carbonate (8 mg, 0.08 mmol) was suspended in MeCN (970 μL) and water (190 μL). The solution was purged with nitrogen for 2 min, followed by addition of Pd(dppf)Cl₂ CH₂Cl₂ (2 mg, 2.6 mol). The vial was sealed and the reaction was stirred at 140° C. for 3 min before being cooled to rt. The solution was diluted with MeOH and filtered through a Silicycle SiliaPrep™ Thiol cartridge (Cat. #SPE-R51030B). The filtrate was then diluted in MeCN and purified by prep-LCMS (XBridge C₁₈ column, eluting with a gradient of acetonitrile/water containing 0.1% TFA, at flow rate of 60 mL/min). LCMS calculated for C₂₄H₂₂F₂N₉ (M+H)⁺: m/z=474.2; found: 474.3.

### Example 13A. Alternative Synthesis of 1-((1S,3R)-3-((5-Cyano-4-(1-(2,2-difluoroethyl)-1H-pyrazol-4-yl)pyrimidin-2-yl)amino)cyclohexyl)-1H-benzo[d]imidazole-7-carbonitrile

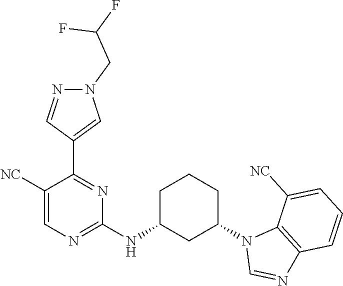 [SMILES: N#Cc1cnc(N[C@@H]2CCC[C@H](n3cnc4cccc(C#N)c43)C2)nc1-c1cnn(CC(F)F)c1]

This compound was prepared according to the procedures described in Example 9A, with 2-fluoro-3-nitrobenzonitrile replacing 6-chloro-5-nitronicotinonitrile in Step 1. LCMS calculated for C₂₄H₂₂F₂N₉ (M+H)⁺: m/z=474.2; found: 474.3.

### Example 14. 3-((1S,3R)-3-((5-Chloro-4-(1-(2,2-difluoroethyl)-1H-pyrazol-4-yl)pyrimidin-2-yl)amino)cyclohexyl)-3H-imidazo[4,5-b]pyridine-6-carbonitrile

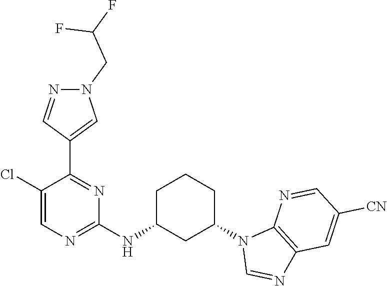 [SMILES: N#Cc1cnc2c(c1)ncn2[C@H]1CCC[C@@H](Nc2ncc(Cl)c(-c3cnn(CC(F)F)c3)n2)C1]

### Step 1. 2,5-Dichloro-4-(1-(2,2-difluoroethyl)-1H-pyrazol-4-yl)pyrimidine

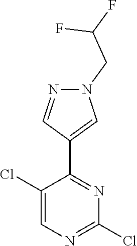 [SMILES: FC(F)Cn1cc(-c2nc(Cl)ncc2Cl)cn1]

A mixture of 2,4,5-trichloropyrimidine (178 μL, 1.55 mmol), 1-(2,2-difluoroethyl)-4-(4,4,5,5-tetramethyl-1,3,2-dioxaborolan-2-yl)-1H-pyrazole (400 mg, 1.55 mmol), and sodium carbonate (246 mg, 2.33 mmol) was dissolved in MeCN (7 mL) and water (700 μL). The solution was purged with nitrogen for 2 min, followed by addition of Pd(dppf)Cl₂ CH₂Cl₂ (127 mg, 155 mol). The vial was sealed and the reaction was stirred at 80° C. After 2 h, LCMS indicated consumption of the starting material. The solution was cooled to rt and filtered through a pad of Celite. The filtrate was concentrated in vacuo and the resulting residue was purified by Teledyne ISCO CombiFlash (hexanes/EtOAc, up to 60% EtOAc) to give 2,5-dichloro-4-(1-(2,2-difluoroethyl)-1H-pyrazol-4-yl)pyrimidine (317 mg, 73% yield). LCMS calculated for C₉H₇Cl₂F₂N₄ (M+H)⁺: m/z=279.0/281.0; found: 279.0/281.0.

### Step 2. 3-((1S,3R)-3-((5-Chloro-4-(1-(2,2-difluoroethyl)-1H-pyrazol-4-yl)pyrimidin-2-yl)amino)cyclohexyl)-3H-imidazo[4,5-b]pyridine-6-carbonitrile

A sample of tert-butyl ((1R,3S)-3-(6-cyano-3H-imidazo[4,5-b]pyridin-3-yl)cyclohexyl)carbamate (336 mg, 0.99 mmol, Example 9A, Step 3) was dissolved in a solution of hydrochloric acid (4 N in dioxane, 3.7 mL, 15 mmol) and MeOH (1.5 mL). The vial was sealed and the reaction was stirred at rt for 30 min, at which point LCMS indicated consumption of the starting material and deprotection to form 3-((1S,3R)-3-aminocyclohexyl)-3H-imidazo[4,5-b]pyridine-6-carbonitrile. The solution was subsequently concentrated in vacuo. To the residue was added 2,5-dichloro-4-(1-(2,2-difluoroethyl)-1H-pyrazol-4-yl)pyrimidine (262 mg, 0.99 mmol) and potassium fluoride (234 mg, 4.03 mmol). The solids were suspended in 1-butanol (3.2 mL). The vial was sealed and the reaction mixture was stirred at 120° C. After 19 h, LCMS indicated consumption of the starting material. The solution was cooled to rt and concentrated in vacuo. A sample of the resulting crude residue was dissolved in MeCN and purified by prep-LCMS (XBridge C₁₈ column, eluting with a gradient of acetonitrile/water containing 0.1% TFA, at flow rate of 60 mL/min). LCMS calculated for C₂₂H₂₁ClF₂N₉(M+H)⁺: m/z=484.2; found: 484.2.

### Example 15. 1-((1S,3R)-3-((4-(1-(2,2-Difluoroethyl)-1H-pyrazol-4-yl)-5-(trifluoromethyl)pyrimidin-2-yl)amino)cyclohexyl)-1H-pyrrolo[2,3-b]pyridine-5-carbonitrile

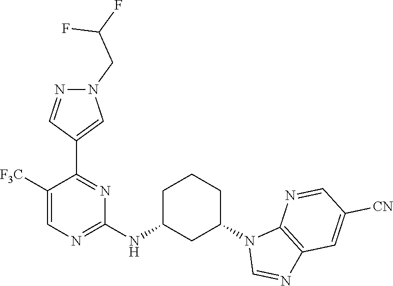 [SMILES: N#Cc1cnc2c(c1)ncn2[C@H]1CCC[C@@H](Nc2ncc(C(F)(F)F)c(-c3cnn(CC(F)F)c3)n2)C1]

### Step 1. tert-Butyl ((1R,3S)-3-(5-cyano-1H-pyrrolo[2,3-b]pyridin-1-yl)cyclohexyl)carbamate

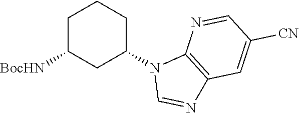 [SMILES: CC(C)(C)OC(=O)N[C@@H]1CCC[C@H](n2cnc3cc(C#N)cnc32)C1]

In a dry glass vial, 1H-pyrrolo[2,3-b]pyridine-5-carbonitrile (44 mg, 0.31 mmol) and tert-butyl ((1R,3R)-3-hydroxycyclohexyl)carbamate (100 mg, 0.46 mmol) were dissolved in anhydrous toluene (1.2 mL) followed by the addition of (tributylphosphoranylidene)acetonitrile (121 μL, 0.46 mmol). The vial was sealed and the solution was stirred at 100° C. After 3 h, LCMS indicated consumption of the starting material. The solution was cooled to rt and concentrated in vacuo. The resulting residue was purified by Teledyne ISCO CombiFlash (hexanes/EtOAc, up to 80% EtOAc) to give tert-butyl ((1R,3S)-3-(5-cyano-1H-pyrrolo[2,3-b]pyridin-1-yl)cyclohexyl)carbamate (14 mg, 0.04 mmol, 13% yield). LCMS calculated for C₁₅H₁₇N₄O₂ (M+H−C₄H₈: m/z=285.1; found: 285.1.

### Step 2. 2-Chloro-4-(1-(2,2-difluoroethyl)-1H-pyrazol-4-yl)-5-(trifluoromethyl)pyrimidine

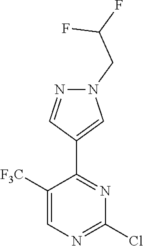 [SMILES: FC(F)Cn1cc(-c2nc(Cl)ncc2C(F)(F)F)cn1]

A mixture of 2,4-dichloro-5-(trifluoromethyl)pyrimidine (314 μL, 2.33 mmol), 1-(2,2-difluoroethyl)-4-(4,4,5,5-tetramethyl-1,3,2-dioxaborolan-2-yl)-1H-pyrazole (500 mg, 1.94 mmol), and sodium carbonate (411 mg, 3.87 mmol) was dissolved in MeCN (8 mL) and water (1.6 mL). The solution was purged with nitrogen for 2 min, followed by addition of Pd(dppf)Cl₂ CH₂Cl₂ (158 mg, 0.19 mmol). The vial was sealed and the reaction was stirred at 80° C. After 2 h, LCMS indicated consumption of the starting material. The solution was cooled to rt and filtered through a pad of Celite. The filtrate was concentrated in vacuo and the resulting residue was purified by Teledyne ISCO CombiFlash (hexanes/EtOAc, up to 70% EtOAc) to give 2-chloro-4-(1-(2,2-difluoroethyl)-1H-pyrazol-4-yl)-5-(trifluoromethyl)pyrimidine (334 mg, 55% yield). LCMS calculated for C₁₀H₇ClF₅N₄(M+H)⁺: m/z=313.0; found: 313.0.

### Step 3. 1-((1S,3R)-3-((4-(1-(2,2-Difluoroethyl)-1H-pyrazol-4-yl)-5-(trifluoromethyl)pyrimidin-2-yl)amino)cyclohexyl)-1H-pyrrolo[2,3-b]pyridine-5-carbonitrile

A sample of tert-butyl ((1R,3S)-3-(5-cyano-1H-pyrrolo[2,3-b]pyridin-1-yl)cyclohexyl)carbamate (14 mg, 0.04 mmol) was dissolved in a solution of hydrochloric acid (4 N in dioxane, 250 μL, 0.98 mmol) in a vial. The vial was sealed and the reaction was stirred at rt for 50 min, at which point LCMS indicated consumption of the starting material and deprotection to form 1-((1S,3R)-3-aminocyclohexyl)-1H-pyrrolo[2,3-b]pyridine-5-carbonitrile. The solution was subsequently concentrated in vacuo. To the residue was added 2-chloro-4-(1-(2,2-difluoroethyl)-1H-pyrazol-4-yl)-5-(trifluoromethyl)pyrimidine (15 mg, 0.05 mmol). The solids were dissolved in EtOH (960 μL) and N,N-diisopropylethylamine (29 L, 0.17 mmol) was then added. The vial was sealed and the reaction mixture was stirred at 60° C. After 2 h, LCMS indicated consumption of the starting material. The solution was cooled to rt and concentrated in vacuo. The residue was then dissolved in MeCN and purified by prep-LCMS (XBridge C₁₈ column, eluting with a gradient of acetonitrile/water containing 0.1% TFA, at flow rate of 60 mL/min). LCMS calculated for C₂₄H₂₂F₅N₈ (M+H)⁺: m/z=517.2; found: 517.2.

### Example 16. 1-((1S,3R)-3-((4-(1-(2,2-Difluoroethyl)-1H-pyrazol-4-yl)-5-(trifluoromethyl)pyrimidin-2-yl)amino)cyclohexyl)-1H-indole-5-carbonitrile

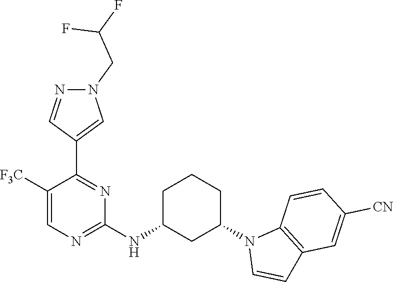 [SMILES: N#Cc1ccc2c(ccn2[C@H]2CCC[C@@H](Nc3ncc(C(F)(F)F)c(-c4cnn(CC(F)F)c4)n3)C2)c1]

Step 1. tert-Butyl ((1R,3S)-3-(5-cyano-1H-indol-1-yl)cyclohexyl)carbamate

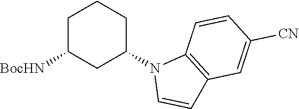 [SMILES: CC(C)(C)OC(=O)N[C@@H]1CCC[C@H](n2ccc3cc(C#N)ccc32)C1]

In a dry glass vial, 1H-indole-5-carbonitrile (44 mg, 0.31 mmol) and tert-butyl ((1R,3R)-3-hydroxycyclohexyl)carbamate (100 mg, 0.46 mmol) were dissolved in anhydrous toluene (1.2 mL). Following the addition of (tributylphosphoranylidene)acetonitrile (122 L, 0.46 mmol), the vial was sealed and stirred at 100° C. After 3 h, LCMS indicated consumption of the starting material. The solution was cooled to rt and concentrated in vacuo. The resulting residue was purified by Teledyne ISCO CombiFlash (hexanes/EtOAc, up to 60% EtOAc) to give tert-butyl ((1R,3S)-3-(5-cyano-1H-indol-1-yl)cyclohexyl)carbamate (5 mg, 0.01 mmol, 4% yield). LCMS calculated for C₁₆H₁₈N₃O₂ (M+H−C₄H₈)⁺: m/z=284.1; found: 284.2.

### Step 2. 1-((1S,3R)-3-((4-(1-(2,2-Difluoroethyl)-1H-pyrazol-4-yl)-5-(trifluoromethyl)pyrimidin-2-yl)amino)cyclohexyl)-1H-indole-5-carbonitrile

A sample of tert-butyl ((1R,3S)-3-(5-cyano-1H-indol-1-yl)cyclohexyl)carbamate (5 mg, 0.01 mmol) was dissolved in a solution of hydrochloric acid (4 M in dioxane, 80 μL, 0.32 mmol). The vial was sealed and the reaction was stirred at rt for 50 min, at which point LCMS indicated consumption of the starting material and deprotection to form 1-((1S,3R)-3-aminocyclohexyl)-1H-indole-5-carbonitrile. The solution was subsequently concentrated in vacuo. To the residue was added 2-chloro-4-(1-(2,2-difluoroethyl)-1H-pyrazol-4-yl)-5-(trifluoromethyl)pyrimidine (15 mg, 0.05 mmol, Example 15, Step 2). The solids were dissolved in EtOH (960 μL) followed by the addition of N,N-diisopropylethylamine (29 μL, 0.17 mmol). The vial was sealed and the reaction mixture was stirred at 60° C. After 2 h, LCMS indicated consumption of the starting material. The solution was cooled to rt and concentrated in vacuo. The crude residue was then dissolved in MeCN and purified by prep-LCMS (XBridge C₁₈ column, eluting with a gradient of acetonitrile/water containing 0.1% TFA, at flow rate of 60 mL/min). LCMS calculated for C₂₅H₂₃F₅N₇ (M+H)⁺: m/z=516.2; found: 516.2.

### Example 17. 2-(((1R,3S)-3-(3H-Imidazo[4,5-b]pyridin-3-yl)cyclohexyl)amino)-4-(1,5-dimethyl-1H-pyrazol-4-yl)pyrimidine-5-carbonitrile

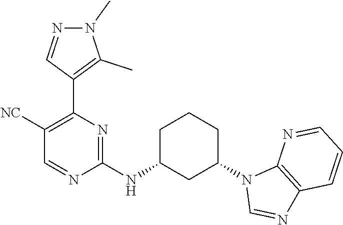 [SMILES: Cc1c(-c2nc(N[C@@H]3CCC[C@H](n4cnc5cccnc54)C3)ncc2C#N)cnn1C]

### Step 1. tert-Butyl ((1R,3S)-3-((3-nitropyridin-2-yl)amino)cyclohexyl)carbamate

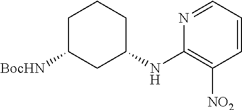 [SMILES: CC(C)(C)OC(=O)N[C@@H]1CCC[C@H](Nc2ncccc2[N+](=O)[O-])C1]

A mixture of tert-butyl ((1R,3S)-3-aminocyclohexyl)carbamate (3 g, 14.1 mmol), 2-chloro-3-nitropyridine (2 g, 12.6 mmol), and DIPEA (3.3 mL, 19.0 mmol) in EtOH (40 mL) was heated to 80° C. and stirred for 2 h. Upon completion, the reaction mixture was cooled to rt and concentrated in vacuo. The crude residue was taken on directly to the next reaction without further purification. LCMS calculated for C₁₂H₁₇N₄O₄ (M+H−C₄H₈)⁺: m/z=281.1; found: 281.1.

### Step 2. tert-Butyl ((1R,3S)-3-((3-aminopyridin-2-yl)amino)cyclohexyl)carbamate

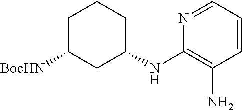 [SMILES: CC(C)(C)OC(=O)N[C@@H]1CCC[C@H](Nc2ncccc2N)C1]

To a flask containing tert-butyl ((1R,3S)-3-((3-nitropyridin-2-yl)amino)cyclohexyl)carbamate (4.2 g, 12.6 mmol) as a solution in THE (21 mL), MeOH (21 mL), and water (21 mL) was added iron powder (3.5 g, 63 mmol) and ammonium chloride (4 g, 76 mmol). The reaction mixture was heated to 60° C. for 2 h. After cooling to rt, the reaction mixture was diluted with CH₂Cl₂(10 mL) and filtered over Celite. The filtrate was concentrated in vacuo and the resulting crude residue was used in the next step with no further purification. LCMS calculated for C₁₆H₂₇N₄O₂ (M+H)⁺: m/z=307.2; found: 307.2.

### Step 3. (1R,3S)-3-(3H-Imidazo[4,5-b]pyridin-3-yl)cyclohexan-1-amine

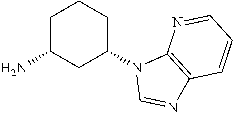 [SMILES: N[C@@H]1CCC[C@H](n2cnc3cccnc32)C1]

A flask containing tert-butyl ((1R,3S)-3-((3-aminopyridin-2-yl)amino)cyclohexyl)carbamate (3.68 g, 12 mmol), and trimethyl orthoformate (7.9 mL, 72.1 mmol) as a solution in toluene (60 mL) was heated to 120° C. overnight. Upon completion, the reaction mixture was concentrated in vacuo. The resulting residue was purified by Teledyne ISCO CombiFlash (CH₂Cl₂/MeOH, up to 10% MeOH). The purified product was then treated with 4N HCl/dioxane (6 mL) and left to stir at rt for 1 h before removing the volatiles in vacuo. The resulting crude residue was used directly in the next reaction without further purification. LCMS calculated for C₁₂H₁₇N₄ (M+H)⁺: m/z=217.1; found: 217.1.

### Step 4. 2-(((1R,3S)-3-(3H-Imidazo[4,5-b]pyridin-3-yl)cyclohexyl)amino)-4-chloropyrimidine-5-carbonitrile

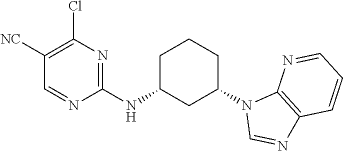 [SMILES: N#Cc1cnc(N[C@@H]2CCC[C@H](n3cnc4cccnc43)C2)nc1Cl]

To a solution of 2,4-dichloropyrimidine-5-carbonitrile (885 mg, 5.1 mmol) in t-butanol (5 mL) and 1,2-dichloroethane (5 mL) was added zinc(II) chloride (1.0 M in diethyl ether, 6.9 mL, 6.9 mmol). The solution was purged with nitrogen for 5 min, sealed and heated to 60° C. for 1 h, then cooled to rt. To this mixture was added a solution of (1R,3S)-3-(3H-imidazo[4,5-b]pyridin-3-yl)cyclohexan-1-amine (1 g, 4.6 mmol) in t-butanol (5 mL), 1,2-dichloroethane (5 mL) and DIPEA (2.4 mL, 13.9 mmol). The reaction mixture was then reheated to 60° C. and stirred for 3 h. Upon completion, the solution was cooled to rt and the volatiles were removed in vacuo. The resulting residue was purified by Teledyne ISCO CombiFlash (CH₂Cl₂/MeOH, up to 10% MeOH) to give the desired product. LCMS calculated for C₁₇H₁₇ClN₇ (M+H)⁺: m/z=354.1/356.1; found: 354.1/356.1.

### Step 5. 2-(((1R,3S)-3-(3H-Imidazo[4,5-b]pyridin-3-yl)cyclohexyl)amino)-4-(1,5-dimethyl-1H-pyrazol-4-yl)pyrimidine-5-carbonitrile

A vial containing 2-(((1R,3S)-3-(3H-imidazo[4,5-b]pyridin-3-yl)cyclohexyl)amino)-4-chloropyrimidine-5-carbonitrile (10 mg, 0.03 mmol), 1,5-dimethyl-4-(4,4,5,5-tetramethyl-1,3,2-dioxaborolan-2-yl)-1H-pyrazole (7 mg, 0.03 mmol), sodium carbonate (9 mg, 0.08 mmol), and Pd(dppf)Cl₂ CH₂Cl₂ (2 mg, 2.8 μmol) was evacuated and backfilled with nitrogen three times, followed by the addition of MeCN (1 mL) and water (200 μL). The vial was sealed and heated to 140° C. for 3 min. After cooling to rt, the mixture was filtered through a Silicycle SiliaPrep™ Thiol cartridge (Cat. #SPE-R51030B), washing with MeCN, and purified with prep-LCMS (XBridge C₁₈ column, eluting with a gradient of acetonitrile/water containing 0.1% TFA, at flow rate of 60 mL/min). LCMS calculated for C₂₂H₂₄N₉ (M+H)⁺: m/z=414.2; found: 414.1.

### Example 18. 2-(((1R,3S)-3-(3H-Imidazo[4,5-b]pyridin-3-yl)cyclohexyl)amino)-4-(5,6-dihydro-4H-pyrrolo[1,2-b]pyrazol-3-yl)pyrimidine-5-carbonitrile

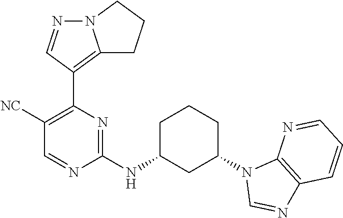 [SMILES: N#Cc1cnc(N[C@@H]2CCC[C@H](n3cnc4cccnc43)C2)nc1-c1cnn2c1CCC2]

This compound was prepared according to the procedures described in Example 17, with 3-(4,4,5,5-tetramethyl-1,3,2-dioxaborolan-2-yl)-5,6-dihydro-4H-pyrrolo[1,2-b]pyrazole replacing 1,5-dimethyl-4-(4,4,5,5-tetramethyl-1,3,2-dioxaborolan-2-yl)-1H-pyrazole in Step 5. LCMS calculated for C₂₃H₂₄N₉(M+H)⁺: m/z=426.2; found: 426.2.

### Example 19. 2-(((1R,3S)-3-(3H-Imidazo[4,5-b]pyridin-3-yl)cyclohexyl)amino)-4-(1,3-dimethyl-1H-pyrazol-4-yl)pyrimidine-5-carbonitrile

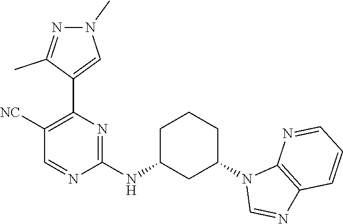 [SMILES: Cc1nn(C)cc1-c1nc(N[C@@H]2CCC[C@H](n3cnc4cccnc43)C2)ncc1C#N]

This compound was prepared according to the procedures described in Example 17, with 1,3-dimethyl-4-(4,4,5,5-tetramethyl-1,3,2-dioxaborolan-2-yl)-1H-pyrazole replacing 1,5-dimethyl-4-(4,4,5,5-tetramethyl-1,3,2-dioxaborolan-2-yl)-1H-pyrazole in Step 5. LCMS calculated for C₂₂H₂₄N₉(M+H)⁺: m/z=414.2; found: 414.1. Updated ¹H NMR (500 MHz, DMSO, mixture of tautomers) δ 9.01-8.90 (m, 1H), 8.68 (s, 0.3H), 8.59 (s, 0.7H), 8.51-8.46 (m, 1H), 8.44 (s, 0.7H), 8.38-8.30 (m, 1H), 8.24-8.15 (m, 1.3H), 7.46-7.38 (m, 1H), 4.81-4.70 (m, 1H), 4.18-4.05 (m, 1H), 3.90-3.80 (m, 3H), 2.54 (s, 2H), 2.48-2.36 (m, 2H), 2.21-1.92 (m, 5H), 1.66-1.52 (m, 1H), 1.52-1.41 (m, 1H).

### Example 20. 2-(((1R,3S)-3-(3H-Imidazo[4,5-b]pyridin-3-yl)cyclohexyl)amino)-4-(5-cyano-1-methyl-1H-pyrazol-4-yl)pyrimidine-5-carbonitrile

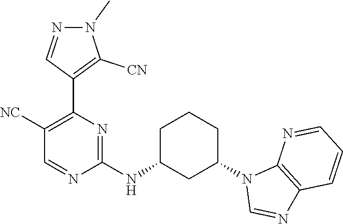 [SMILES: Cn1ncc(-c2nc(N[C@@H]3CCC[C@H](n4cnc5cccnc54)C3)ncc2C#N)c1C#N]

This compound was prepared according to the procedures described in Example 17, with 1-methyl-4-(4,4,5,5-tetramethyl-1,3,2-dioxaborolan-2-yl)-1H-pyrazole-5-carbonitrile replacing 1,5-dimethyl-4-(4,4,5,5-tetramethyl-1,3,2-dioxaborolan-2-yl)-1H-pyrazole in Step 5. LCMS calculated for C₂₂H₂₁N₁₀ (M+H)⁺: m/z=425.2; found: 425.1. Updated ¹H NMR (500 MHz, DMSO-d₆, mixture of tautomers) δ 8.87-8.78 (m, 1H), 8.75 (s, 1H), 8.64 (d, J=8.2 Hz, 1H), 8.45 (dd, J=4.6, 1.4 Hz, 1H), 8.43-8.38 (m, 0.9H), 8.30 (s, 0.1H), 8.15 (dd, J=8.1, 1.5 Hz, 1H), 7.39 (dd, J=8.1, 4.7 Hz, 1H), 471, (tt, J=12.3, 3.9 Hz, 1H), 4.39-4.29 (m, 0.9H), 4.24-4.16 (m, 0.1H), 4.15-4.07 (m, 3H), 2.42 (d, J=11.7 Hz, 0.9H), 2.36 (d, J=11.5 Hz, 0.1H), 2.26-1.85 (m, 5H), 1.67-1.53 (m, 1H), 1.52-1.40 (m, 1H).

### Example 21. 2-(((1R,3S)-3-(3H-Imidazo[4,5-b]pyridin-3-yl)cyclohexyl)amino)-4-(3-cyclopropyl-1H-pyrazol-4-yl)pyrimidine-5-carbonitrile

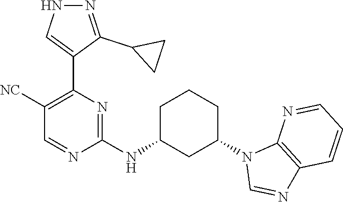 [SMILES: N#Cc1cnc(N[C@@H]2CCC[C@H](n3cnc4cccnc43)C2)nc1-c1c[nH]nc1C1CC1]

This compound was prepared according to the procedures described in Example 17, with (1-(tert-butoxycarbonyl)-3-cyclopropyl-1H-pyrazol-4-yl)boronic acid replacing 1,5-dimethyl-4-(4,4,5,5-tetramethyl-1,3,2-dioxaborolan-2-yl)-1H-pyrazole in Step 5. LCMS calculated for C₂₃H₂₄N₉(M+H)⁺: m/z=426.2; found: 426.2.

### Example 22. 2-(((1R,3S)-3-(3H-Imidazo[4,5-b]pyridin-3-yl)cyclohexyl)amino)-4-(1-methyl-1H-pyrazol-5-yl)pyrimidine-5-carbonitrile

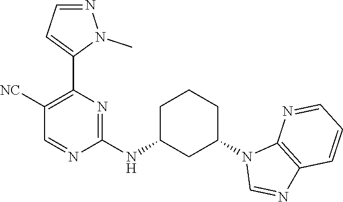 [SMILES: Cn1nccc1-c1nc(N[C@@H]2CCC[C@H](n3cnc4cccnc43)C2)ncc1C#N]

This compound was prepared according to the procedures described in Example 17, with 1-methyl-5-(4,4,5,5-tetramethyl-1,3,2-dioxaborolan-2-yl)-1H-pyrazole replacing 1,5-dimethyl-4-(4,4,5,5-tetramethyl-1,3,2-dioxaborolan-2-yl)-1H-pyrazole in Step 5. LCMS calculated for C₂₁H₂₂N₉(M+H)⁺: m/z=400.2; found: 400.2.

### Example 23. 2-(((1R,3S)-3-(3H-Imidazo[4,5-b]pyridin-3-yl)cyclohexyl)amino)-4-(1-methyl-5-(trifluoromethyl)-1H-pyrazol-4-yl)pyrimidine-5-carbonitrile

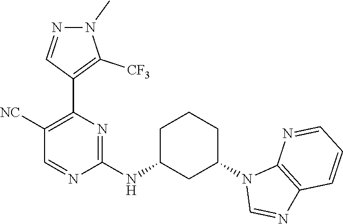 [SMILES: Cn1ncc(-c2nc(N[C@@H]3CCC[C@H](n4cnc5cccnc54)C3)ncc2C#N)c1C(F)(F)F]

This compound was prepared according to the procedures described in Example 17, with 1-methyl-4-(4,4,5,5-tetramethyl-1,3,2-dioxaborolan-2-yl)-5-(trifluoromethyl)-1H-pyrazole replacing 1,5-dimethyl-4-(4,4,5,5-tetramethyl-1,3,2-dioxaborolan-2-yl)-1H-pyrazole in Step 5. LCMS calculated for C₂₂H₂₁F₃N₉ (M+H)⁺: m/z=468.2; found: 468.1. ¹H NMR (500 MHz, DMSO-d₆, mixture of tautomers) δ 8.88-8.76 (m, 1.4H), 8.75 (s, 0.6H), 8.64-8.54 (m, 1H), 8.47-8.41 (m, 1H), 8.18-8.12 (m, 1H), 8.06 (s, 0.6H), 7.93 (s, 0.4H), 7.40-7.35 (m, 1H), 4.79-4.70 (m, 0.4H), 4.69-4.60 (m, 0.6H), 4.22-3.95 (m, 4H), 2.42-2.29 (m, 1H), 2.20-1.84 (m, 5H), 1.68-1.38 (m, 2H).

### Example 24. 2-(((1R,3S)-3-(3H-Imidazo[4,5-b]pyridin-3-yl)cyclohexyl)amino)-4-(1-ethyl-1H-pyrazol-4-yl)pyrimidine-5-carbonitrile

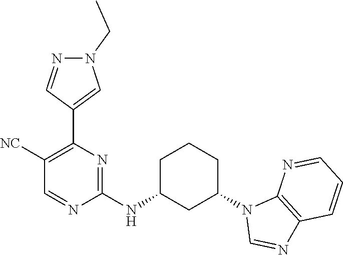 [SMILES: CCn1cc(-c2nc(N[C@@H]3CCC[C@H](n4cnc5cccnc54)C3)ncc2C#N)cn1]

This compound was prepared according to the procedures described in Example 17, with (1-ethyl-1H-pyrazol-4-yl)boronic acid replacing 1,5-dimethyl-4-(4,4,5,5-tetramethyl-1,3,2-dioxaborolan-2-yl)-1H-pyrazole in Step 5. LCMS calculated for C₂₂H₂₄N₉(M+H)⁺: m/z=414.2; found: 414.2.

### Example 25. 2-(((1R,3S)-3-(3H-Imidazo[4,5-b]pyridin-3-yl)cyclohexyl)amino)-4-(6,7-dihydro-5H-pyrazolo[5,1-b][1,3]oxazin-3-yl)pyrimidine-5-carbonitrile

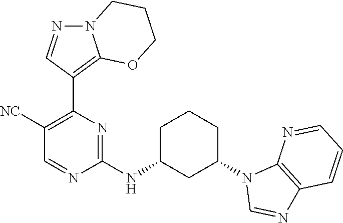 [SMILES: N#Cc1cnc(N[C@@H]2CCC[C@H](n3cnc4cccnc43)C2)nc1-c1cnn2c1OCCC2]

This compound was prepared according to the procedures described in Example 17, with 3-(4,4,5,5-tetramethyl-1,3,2-dioxaborolan-2-yl)-6,7-dihydro-5H-pyrazolo[5,1-b][1,3]oxazine replacing 1,5-dimethyl-4-(4,4,5,5-tetramethyl-1,3,2-dioxaborolan-2-yl)-1H-pyrazole in Step 5. LCMS calculated for C₂₃H₂₄N90 (M+H)⁺: m/z=442.2; found: 442.2.

### Example 26. 2-(((1R,3S)-3-(3H-Imidazo[4,5-b]pyridin-3-yl)cyclohexyl)amino)-4-(5-(hydroxymethyl)-1-methyl-1H-pyrazol-4-yl)pyrimidine-5-carbonitrile

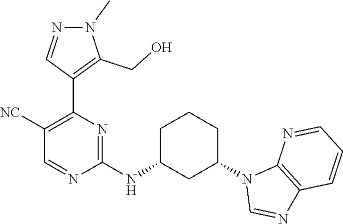 [SMILES: Cn1ncc(-c2nc(N[C@@H]3CCC[C@H](n4cnc5cccnc54)C3)ncc2C#N)c1CO]

### Step 1. 2-(((1R,3S)-3-(3H-Imidazo[4,5-b]pyridin-3-yl)cyclohexyl)amino)-4-(5-formyl-1-methyl-1H-pyrazol-4-yl)pyrimidine-5-carbonitrile

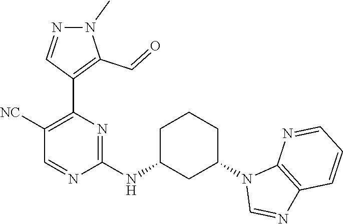 [SMILES: Cn1ncc(-c2nc(N[C@@H]3CCC[C@H](n4cnc5cccnc54)C3)ncc2C#N)c1C=O]

A vial containing 2-(((1R,3S)-3-(3H-imidazo[4,5-b]pyridin-3-yl)cyclohexyl)amino)-4-chloropyrimidine-5-carbonitrile (75 mg, 0.21 mmol), 1-methyl-4-(4,4,5,5-tetramethyl-1,3,2-dioxaborolan-2-yl)-1H-pyrazole-5-carbaldehyde (75 mg, 0.32 mmol), sodium carbonate (67 mg, 0.64 mmol), and Pd(dppf)Cl₂ CH₂Cl₂ (17 mg, 0.02 mmol) was evacuated and backfilled with nitrogen three times, followed by the addition of MeCN (2 mL) and water (400 μL). The vial was sealed and heated to 140° C. for 3 min. After cooling to rt, the mixture was diluted with MeCN, filtered through Celite and the filtrate was concentrated in vacuo. The obtained crude product was used in the next step without further purification. LCMS calculated for C₂₂H₂₂N₉O (M+H)⁺: m/z=428.2; found: 428.2.

### Step 2. 2-(((1R,3S)-3-(3H-Imidazo[4,5-b]pyridin-3-yl)cyclohexyl)amino)-4-(5-(hydroxymethyl)-1-methyl-1H-pyrazol-4-yl)pyrimidine-5-carbonitrile

To a vial containing 2-(((1R,3S)-3-(3H-imidazo[4,5-b]pyridin-3-yl)cyclohexyl)amino)-4-(5-formyl-1-methyl-1H-pyrazol-4-yl)pyrimidine-5-carbonitrile (15 mg, 0.03 mmol) as a solution in MeOH (1 mL) was added sodium borohydride (1.3 mg, 0.03 mmol) and left to stir at rt for 1 h. Upon completion, the mixture was diluted with MeCN and TFA and purified with prep-LCMS (XBridge C₁₈ column, eluting with a gradient of acetonitrile/water containing 0.1% TFA, at flow rate of 60 mL/min). LCMS calculated for C₂₂H₂₄N₉O (M+H)⁺: m/z=430.2; found: 430.2. ¹H NMR (500 MHz, DMSO-d₆, mixture of tautomers) δ 8.86 (d, J=6.2 Hz, 1H), 8.72 (s, 0.4H), 8.63 (s, 0.6H), 8.49-8.43 (m, 1H), 8.39 (d, J=7.8 Hz, 0.6H), 8.33 (d, J=7.9 Hz, 0.4H), 8.16 (dd, J=8.1, 1.5 Hz, 1H), 8.10 (s, 0.6H), 8.02 (s, 0.4H), 7.39 (dd, J=8.1, 4.7 Hz, 1H), 5.04-4.83 (m, 2H), 4.79-4.70 (m, 1H), 4.19-4.07 (m, 1H), 3.96 (s, 1.8H), 3.92 (s, 1.2H), 2.47-2.33 (m, 1H), 2.22-1.90 (m, 5H), 1.67-1.55 (m, 1H), 1.54-1.40 (m, 1H).

### Example 27. 2-(((1R,3S)-3-(3H-Imidazo[4,5-b]pyridin-3-yl)cyclohexyl)amino)-4-(1-isopropyl-1H-imidazol-4-yl)pyrimidine-5-carbonitrile

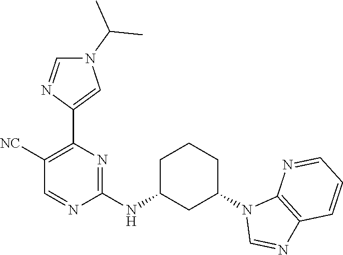 [SMILES: CC(C)n1cnc(-c2nc(N[C@@H]3CCC[C@H](n4cnc5cccnc54)C3)ncc2C#N)c1]

### Step 1. 2-(((1R,3S)-3-(3H-Imidazo[4,5-b]pyridin-3-yl)cyclohexyl)amino)-4-(1H-imidazol-4-yl)pyrimidine-5-carbonitrile

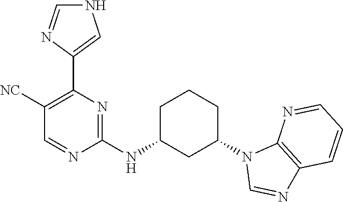 [SMILES: N#Cc1cnc(N[C@@H]2CCC[C@H](n3cnc4cccnc43)C2)nc1-c1c[nH]cn1]

A vial containing 2-(((1R,3S)-3-(3H-imidazo[4,5-b]pyridin-3-yl)cyclohexyl)amino)-4-chloropyrimidine-5-carbonitrile (162 mg, 0.46 mmol), NN-dimethyl-4-(4,4,5,5-tetramethyl-1,3,2-dioxaborolan-2-yl)-1H-imidazole-1-sulfonamide (179 mg, 0.59 mmol), sodium carbonate (146 mg, 1.37 mmol), and Pd(dppf)Cl₂ CH₂Cl₂ (37 mg, 0.05 mmol) was evacuated and backfilled with nitrogen three times, followed by the addition of MeCN (4 mL) and water (800 μL). The vial was sealed and heated to 80° C. for 1 h. After cooling to rt, the mixture was diluted with CH₂Cl₂, filtered through Celite and the filtrate was concentrated in vacuo. The resulting residue was purified by Teledyne ISCO CombiFlash to give the desired product as a tan foam (177 mg, 78% yield). The purified product was then treated with 4N HCl/dioxane (5 mL) and left to stir at rt for 1 h. Upon completion, the volatiles were removed in vacuo and the obtained crude product was used in the next step without further purification. LCMS calculated for C₂₀H₂₀N₉(M+H)⁺: m/z=386.2; found: 386.2.

### Step 2. 2-(((1R,3S)-3-(3H-Imidazo[4,5-b]pyridin-3-yl)cyclohexyl)amino)-4-(1-isopropyl-1H-imidazol-4-yl)pyrimidine-5-carbonitrile

To a vial containing 2-(((1R,3S)-3-(3H-imidazo[4,5-b]pyridin-3-yl)cyclohexyl)amino)-4-(1H-imidazol-4-yl)pyrimidine-5-carbonitrile (20 mg, 0.05 mmol) and cesium carbonate (34 mg, 0.1 mmol) as a suspension in MeCN (1 mL) and DIPEA (27 μL, 0.16 mmol) was added 2-iodopropane (6.2 μL, 0.06 mmol). The reaction vial was sealed and heated to 80° C. for 1 h. Upon completion, the mixture was diluted with MeCN and TFA and purified with prep-LCMS (XBridge C₁₈ column, eluting with a gradient of acetonitrile/water containing 0.1% TFA, at flow rate of 60 mL/min). LCMS calculated for C₂₃H₂₆N₉(M+H)⁺: m/z=428.2; found: 428.2.

### Example 28. 3-((1S,3R)-3-((5-Cyano-4-(1,5-dimethyl-1H-1,2,3-triazol-4-yl)pyrimidin-2-yl)amino)cyclohexyl)-3H-imidazo[4,5-b]pyridine-6-carbonitrile

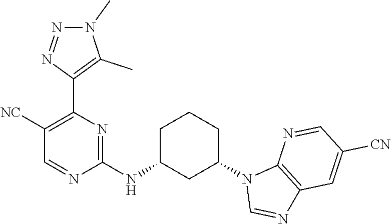 [SMILES: Cc1c(-c2nc(N[C@@H]3CCC[C@H](n4cnc5cc(C#N)cnc54)C3)ncc2C#N)nnn1C]

### Step 1. 3-((1S,3R)-3-((4-Chloro-5-cyanopyrimidin-2-yl)amino)cyclohexyl)-3H-imidazo[4,5-b]pyridine-6-carbonitrile

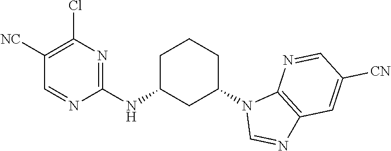 [SMILES: N#Cc1cnc2c(c1)ncn2[C@H]1CCC[C@@H](Nc2ncc(C#N)c(Cl)n2)C1]

To a solution of 2,4-dichloropyrimidine-5-carbonitrile (664 mg, 3.82 mmol) in t-butanol (4 mL) and 1,2-dichloroethane (4 mL) was added zinc(II) chloride (1.0 M in diethyl ether, 5.2 mL, 5.2 mmol). The solution was purged with nitrogen for 5 min, sealed and heated to 60° C. for 1 h, then cooled to rt. To this mixture was added a solution of 3-((1S,3R)-3-aminocyclohexyl)-3H-imidazo[4,5-b]pyridine-6-carbonitrile (837 mg, 3.47 mmol, Example 1 Step 4) in t-butanol (4 mL), 1,2-dichloroethane (4 mL) and DIPEA (1.5 mL, 8.67 mmol). The reaction mixture was then reheated to 60° C. and stirred for 3 h. Upon completion, the solution was cooled to rt and the volatiles were removed in vacuo. The resulting residue was purified by Teledyne ISCO CombiFlash (CH₂Cl₂/MeOH, up to 5% MeOH) to give the desired product. LCMS calculated for C₁₈H₁₆ClN₈ (M+H)⁺: m/z=379.1/381.1; found: 379.1/381.1.

### Step 2. 3-((1S,3R)-3-((5-Cyano-4-(1,5-dimethyl-1H-1,2,3-triazol-4-yl)pyrimidin-2-yl)amino)cyclohexyl)-3H-imidazo[4,5-b]pyridine-6-carbonitrile

A vial containing 3-((1S,3R)-3-((4-chloro-5-cyanopyrimidin-2-yl)amino)cyclohexyl)-3H-imidazo[4,5-b]pyridine-6-carbonitrile (10 mg, 0.03 mmol), (1,5-dimethyl-1H-1,2,3-triazol-4-yl)boronic acid (6 mg, 0.04 mmol), sodium carbonate (9 mg, 0.08 mmol), and Pd(dppf)Cl₂ CH₂Cl₂ (2 mg, 2.8 μmol) was evacuated and backfilled with nitrogen three times, followed by the addition of MeCN (1 mL) and water (200 μL). The vial was sealed and heated to 140° C. for 3 min. After cooling to rt, the mixture was filtered through a Silicycle SiliaPrep™ Thiol cartridge (Cat. #SPE-R51030B), washing with MeCN, and purified with prep-LCMS (XBridge C₁₈ column, eluting with a gradient of acetonitrile/water containing 0.1% TFA, at flow rate of 60 mL/min). LCMS calculated for C₂₂H₂₂N₁₁ (M+H)⁺: m/z=440.2; found: 440.2.

### Example 28A. Alternative Synthesis of 3-((1S,3R)-3-((5-Cyano-4-(1,5-dimethyl-1H-1,2,3-triazol-4-yl)pyrimidin-2-yl)amino)cyclohexyl)-3H-imidazo[4,5-b]pyridine-6-carbonitrile

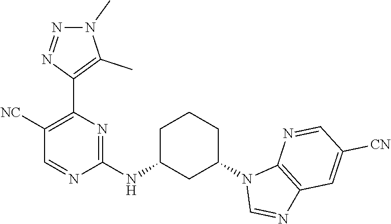 [SMILES: Cc1c(-c2nc(N[C@@H]3CCC[C@H](n4cnc5cc(C#N)cnc54)C3)ncc2C#N)nnn1C]

A vial containing 3-((1S,3R)-3-((4-chloro-5-cyanopyrimidin-2-yl)amino)cyclohexyl)-3H-imidazo[4,5-b]pyridine-6-carbonitrile (10 mg, 0.03 mmol; Example 9A, step 5), (1,5-dimethyl-1H-1,2,3-triazol-4-yl)boronic acid (6 mg, 0.04 mmol), sodium carbonate (9 mg, 0.08 mmol), and Pd(dppf)Cl₂ CH₂Cl₂ (2 mg, 2.8 μmol) was evacuated and backfilled with nitrogen three times, followed by the addition of MeCN (1 mL) and water (200 μL). The vial was sealed and heated to 140° C. for 3 min. After cooling to rt, the mixture was filtered through a Silicycle SiliaPrep™ Thiol cartridge (Cat. #SPE-R51030B), washing with MeCN, and purified with prep-LCMS (XBridge C₁₈ column, eluting with a gradient of acetonitrile/water containing 0.1% TFA, at flow rate of 60 mL/min). LCMS calculated for C₂₂H₂₂N₁₁ (M+H)⁺: m/z=440.2; found: 440.2.

### Example 29. 3-((1S,3R)-3-((5-Cyano-4-(1,5-dimethyl-1H-imidazol-2-yl)pyrimidin-2-yl)amino)cyclohexyl)-3H-imidazo[4,5-b]pyridine-6-carbonitrile

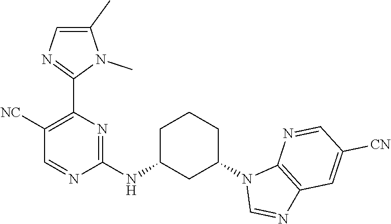 [SMILES: Cc1cnc(-c2nc(N[C@@H]3CCC[C@H](n4cnc5cc(C#N)cnc54)C3)ncc2C#N)n1C]

### Step 1. 3-((1S,3R)-3-((5-Cyano-4-(trimethylstannyl)pyrimidin-2-yl)amino)cyclohexyl)-3H-imidazo[4,5-b]pyridine-6-carbonitrile

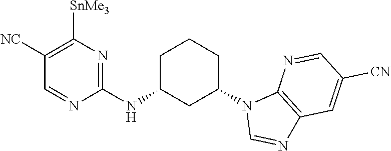

A vial containing 3-((1S,3R)-3-((4-chloro-5-cyanopyrimidin-2-yl)amino)cyclohexyl)-3H-imidazo[4,5-b]pyridine-6-carbonitrile (125 mg, 0.33 mmol, Example 9A, Step 5), tetrakis(triphenylphosphine)palladium(0) (38 mg, 0.03 mmol) and hexamethylditin (123 μL, 0.59 mmol) was evacuated and backfilled with nitrogen three times, followed by the addition of 1,4-dioxane (3 mL). The reaction was sealed and heated to 100° C. for 1 h. Upon cooling to rt, the volatiles were removed in vacuo. The resulting residue was purified by Teledyne ISCO CombiFlash to give the desired product as a yellow foam (84 mg, 50% yield). LCMS calculated for C₂₁H₂₅N₈Sn (M+H)⁺: m/z=509.1; found: 509.0.

### Step 2. 3-((1S,3R)-3-((5-Cyano-4-(1,5-dimethyl-1H-imidazol-2-yl)pyrimidin-2-yl)amino)cyclohexyl)-3H-imidazo[4,5-b]pyridine-6-carbonitrile

A vial containing 3-((1S,3R)-3-((5-cyano-4-(trimethylstannyl)pyrimidin-2-yl)amino)cyclohexyl)-3H-imidazo[4,5-b]pyridine-6-carbonitrile (15 mg, 0.03 mmol), 2-bromo-1,5-dimethyl-1H-imidazole (7 mg, 0.04 mmol), tetrakis(triphenylphosphine)palladium(0) (7 mg, 6 μmol) and cuprous iodide (2 mg, 0.59 μmol) was evacuated and backfilled with nitrogen three times, followed by the addition of 1,4-dioxane (1 mL). The reaction was sealed and heated to 100° C. for 1 h. After cooling to rt, the mixture was filtered through Celite and washed with CH₂Cl₂, followed by concentration of the filtrate in vacuo. The residue was then dissolved with CH₃CN and purified with prep-LCMS (XBridge C₁₈ column, eluting with a gradient of acetonitrile/water containing 0.1% TFA, at flow rate of 60 mL/min). LCMS calculated for C₂₃H₂₃N₁₀ (M+H)⁺: m/z=439.2; found: 439.2.

### Example 30. 3-((1S,3R)-3-((5-Cyano-4-((1,5-dimethyl-1H-pyrazol-4-yl)amino)pyrimidin-2-yl)amino)cyclohexyl)-3H-imidazo[4,5-b]pyridine-6-carbonitrile

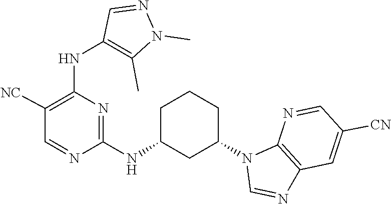 [SMILES: Cc1c(Nc2nc(N[C@@H]3CCC[C@H](n4cnc5cc(C#N)cnc54)C3)ncc2C#N)cnn1C]

A vial containing a solution of 3-((1S,3R)-3-((4-chloro-5-cyanopyrimidin-2-yl)amino)cyclohexyl)-3H-imidazo[4,5-b]pyridine-6-carbonitrile (10 mg, 0.03 mmol, Example 28, Step 1) and 1,5-dimethyl-1H-pyrazol-4-amine (4 mg, 0.04 mmol) in 1-butanol (1 mL) was sealed and heated to 100° C. for 1 h. After cooling to rt, the mixture was then diluted with CH₃CN and TFA and purified with prep-LCMS (XBridge C₁₈ column, eluting with a gradient of acetonitrile/water containing 0.1% TFA, at flow rate of 60 mL/min). LCMS calculated for C₂₃H₂₄N₁₁ (M+H)⁺: m/z=454.2; found: 454.2.

### Example 31. 1-((1S,3R)-3-((5-Cyano-4-(1-(2,2-difluoroethyl)-1H-pyrazol-4-yl)pyrimidin-2-yl)amino)cyclohexyl)-1H-imidazo[4,5-c]pyridine-7-carbonitrile

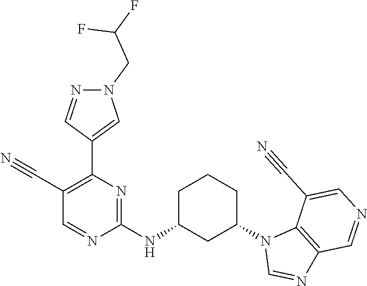 [SMILES: N#Cc1cnc(N[C@@H]2CCC[C@H](n3cnc4cncc(C#N)c43)C2)nc1-c1cnn(CC(F)F)c1]

### Step 1. tert-Butyl ((1R,3S)-3-((3-bromo-5-nitropyridin-4-yl)amino)cyclohexyl)carbamate

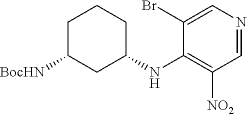 [SMILES: CC(C)(C)OC(=O)N[C@@H]1CCC[C@H](Nc2c(Br)cncc2[N+](=O)[O-])C1]

A mixture of tert-butyl ((1R,3S)-3-aminocyclohexyl)carbamate (100 mg, 0.47 mmol), 3-bromo-4-chloro-5-nitropyridine (122 mg, 0.51 mmol), and TEA (195 μL, 1.4 mmol) in DMF (1.2 mL) was stirred at 60° C. for 1 h. Upon completion, the reaction mixture was cooled to rt and poured into ice water, and the resulting solid was collected by filtration. The solid was washed with water, hexanes, and left to dry under vacuum. The crude solid was taken on to the next step without further purification. LCMS calculated for C₁₆H₂₄BrN₄O₄(M+H)⁺: m/z=415.1/417.1; found: 415.1/417.1.

### Step 2. tert-Butyl ((1R,3S)-3-((3-amino-5-bromopyridin-4-yl)amino)cyclohexyl)carbamate

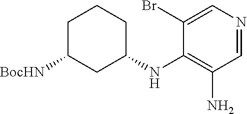 [SMILES: CC(C)(C)OC(=O)N[C@@H]1CCC[C@H](Nc2c(N)cncc2Br)C1]

To a vial containing tert-butyl ((1R,3S)-3-((3-bromo-5-nitropyridin-4-yl)amino)cyclohexyl)carbamate (194 mg, 0.47 mmol) as a solution in THE (2 mL), MeOH (2 mL), and water (2 mL) was added iron powder (130 mg, 2.3 mmol) and ammonium chloride (150 mg, 2.8 mmol). The vial was sealed and heated to 60° C. for 1 h. After cooling to rt, the reaction mixture was diluted with CH₂Cl₂(5 mL) and filtered over Celite. The filtrate was concentrated in vacuo and the resulting crude residue was used in the next step with no further purification. LCMS calculated for C₁₆H₂₆BrN₄O₂(M+H)⁺: m/z=385.1/387.1; found: 385.2/387.2.

### Step 3. tert-Butyl ((1R,3S)-3-(7-bromo-1H-imidazo[4,5-c]pyridin-1-yl)cyclohexyl)carbamate

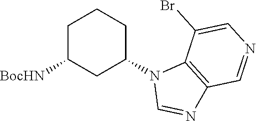 [SMILES: CC(C)(C)OC(=O)N[C@@H]1CCC[C@H](n2cnc3cncc(Br)c32)C1]

A vial containing tert-butyl ((1R,3S)-3-((3-amino-5-bromopyridin-4-yl)amino)cyclohexyl)carbamate (90 mg, 0.23 mmol) and trimethyl orthoformate (153 μL, 1.4 mmol) as a solution in toluene (1 mL) was sealed and heated to 120° C. for 3 h. Upon completion, the reaction mixture was concentrated in vacuo. The resulting residue was purified by Biotage Isolera (CH₂Cl₂/MeOH, up to 10% MeOH) to give the desired product. LCMS calculated for C₁₇H₂₄BrN₄O₂(M+H)⁺: m/z=395.1/397.1; found: 395.2/397.2.

### Step 4. 1-((1S,3R)-3-Aminocyclohexyl)-1H-imidazo[4,5-c]pyridine-7-carbonitrile

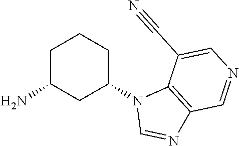 [SMILES: N#Cc1cncc2ncn([C@H]3CCC[C@@H](N)C3)c12]

A microwave vial containing tert-butyl ((1R,3S)-3-(7-bromo-1H-imidazo[4,5-c]pyridin-1-yl)cyclohexyl)carbamate (500 mg, 1.26 mmol), Zn(CN)₂ (297 mg, 2.53 mmol), zinc powder (331 mg, 5.1 mmol), and Pd(dppf)Cl₂ CH₂Cl₂(155 mg, 0.19 mmol) was evacuated and backfilled with nitrogen three times, followed by the addition of DMF (5 mL). The vessel was irradiated at 120° C. for 1 h using a Biotage Initator+ Microwave Synthesizer. After cooling to rt, the reaction mixture was filtered over Celite and washed with CH₂Cl₂, followed by concentration of the filtrate in vacuo to remove CH₂Cl₂. The remaining DMF solution was diluted with MeOH (2 mL) and poured into ice water, and the resulting solid was collected by filtration. The solid was washed with water, hexanes, and left to dry under vacuum. The residue was then purified by Biotage Isolera (CH₂Cl₂/MeOH, up to 15% MeOH). The purified material was then dissolved in MeOH (3 mL) and 4N HCl/dioxane (5 mL) and left to stir at rt for 1 h. Upon completion, the reaction mixture was concentrated in vacuo. The obtained crude product was used in the next step without further purification. LCMS calculated for C₁₃H₁₆N₅(M+H)⁺: m/z=242.2; found: 242.2.

### Step 5. 1-((1S,3R)-3-((4-Chloro-5-cyanopyrimidin-2-yl)amino)cyclohexyl)-1H-imidazo[4,5-c]pyridine-7-carbonitrile

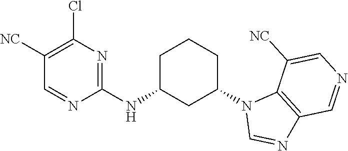 [SMILES: N#Cc1cnc(N[C@@H]2CCC[C@H](n3cnc4cncc(C#N)c43)C2)nc1Cl]

To a solution of 2,4-dichloropyrimidine-5-carbonitrile (12 mg, 0.07 mmol) in tert-BuOH (1 mL) and 1,2-dichloroethane (1 mL) was added zinc(II) chloride (0.5 M in diethyl ether, 193 μL, 0.1 mmol). The solution was purged with nitrogen for 5 min, sealed and heated to 60° C. for 1 h, then cooled to rt. To this vial was added 1-((1S,3R)-3-aminocyclohexyl)-1H-imidazo[4,5-c]pyridine-7-carbonitrile (15 mg, 0.06 mmol) and DIPEA (45 μL, 0.26 mmol). The reaction mixture was then reheated to 60° C. and stirred for 2 h. Upon completion, the solution was cooled rt and the volatiles were removed in vacuo. The resulting residue was purified by Teledyne ISCO CombiFlash (CH₂Cl₂/MeOH, up to 5% MeOH) to give the desired product. LCMS calculated for C₁₈H₁₆ClN₈ (M+H)⁺: m/z=379.1/381.1; found: 379.1/381.1.

### Step 6. 1-((1S,3R)-3-((5-Cyano-4-(1-(2,2-difluoroethyl)-1H-pyrazol-4-yl)pyrimidin-2-yl)amino)cyclohexyl)-1H-imidazo[4,5-c]pyridine-7-carbonitrile

A mixture of 1-((1S,3R)-3-((4-chloro-5-cyanopyrimidin-2-yl)amino)cyclohexyl)-1H-imidazo[4,5-c]pyridine-7-carbonitrile (15 mg, 0.04 mmol), 1-(2,2-difluoroethyl)-4-(4,4,5,5-tetramethyl-1,3,2-dioxaborolan-2-yl)-1H-pyrazole (11 mg, 0.04 mmol), and sodium carbonate (13 mg, 0.16 mmol) was dissolved in MeCN (1 mL) and water (200 μL). The solution was purged with nitrogen for 2 min, followed by addition of Pd(dppf)Cl₂ CH₂Cl₂ (3.2 mg, 4 mol). The vial was sealed and the reaction was stirred at 140° C. for 5 min before being cooled rt. The solution was diluted with MeOH, filtered through a Silicycle SiliaPrep™ Thiol cartridge (Cat. #SPE-R51030B), and purified with prep-LCMS (XBridge C₁₈ column, eluting with a gradient of acetonitrile/water containing 0.1% TFA, at flow rate of 60 mL/min). LCMS calculated for C₂₃H₂₁F₂N₁₀ (M+H)⁺: m/z=475.2; found: 475.3.

### Example 32. 1-((1S,3R)-3-((5-Cyano-4-(1-(2-hydroxyethyl)-1H-pyrazol-4-yl)pyrimidin-2-yl)amino)cyclohexyl)-1H-imidazo[4,5-c]pyridine-7-carbonitrile

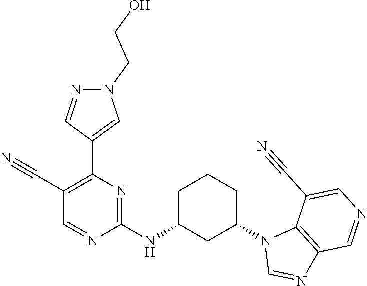 [SMILES: N#Cc1cnc(N[C@@H]2CCC[C@H](n3cnc4cncc(C#N)c43)C2)nc1-c1cnn(CCO)c1]

This compound was prepared according to the procedures described in Example 31, with 2-(4-(4,4,5,5-tetramethyl-1,3,2-dioxaborolan-2-yl)-1H-pyrazol-1-yl)ethan-1-ol replacing 1-(2,2-difluoroethyl)-4-(4,4,5,5-tetramethyl-1,3,2-dioxaborolan-2-yl)-1H-pyrazole in Step 6. LCMS calculated for C₂₃H₂₃N₁₀O (M+H)⁺: m/z=455.2; found: 455.2. ¹H NMR (600 MHz, DMSO-d₆, mixture of tautomers) δ 9.27 (s, 1H), 8.88 (d, J=3.6 Hz, 1H), 8.85 (s, 0.6H), 8.81 (s, 0.4H), 8.71 (s, 0.4H), 8.62 (s, 0.6H), 8.54 (s, 0.6H), 8.45 (s, 0.4H), 8.36 (dd, J=19.8, 7.7 Hz, 1H), 8.29 (s, 0.6H), 8.17 (s, 0.4H), 4.96-4.80 (m, 1H), 4.29-4.24 (m, 2H), 4.21-4.04 (m, 1H), 3.77 (dt, J=10.2, 5.3 Hz, 2H), 2.49-2.44 (m, 1H), 2.26-2.16 (m, 1H), 2.09-1.95 (m, 3H), 1.94-1.86 (m, 1H), 1.68-1.40 (m, 2H).

### Example 33. 1-((1S,3R)-3-((5-Cyano-4-(1-isopropyl-1H-pyrazol-4-yl)pyrimidin-2-yl)amino)cyclohexyl)-1H-imidazo[4,5-c]pyridine-7-carbonitrile

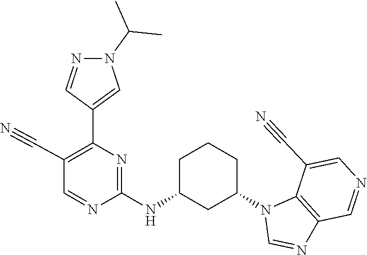 [SMILES: CC(C)n1cc(-c2nc(N[C@@H]3CCC[C@H](n4cnc5cncc(C#N)c54)C3)ncc2C#N)cn1]

This compound was prepared according to the procedures described in Example 31, with 1-isopropyl-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₄H₂₅N₁₀ (M+H)⁺: m/z=453.2; found: 453.2.

### Example 34. 1-((1S,3R)-3-((5-Cyano-4-(1-cyclopropyl-1H-pyrazol-4-yl)pyrimidin-2-yl)amino)cyclohexyl)-1H-imidazo[4,5-c]pyridine-7-carbonitrile

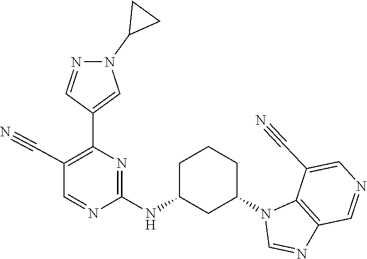 [SMILES: N#Cc1cnc(N[C@@H]2CCC[C@H](n3cnc4cncc(C#N)c43)C2)nc1-c1cnn(C2CC2)c1]

This compound was prepared according to the procedures described in Example 31, with 1-cyclopropyl-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₄H₂₃N₁₀ (M+H)⁺: m/z=451.2; found: 451.2.

### Example 35. 3-((1R,3S)-3-((5-Cyano-4-(1-(2,2-difluoroethyl)-1H-pyrazol-4-yl)pyrimidin-2-yl)amino)cyclohexyl)-3H-imidazo[4,5-b]pyridine-6-carbonitrile

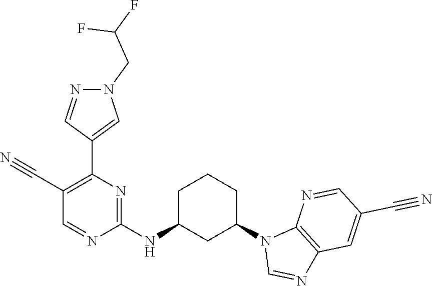 [SMILES: N#Cc1cnc2c(c1)ncn2[C@@H]1CCC[C@H](Nc2ncc(C#N)c(-c3cnn(CC(F)F)c3)n2)C1]

### Step 1. 3-((1R,3S)-3-Aminocyclohexyl)-3H-imidazo[4,5-b]pyridine-6-carbonitrile

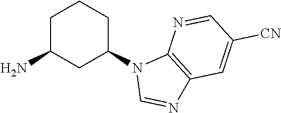 [SMILES: N#Cc1cnc2c(c1)ncn2[C@@H]1CCC[C@H](N)C1]

This compound was prepared according to the procedures described in Example 9A, steps 1-4, with tert-butyl ((1S,3R)-3-aminocyclohexyl)carbamate replacing tert-butyl ((1R,3S)-3-aminocyclohexyl)carbamate in Step 1. LCMS calculated for C₁₃H₁₆N₅(M+H)⁺: m/z=242.1; found: 242.2.

### Step 2. 3-((1R,3S)-3-((5-Cyano-4-(1-(2,2-difluoroethyl)-1H-pyrazol-4-yl)pyrimidin-2-yl)amino)cyclohexyl)-3H-imidazo[4,5-b]pyridine-6-carbonitrile

To a solution of 3-((1R,3S)-3-aminocyclohexyl)-3H-imidazo[4,5-b]pyridine-6-carbonitrile (12 mg, 0.05 mmol) in EtOH (1 mL) was added 4-(1-(2,2-difluoroethyl)-1H-pyrazol-4-yl)-2-(methylsulfonyl)pyrimidine-5-carbonitrile (Intermediate 1, 15 mg, 0.05 mmol) and DIPEA (50 μL, 0.29 mmol). The reaction vial was sealed and heated to 60° C. for 15 min. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₁F₂N₁₀ (M+H)⁺: m/z=475.2; found: 475.2.

### Example 36. 2-(((1R,3S)-3-(7-Chloro-5-fluoro-1H-benzo[d]imidazol-1-yl)cyclohexyl)amino)-4-(1-(2,2-difluoroethyl)-1H-pyrazol-4-yl)pyrimidine-5-carbonitrile

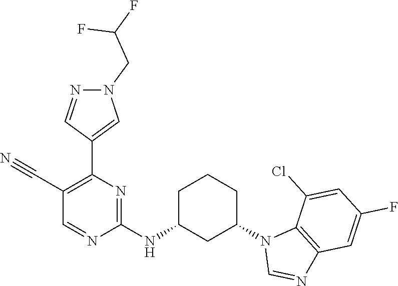 [SMILES: N#Cc1cnc(N[C@@H]2CCC[C@H](n3cnc4cc(F)cc(Cl)c43)C2)nc1-c1cnn(CC(F)F)c1]

### Step 1. tert-Butyl ((1R,3S)-3-((2-chloro-4-fluoro-6-nitrophenyl)amino)cyclohexyl)carbamate

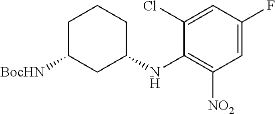 [SMILES: CC(C)(C)OC(=O)N[C@@H]1CCC[C@H](Nc2c(Cl)cc(F)cc2[N+](=O)[O-])C1]

A mixture of tert-butyl ((1R,3S)-3-aminocyclohexyl)carbamate (100 mg, 0.47 mmol), 1-chloro-2,5-difluoro-3-nitrobenzene (90 mg, 0.47 mmol), and DIPEA (122 μL, 0.7 mmol) in DMSO (1 mL) was heated to 60° C. and stirred for 1 h. Upon completion, the reaction mixture was cooled to rt and poured into ice water, and the resulting solid was collected by filtration. The solid was washed with water, hexanes, and left to dry under vacuum. The crude residue was taken on to the next step without further purification. LCMS calculated for C₁₃H₁₆C₁FN₃O₄(M+H−C₄H₈): m/z=332.1/334.1; found: 332.0/334.0.

### Step 2. tert-Butyl ((1R,3S)-3-((2-amino-6-chloro-4-fluorophenyl)amino)cyclohexyl)carbamate

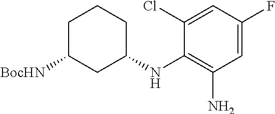 [SMILES: CC(C)(C)OC(=O)N[C@@H]1CCC[C@H](Nc2c(N)cc(F)cc2Cl)C1]

To a vial containing tert-butyl ((1R,3S)-3-((2-chloro-4-fluoro-6-nitrophenyl)amino)cyclohexyl)carbamate (181 mg, 0.47 mmol) as a solution in THE (1 mL), MeOH (1 mL), and water (1 mL) was added iron powder (130 mg, 2.3 mmol) and ammonium chloride (150 mg, 2.8 mmol). The vial was sealed and heated to 60° C. for 1 h. After cooling to rt, the reaction mixture was diluted with CH₂Cl₂(5 mL) and filtered over Celite. The filtrate was concentrated in vacuo and the resulting crude residue was used in the next step with no further purification. LCMS calculated for C₁₇H₂₆C₁FN₃O₂(M+H)⁺: m/z=358.2; found: 358.2.

### Step 3. tert-Butyl ((1R,3S)-3-(7-chloro-5-fluoro-1H-benzo[d]imidazol-1-yl)cyclohexyl)carbamate

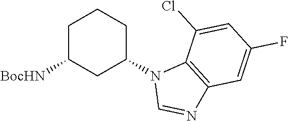 [SMILES: CC(C)(C)OC(=O)N[C@@H]1CCC[C@H](n2cnc3cc(F)cc(Cl)c32)C1]

A vial containing tert-butyl ((1R,3S)-3-((2-amino-6-chloro-4-fluorophenyl)amino)cyclohexyl)carbamate (167 mg, 0.47 mmol) and trimethyl orthoformate (310 μL, 2.8 mmol) as a solution in toluene (3 mL) was sealed and heated to 120° C. for 16 h. Upon completion, the reaction mixture was concentrated in vacuo. The resulting residue was purified by Biotage Isolera (CH₂Cl₂/MeOH, up to 10% MeOH) to give the desired product (126 mg, 73% yield). LCMS calculated for C₁₈H₂₄C₁FN₃O₂(M+H)⁺: m/z=368.2/370.2; found: 368.1/370.1.

### Step 4. (1R,3S)-3-(7-Chloro-5-fluoro-1H-benzo[d]imidazol-1-yl)cyclohexan-1-amine

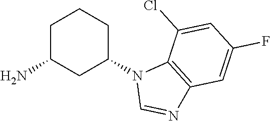 [SMILES: N[C@@H]1CCC[C@H](n2cnc3cc(F)cc(Cl)c32)C1]

To a solution of tert-butyl ((1R,3S)-3-(7-chloro-5-fluoro-1H-benzo[d]imidazol-1-yl)cyclohexyl)carbamate (126 mg, 0.34 mmol) in MeOH (1 mL) was added 4N HCl/dioxane (1 mL) and left to stir at rt for 1 h. Upon completion, the reaction mixture was concentrated in vacuo. The obtained crude product was used in the next step without further purification. LCMS calculated for C₁₃H₁₆C₁FN₃ (M+H)⁺: m/z=268.1/270.1; found: 268.2/270.2.

### Step 5. 2-(((1R,3S)-3-(7-Chloro-5-fluoro-1H-benzo[d]imidazol-1-yl)cyclohexyl)amino)-4-(1-(2,2-difluoroethyl)-1H-pyrazol-4-yl)pyrimidine-5-carbonitrile

To a solution of (1R,3S)-3-(7-chloro-5-fluoro-1H-benzo[d]imidazol-1-yl)cyclohexan-1-amine (12 mg, 0.045 mmol) in n-BuOH (1 mL) was added 4-(1-(2,2-difluoroethyl)-1H-pyrazol-4-yl)-2-(methylsulfonyl)pyrimidine-5-carbonitrile (Intermediate 1, 14 mg, 0.045 mmol) and DIPEA (47 μL, 0.27 mmol). The reaction vial was sealed and heated to 60° C. for 15 min. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₁ClF₃N₈(M+H)⁺: m/z=501.2; found: 501.2.

### Example 37. 3-((1S,3R)-3-((5-Cyano-4-(1-(1-hydroxy-2-methylpropan-2-yl)-1H-pyrazol-4-yl)pyrimidin-2-yl)amino)cyclohexyl)-3H-imidazo[4,5-b]pyridine-6-carbonitrile

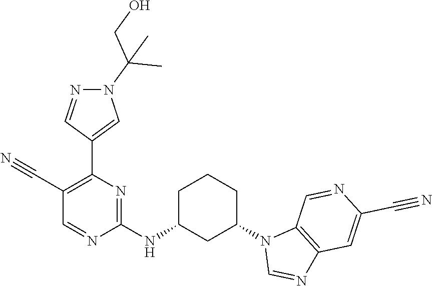 [SMILES: CC(C)(CO)n1cc(-c2nc(N[C@@H]3CCC[C@H](n4cnc5cc(C#N)ncc54)C3)ncc2C#N)cn1]

A mixture of 1-((1S,3R)-3-((4-chloro-5-cyanopyrimidin-2-yl)amino)cyclohexyl)-1H-benzo[d]imidazole-5-carbonitrile (35 mg, 0.09 mmol, Example 9A, Step 5), 2-methyl-2-(4-(4,4,5,5-tetramethyl-1,3,2-dioxaborolan-2-yl)-1H-pyrazol-1-yl)propan-1-ol (27 mg, 0.10 mmol), and sodium carbonate (29 mg, 0.28 mmol) was dissolved in MeCN (1 mL) and water (200 μL). The solution was purged with nitrogen for 2 min, followed by addition of Pd(dppf)Cl₂ CH₂Cl₂ (15 mg, 0.02 mmol). The vial was sealed and the reaction was stirred at 140° C. for 5 min before being cooled to rt, diluted with CH₂Cl₂ (5 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₅H₂₇N₁₀O (M+H)⁺: m/z=483.2; found: 483.2. ¹H NMR (500 MHz, DMSO-d₆, mixture of tautomers) δ 8.85-8.78 (m, 2H), 8.74-8.67 (m, 1.45H), 8.60 (s, 0.55H), 8.54 (s, 0.55H), 8.45 (s, 0.45H), 8.36 (s, 0.55H), 8.33-8.27 (m, 1H), 8.13 (s, 0.45H), 4.87-4.69 (m, 1H), 4.22-4.07 (m, 3H), 2.49-2.43 (m, 0.45H), 2.38-2.30 (m, 0.55H), 2.15-1.88 (m, 5H), 1.74-1.53 (m, 1H), 1.53-1.42 (m, 1H), 1.11 (s, 3H), 1.08 (s, 3H).

### Example 38. 3-((1S,3R)-3-((5-Cyano-4-(1-cyclobutyl-1H-pyrazol-4-yl)pyrimidin-2-yl)amino)cyclohexyl)-3H-imidazo[4,5-b]pyridine-6-carbonitrile

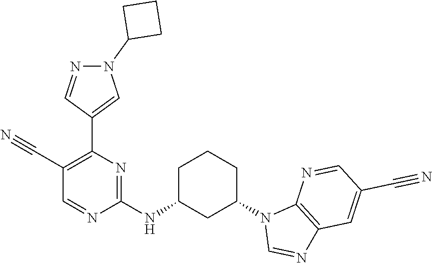 [SMILES: N#Cc1cnc2c(c1)ncn2[C@H]1CCC[C@@H](Nc2ncc(C#N)c(-c3cnn(C4CCC4)c3)n2)C1]

A mixture of 1-((1S,3R)-3-((4-chloro-5-cyanopyrimidin-2-yl)amino)cyclohexyl)-1H-benzo[d]imidazole-5-carbonitrile (50 mg, 0.13 mmol, Example 9A, Step 5), 1-cyclobutyl-4-(4,4,5,5-tetramethyl-1,3,2-dioxaborolan-2-yl)-1H-pyrazole (36 mg, 0.14 mmol), and sodium carbonate (42 mg, 0.40 mmol) was dissolved in MeCN (1 mL) and water (200 μL). The solution was purged with nitrogen for 2 min, followed by addition of Pd(dppf)Cl₂ CH₂Cl₂ (22 mg, 0.03 mmol). The vial was sealed and the reaction was stirred at 140° C. for 5 min before being cooled to rt, diluted with CH₂Cl₂ (5 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₅H₂₅N₁₀ (M+H)⁺: m/z=465.2; found: 465.2. ¹H NMR (500 MHz, DMSO-d₆, mixture of tautomers) δ 8.87-8.78 (m, 2H), 8.72-8.68 (m, 1.45H), 8.61 (s, 0.55H), 8.55 (s, 0.55H), 8.45 (s, 0.45H), 8.36 (s, 0.55H), 8.34-8.27 (m, 1H), 8.17 (s, 0.45H), 5.06-4.94 (m, 1H), 4.87-4.68 (m, 1H), 4.23-4.07 (m, 1H), 2.58-2.30 (m, 5H), 2.15-1.91 (m, 5H), 1.90-1.75 (m, 2H), 1.74-1.53 (m, 1H), 1.53-1.42 (m, 1H).

### Example 39. 2-(((1R,3S)-3-(7-Bromo-1H-imidazo[4,5-c]pyridin-1-yl)cyclohexyl)amino)-4-(1-(2,2-difluoroethyl)-1H-pyrazol-4-yl)pyrimidine-5-carbonitrile

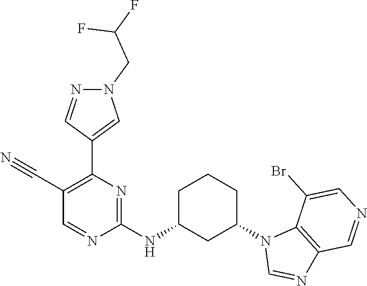 [SMILES: N#Cc1cnc(N[C@@H]2CCC[C@H](n3cnc4cncc(Br)c43)C2)nc1-c1cnn(CC(F)F)c1]

This compound was prepared according to the procedures described in Example 36, with 3-bromo-4-chloro-5-nitropyridine replacing 1-chloro-2,5-difluoro-3-nitrobenzene in Step 1. LCMS calculated for C₂₂H₂₁BrF₂N₉(M+H)⁺: m/z=528.1/530.1; found: 528.1/530.1.

### Example 40. 2-(((1R,3S)-3-(7-Bromo-5-fluoro-1H-benzo[d]imidazol-1-yl)cyclohexyl)amino)-4-(1-(2,2-difluoroethyl)-1H-pyrazol-4-yl)pyrimidine-5-carbonitrile

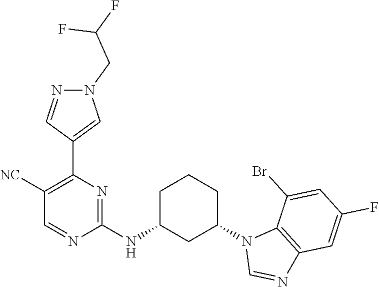 [SMILES: N#Cc1cnc(N[C@@H]2CCC[C@H](n3cnc4cc(F)cc(Br)c43)C2)nc1-c1cnn(CC(F)F)c1]

This compound was prepared according to the procedures described in Example 36, with 1-bromo-2,5-difluoro-3-nitrobenzene replacing 1-chloro-2,5-difluoro-3-nitrobenzene in Step 1. LCMS calculated for C₂₃H₂₁BrF₃N₈(M+H)⁺: m/z=545.1/547.1; found: 545.1/547.1.

### Example 41. 2-(((1R,3S)-3-(7-Bromo-1H-benzo[d]imidazol-1-yl)cyclohexyl)amino)-4-(1-(2,2-difluoroethyl)-1H-pyrazol-4-yl)pyrimidine-5-carbonitrile

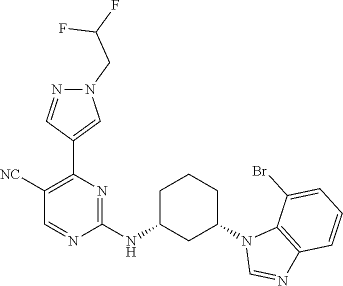 [SMILES: N#Cc1cnc(N[C@@H]2CCC[C@H](n3cnc4cccc(Br)c43)C2)nc1-c1cnn(CC(F)F)c1]

This compound was prepared according to the procedures described in Example 36, with 1-bromo-2-fluoro-3-nitrobenzene replacing 1-chloro-2,5-difluoro-3-nitrobenzene in Step 1. LCMS calculated for C₂₃H₂₂BrF₂N₈(M+H)⁺: m/z=527.1/529.1; found: 527.1/529.1.

### Example 42. 2-(((1R,3S)-3-(7-Bromo-6-chloro-1H-imidazo[4,5-c]pyridin-1-yl)cyclohexyl)amino)-4-(1-(2,2-difluoroethyl)-1H-pyrazol-4-yl)pyrimidine-5-carbonitrile

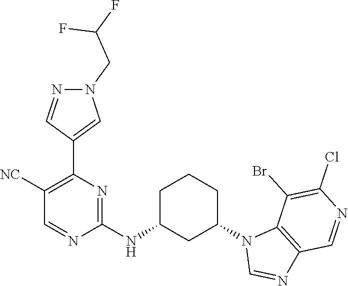 [SMILES: N#Cc1cnc(N[C@@H]2CCC[C@H](n3cnc4cnc(Cl)c(Br)c43)C2)nc1-c1cnn(CC(F)F)c1]

This compound was prepared according to the procedures described in Example 36, with 3-bromo-2,4-dichloro-5-nitropyridine replacing 1-chloro-2,5-difluoro-3-nitrobenzene in Step 1. LCMS calculated for C₂₂H₂₀BrClF₂N₉(M+H)⁺: m/z=562.1/564.1/566.1; found: 562.1/564.1/566.1.

### Example 43. 1-((1S,3R)-3-((5-Cyano-4-(1-(2,2-difluoroethyl)-1H-pyrazol-4-yl)pyrimidin-2-yl)amino)cyclohexyl)-1H-benzo[d]imidazole-6-carbonitrile

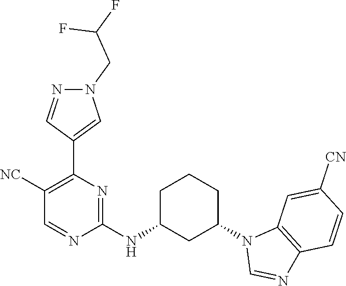 [SMILES: N#Cc1ccc2ncn([C@H]3CCC[C@@H](Nc4ncc(C#N)c(-c5cnn(CC(F)F)c5)n4)C3)c2c1]

This compound was prepared according to the procedures described in Example 36, with 3-fluoro-4-nitrobenzonitrile replacing 1-chloro-2,5-difluoro-3-nitrobenzene in Step 1. LCMS calculated for C₂₄H₂₂F₂N₉ (M+H)⁺: m/z=474.2; found: 474.3.

### Example 45. 2-(((1R,3S)-3-(7-Chloro-1H-imidazo[4,5-c]pyridin-1-yl)cyclohexyl)amino)-4-(1-(2,2-difluoroethyl)-1H-pyrazol-4-yl)pyrimidine-5-carbonitrile

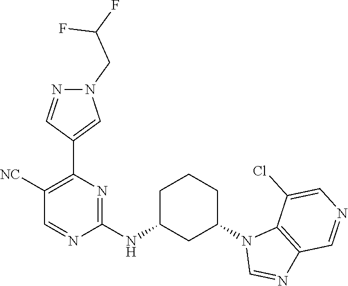 [SMILES: N#Cc1cnc(N[C@@H]2CCC[C@H](n3cnc4cncc(Cl)c43)C2)nc1-c1cnn(CC(F)F)c1]

This compound was prepared according to the procedures described in Example 36, with 3,4-dichloro-5-nitropyridine replacing 1-chloro-2,5-difluoro-3-nitrobenzene in Step 1. LCMS calculated for C₂₂H₂₁ClF₂N₉(M+H)⁺: m/z=484.2/486.2; found: 484.2/486.1.

### Example 46. 1-((1S,3R)-3-((5-Cyano-4-(1-(2,2-difluoroethyl)-1H-pyrazol-4-yl)pyrimidin-2-yl)amino)cyclohexyl)-1H-imidazo[4,5-c]pyridine-6-carbonitrile

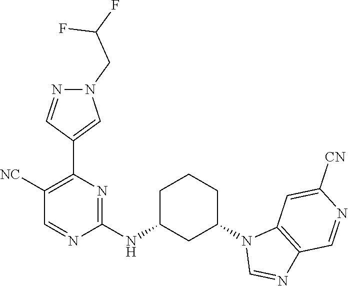 [SMILES: N#Cc1cc2c(cn1)ncn2[C@H]1CCC[C@@H](Nc2ncc(C#N)c(-c3cnn(CC(F)F)c3)n2)C1]

### Step 1. tert-Butyl ((1R,3S)-3-((2-bromo-5-nitropyridin-4-yl)amino)cyclohexyl)carbamate

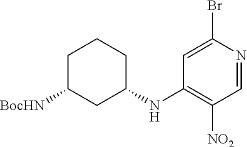 [SMILES: CC(C)(C)OC(=O)N[C@@H]1CCC[C@H](Nc2cc(Br)ncc2[N+](=O)[O-])C1]

A mixture of tert-butyl ((1R,3S)-3-aminocyclohexyl)carbamate (108 mg, 0.50 mmol), 2-bromo-4-chloro-5-nitropyridine (100 mg, 0.42 mmol), and DIPEA (110 μL, 0.63 mmol) in EtOH (3 mL) was heated to 80° C. and stirred for 1 h. Upon completion, the reaction mixture was cooled to rt and concentrated in vacuo. The crude residue was taken on to the next step without further purification. LCMS calculated for C₁₆H₂₄BrN₄O₄(M+H)⁺: m/z=415.1/417.1; found: 415.0/417.0.

### Step 2. tert-Butyl ((1R,3S)-3-((5-amino-2-bromopyridin-4-yl)amino)cyclohexyl)carbamate

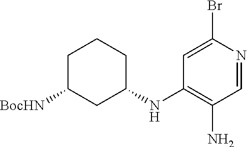 [SMILES: CC(C)(C)OC(=O)N[C@@H]1CCC[C@H](Nc2cc(Br)ncc2N)C1]

To a vial containing tert-butyl ((1R,3S)-3-((2-bromo-5-nitropyridin-4-yl)amino)cyclohexyl)carbamate (175 mg, 0.42 mmol) as a solution in THE (1 mL), MeOH (1 mL), and water (1 mL) was added iron powder (118 mg, 2.1 mmol) and ammonium chloride (135 mg, 2.5 mmol). The vial was sealed and heated to 60° C. for 1 h. After cooling to rt, the reaction mixture was diluted with CH₂Cl₂(5 mL) and filtered over Celite. The filtrate was concentrated in vacuo and the resulting crude residue was used in the next step with no further purification. LCMS calculated for C₁₆H₂₆BrN₄O₂(M+H)⁺: m/z=385.1/387.1; found: 385.1/387.1.

### Step 3. tert-Butyl ((1R,3S)-3-(6-bromo-1H-imidazo[4,5-c]pyridin-1-yl)cyclohexyl)carbamate

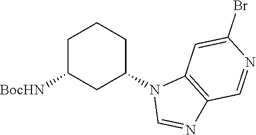 [SMILES: CC(C)(C)OC(=O)N[C@@H]1CCC[C@H](n2cnc3cnc(Br)cc32)C1]

A vial containing tert-butyl ((1R,3S)-3-((5-amino-2-bromopyridin-4-yl)amino)cyclohexyl)carbamate (162 mg, 0.42 mmol) and trimethyl orthoformate (276 μL, 2.5 mmol) as a solution in toluene (3 mL) was sealed and heated to 120° C. for 16 h. Upon completion, the reaction mixture was concentrated in vacuo. The resulting residue was purified by Biotage Isolera (CH₂Cl₂/MeOH, up to 10% MeOH) to give the desired product. LCMS calculated for C₁₇H₂₄BrN₄O₂(M+H)⁺: m/z=395.1/397.1; found: 395.1/397.1.

### Step 4. 1-((1S,3R)-3-Aminocyclohexyl)-1H-imidazo[4,5-c]pyridine-6-carbonitrile

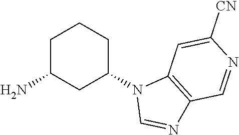 [SMILES: N#Cc1cc2c(cn1)ncn2[C@H]1CCC[C@@H](N)C1]

A microwave vial containing tert-butyl ((1R,3S)-3-(6-bromo-1H-imidazo[4,5-c]pyridin-1-yl)cyclohexyl)carbamate (122 mg, 0.31 mmol), Zn(CN)₂ (72 mg, 0.62 mmol), zinc powder (81 mg, 1.2 mmol), and Pd(dppf)Cl₂ CH₂Cl₂(38 mg, 0.46 mmol) was evacuated and backfilled with nitrogen three times, followed by the addition of DMF (2 mL). The vessel was irradiated at 120° C. for 1 h using a Biotage Initator+ Microwave Synthesizer. After cooling to rt, the reaction mixture was filtered over Celite and washed with CH₂Cl₂, followed by concentration of the filtrate in vacuo to remove CH₂Cl₂. The remaining DMF solution was diluted with MeOH (2 mL) and poured into ice water, and the resulting solid was collected by filtration. The solid was washed with water, hexanes, and left to dry under vacuum. The residue was then purified by Biotage Isolera (CH₂Cl₂/MeOH, up to 10% MeOH). The purified material was then dissolved in MeOH (2 mL) and 4N HCl/dioxane (1 mL) and left to stir at rt for 1 h. Upon completion, the reaction mixture was concentrated in vacuo. The obtained crude product was used in the next step without further purification. LCMS calculated for C₁₃H₁₆N₅(M+H)⁺: m/z=242.1; found: 242.2.

### Step 5. 1-((1S,3R)-3-((5-Cyano-4-(1-(2,2-difluoroethyl)-1H-pyrazol-4-yl)pyrimidin-2-yl)amino)cyclohexyl)-1H-imidazo[4,5-c]pyridine-6-carbonitrile

To a solution of 1-((1S,3R)-3-aminocyclohexyl)-1H-imidazo[4,5-c]pyridine-6-carbonitrile (18 mg, 0.075 mmol) in n-BuOH (1 mL) was added 4-(1-(2,2-difluoroethyl)-1H-pyrazol-4-yl)-2-(methylsulfonyl)pyrimidine-5-carbonitrile (Intermediate 1, 23 mg, 0.075 mmol) and DIPEA (78 μL, 0.45 mmol). The reaction vial was sealed and heated to 60° C. for 15 min. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₁F₂N₁₀ (M+H)⁺: m/z=475.2; found: 475.2.

### Example 47. 3-((1S,3R)-3-((5-Cyano-4-(1-(2,2-difluoroethyl)-1H-pyrazol-4-yl)pyrimidin-2-yl)amino)cyclohexyl)-3H-imidazo[4,5-b]pyridine-5-carbonitrile

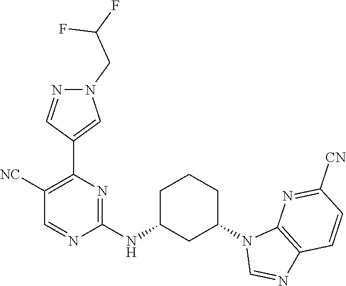 [SMILES: N#Cc1ccc2ncn([C@H]3CCC[C@@H](Nc4ncc(C#N)c(-c5cnn(CC(F)F)c5)n4)C3)c2n1]

This compound was prepared according to the procedures described in Example 46, with 6-bromo-2-chloro-3-nitropyridine replacing 2-bromo-4-chloro-5-nitropyridine in Step 1. LCMS calculated for C₂₃H₂₁F₂N₁₀ (M+H)⁺: m/z=475.2; found: 475.2.

### Example 48. 1-((1S,3R)-3-((5-Cyano-4-(1-(2,2-difluoroethyl)-1H-pyrazol-4-yl)pyrimidin-2-yl)amino)cyclohexyl)-4-methyl-1H-imidazo[4,5-c]pyridine-7-carbonitrile

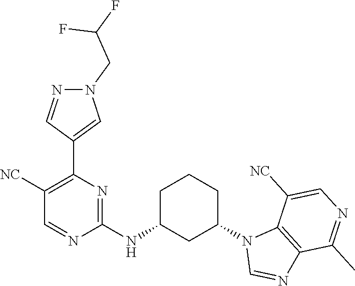 [SMILES: Cc1ncc(C#N)c2c1ncn2[C@H]1CCC[C@@H](Nc2ncc(C#N)c(-c3cnn(CC(F)F)c3)n2)C1]

### Step 1. tert-Butyl ((1R,3S)-3-((5-bromo-2-methyl-3-nitropyridin-4-yl)amino)cyclohexyl)carbamate

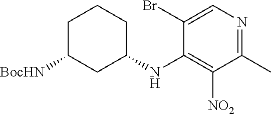 [SMILES: Cc1ncc(Br)c(N[C@H]2CCC[C@@H](NC(=O)OC(C)(C)C)C2)c1[N+](=O)[O-]]

A mixture of tert-butyl ((1R,3S)-3-aminocyclohexyl)carbamate (85 mg, 0.40 mmol), 5-bromo-4-chloro-2-methyl-3-nitropyridine (100 mg, 0.40 mmol), and DIPEA (104 μL, 0.60 mmol) in EtOH (4 mL) was heated to 80° C. and stirred for 2 h. Upon completion, the reaction mixture was cooled to rt and concentrated in vacuo. The crude residue was purified by Biotage Isolera (CH₂Cl₂/MeOH, up to 10% MeOH) to give the desired product as yellow solid. LCMS calculated for C₁₇H₂₆BrN₄O₄ (M+H)⁺: m/z=429.1/431.1; found: 429.1/431.1.

### Step 2. tert-Butyl ((1R,3S)-3-((3-amino-5-bromo-2-methylpyridin-4-yl)amino)cyclohexyl)carbamate

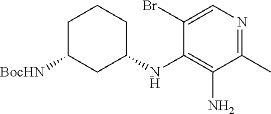 [SMILES: Cc1ncc(Br)c(N[C@H]2CCC[C@@H](NC(=O)OC(C)(C)C)C2)c1N]

To a vial containing tert-butyl ((1R,3S)-3-((5-bromo-2-methyl-3-nitropyridin-4-yl)amino)cyclohexyl)carbamate (171 mg, 0.40 mmol) as a solution in THE (1 mL), MeOH (1 mL), and water (1 mL) was added iron powder (111 mg, 2.0 mmol) and ammonium chloride (128 mg, 2.4 mmol). The vial was sealed and heated to 60° C. for 1 h. After cooling to rt, the reaction mixture was diluted with CH₂Cl₂(5 mL) and filtered over Celite. The filtrate was concentrated in vacuo and the resulting crude residue was used in the next step with no further purification. LCMS calculated for C₁₇H₂₈BrN₄O₂(M+H)+: m/z=399.1/401.1; found: 399.2/401.2.

### Step 3. tert-Butyl ((1R,3S)-3-(7-bromo-4-methyl-1H-imidazo[4,5-c]pyridin-1-yl)cyclohexyl)carbamate

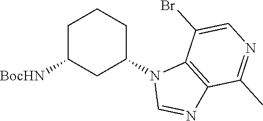 [SMILES: Cc1ncc(Br)c2c1ncn2[C@H]1CCC[C@@H](NC(=O)OC(C)(C)C)C1]

A vial containing tert-butyl ((1R,3S)-3-((3-amino-5-bromo-2-methylpyridin-4-yl)amino)cyclohexyl)carbamate (159 mg, 0.40 mmol) and trimethyl orthoformate (264 μL, 2.4 mmol) as a solution in toluene (3 mL) was sealed and heated to 120° C. for 16 h. Upon completion, the reaction mixture was concentrated in vacuo. The resulting residue was purified by Biotage Isolera (CH₂Cl₂/MeOH, up to 10% MeOH) to give the desired product. LCMS calculated for C₁₈H₂₆BrN₄O₂(M+H)⁺: m/z=409.1/411.1; found: 409.1/411.1.

### Step 4. 1-((1S,3R)-3-Aminocyclohexyl)-4-methyl-1H-imidazo[4,5-c]pyridine-7-carbonitrile

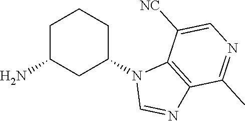 [SMILES: Cc1ncc(C#N)c2c1ncn2[C@H]1CCC[C@@H](N)C1]

A microwave vial containing tert-butyl ((1R,3S)-3-(7-bromo-4-methyl-1H-imidazo[4,5-c]pyridin-1-yl)cyclohexyl)carbamate (50 mg, 0.12 mmol), Zn(CN)₂ (29 mg, 0.24 mmol), zinc powder (32 mg, 0.50 mmol), and Pd(dppf)Cl₂ CH₂Cl₂(15 mg, 0.02 mmol) was evacuated and backfilled with nitrogen three times, followed by the addition of DMF (1 mL). The vessel was irradiated at 120° C. for 1 h using a Biotage Initator+ Microwave Synthesizer. After cooling to rt, the reaction mixture was filtered over Celite and washed with CH₂Cl₂, followed by concentration of the filtrate in vacuo to remove CH₂Cl₂. The remaining DMF solution was diluted with MeOH (1 mL) and poured into ice water, and the resulting solid was collected by filtration. The solid was washed with water, hexanes, and left to dry under vacuum. The residue was then purified by Biotage Isolera (CH₂Cl₂/MeOH, up to 10% MeOH). The purified material was then dissolved in MeOH (2 mL) and 4N HCl/dioxane (2 mL) and left to stir at rt for 1 h. Upon completion, the reaction mixture was concentrated in vacuo. The obtained crude product was used in the next step without further purification. LCMS calculated for C₁₄H₁₈N₅(M+H)⁺: m/z=256.2; found: 256.2.

### Step 5. 1-((1S,3R)-3-((5-Cyano-4-(1-(2,2-difluoroethyl)-1H-pyrazol-4-yl)pyrimidin-2-yl)amino)cyclohexyl)-4-methyl-1H-imidazo[4,5-c]pyridine-7-carbonitrile

To a solution of 1-((1S,3R)-3-aminocyclohexyl)-4-methyl-1H-imidazo[4,5-c]pyridine-7-carbonitrile (13 mg, 0.05 mmol) in n-BuOH (1 mL) was added 4-(1-(2,2-difluoroethyl)-1H-pyrazol-4-yl)-2-(methylsulfonyl)pyrimidine-5-carbonitrile (Intermediate 1, 16 mg, 0.05 mmol) and DIPEA (54 μL, 0.31 mmol). The reaction vial was sealed and heated to 60° C. for 15 min. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₄H₂₃F₂N₁₀ (M+H)⁺: m/z=489.2; found: 489.2. ¹H NMR (500 MHz, DMSO-d₆, mixture of tautomers) δ 8.78 (s, 0.55H), 8.76 (s, 1H), 8.75 (s, 0.45H), 8.73 (s, 0.45H), 8.64 (s, 0.55H), 8.63 (s, 0.55H), 8.55 (s, 0.45H), 8.40 (dd, J=11.6, 7.7 Hz, 1H), 8.34 (s, 0.55H), 8.21 (s, 0.45H), 6.56-6.29 (m, 1H), 4.93-4.72 (m, 3H), 4.20-4.05 (m, 1H), 2.82 (s, 3H), 2.48-2.42 (m, 1H), 2.24-2.14 (m, 1H), 2.09-1.84 (m, 4H), 1.68-1.39 (m, 2H).

### Example 49. 1-((1S,3R)-3-((5-Cyano-4-(1-(2,2-difluoroethyl)-1H-pyrazol-4-yl)pyrimidin-2-yl)amino)cyclohexyl)-5-fluoro-1H-benzo[d]imidazole-7-carbonitrile

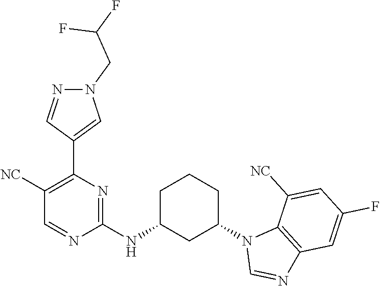 [SMILES: N#Cc1cnc(N[C@@H]2CCC[C@H](n3cnc4cc(F)cc(C#N)c43)C2)nc1-c1cnn(CC(F)F)c1]

This compound was prepared according to the procedures described in Example 46, with 1-bromo-2,5-difluoro-3-nitrobenzene replacing 2-bromo-4-chloro-5-nitropyridine in Step 1. LCMS calculated for C₂₄H₂₁F₃N₉ (M+H)⁺: m/z=492.2; found: 492.3.

### Example 50. 1-((1S,3R)-3-((5-Cyano-4-(1-(2,2-difluoroethyl)-1H-pyrazol-4-yl)pyrimidin-2-yl)amino)cyclohexyl)-1H-benzo[d]imidazole-5,7-dicarbonitrile

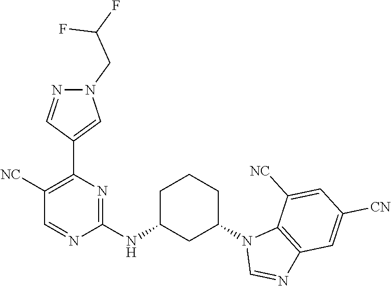 [SMILES: N#Cc1cc(C#N)c2c(c1)ncn2[C@H]1CCC[C@@H](Nc2ncc(C#N)c(-c3cnn(CC(F)F)c3)n2)C1]

This compound was prepared according to the procedures described in Example 46, with 1,5-dibromo-2-fluoro-3-nitrobenzene replacing 2-bromo-4-chloro-5-nitropyridine in Step 1. LCMS calculated for C₂₅H₂₁F₂N₁₀ (M+H)⁺: m/z=499.2; found: 499.3.

### Example 51. 1-((1S,3R)-3-((4-(1-(2,2-Difluoroethyl)-1H-pyrazol-4-yl)-5-(trifluoromethyl)pyrimidin-2-yl)amino)cyclohexyl)-5-(trifluoromethyl)-1H-benzo[d]imidazole-7-carbonitrile

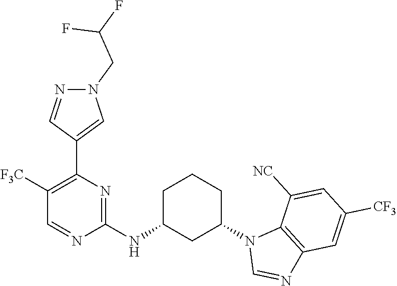 [SMILES: N#Cc1cc(C(F)(F)F)cc2ncn([C@H]3CCC[C@@H](Nc4ncc(C(F)(F)F)c(-c5cnn(CC(F)F)c5)n4)C3)c12]

### Step 1. 1-((1S,3R)-3-Aminocyclohexyl)-5-(trifluoromethyl)-1H-benzo[d]imidazole-7-carbonitrile

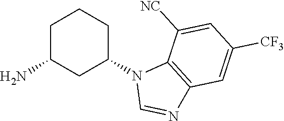 [SMILES: N#Cc1cc(C(F)(F)F)cc2ncn([C@H]3CCC[C@@H](N)C3)c12]

This compound was prepared according to the procedures described in Example 46, Steps 1-4, with 1-bromo-2-fluoro-3-nitro-5-(trifluoromethyl)benzene replacing 2-bromo-4-chloro-5-nitropyridine in Step 1. LCMS calculated for C₁₅H₁₆F₃N₄ (M+H)⁺: m/z=309.1; found: 309.1.

### Step 2. 1-((1S,3R)-3-((4-(1-(2,2-Difluoroethyl)-1H-pyrazol-4-yl)-5-(trifluoromethyl)pyrimidin-2-yl)amino)cyclohexyl)-5-(trifluoromethyl)-1H-benzo[d]imidazole-7-carbonitrile

To a solution of 1-((1S,3R)-3-aminocyclohexyl)-5-(trifluoromethyl)-1H-benzo[d]imidazole-7-carbonitrile (12 mg, 0.038 mmol) in EtOH (500 μL) was added 2-chloro-4-(1-(2,2-difluoroethyl)-1H-pyrazol-4-yl)-5-(trifluoromethyl)pyrimidine (12 mg, 0.038 mmol, Example 15, Step 2) and DIPEA (40 μL, 0.23 mmol). The reaction vial was sealed and heated to 60° C. for 1 h. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min).LCMS calculated for C₂₅H₂₁F₈N₈ (M+H)⁺: m/z=585.2; found: 585.2.

### Example 52. 1-((1S,3R)-3-((4-(1-(2,2-Difluoroethyl)-1H-pyrazol-4-yl)-5-(trifluoromethyl)pyrimidin-2-yl)amino)cyclohexyl)-1H-imidazo[4,5-c]pyridine-7-carbonitrile

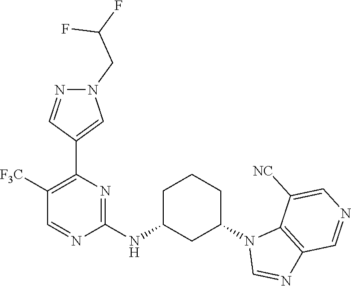 [SMILES: N#Cc1cncc2ncn([C@H]3CCC[C@@H](Nc4ncc(C(F)(F)F)c(-c5cnn(CC(F)F)c5)n4)C3)c12]

To a solution of 1-((1S,3R)-3-aminocyclohexyl)-1H-imidazo[4,5-c]pyridine-7-carbonitrile (9.3 mg, 0.038 mmol, Example 31, Step 4) in EtOH (500 μL) was added 2-chloro-4-(1-(2,2-difluoroethyl)-1H-pyrazol-4-yl)-5-(trifluoromethyl)pyrimidine (12 mg, 0.038 mmol, Example 15, Step 2) and DIPEA (40 μL, 0.23 mmol). The reaction vial was sealed and heated to 60° C. for 1 h. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₁F₅N₉ (M+H)⁺: m/z=518.2; found: 518.2. ¹H NMR (600 MHz, DMSO-d₆, mixture of tautomers) δ 9.27 (s, 1H), 8.87 (s, 1H), 8.83 (s, 0.55H), 8.80 (s, 0.45H), 8.62 (s, 0.45H), 8.55 (s, 0.55H), 8.36 (s, 0.55H), 8.28 (s, 0.45H), 8.18-8.11 (m, 1H), 8.11 (s, 0.55H), 7.98 (s, 0.45H), 6.52-6.29 (m, 1H), 4.93-4.79 (m, 1H), 4.79-4.71 (m, 2H), 4.18-4.05 (m, 1H), 2.25-2.17 (m, 1H), 2.10-1.85 (m, 5H), 1.67-1.51 (m, 1H), 1.51-1.40 (m, 1H).

### Example 53. 6-Cyano-3-((1S,3R)-3-((5-cyano-4-(1-(2,2-difluoroethyl)-1H-pyrazol-4-yl)pyrimidin-2-yl)amino)cyclohexyl)-3H-imidazo[4,5-b]pyridine 4-oxide

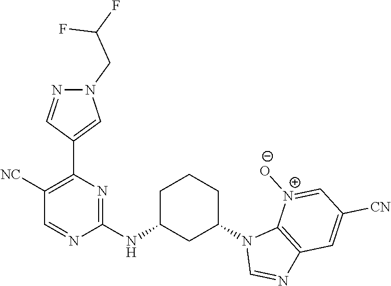 [SMILES: N#Cc1cc2ncn([C@H]3CCC[C@@H](Nc4ncc(C#N)c(-c5cnn(CC(F)F)c5)n4)C3)c2[n+]([O-])c1]

### Step 1. 3-((1S,3R)-3-Aminocyclohexyl)-6-cyano-3H-imidazo[4,5-b]pyridine 4-oxide

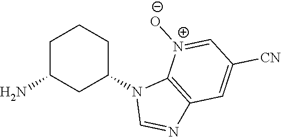 [SMILES: N#Cc1cc2ncn([C@H]3CCC[C@@H](N)C3)c2[n+]([O-])c1]

To a vial containing tert-butyl ((1R,3S)-3-(6-cyano-3H-imidazo[4,5-b]pyridin-3-yl)cyclohexyl)carbamate (10 mg, 0.03 mmol, Example 9A, Step 3) as a solution in CHCl₃ (500 μL) was added m-CPBA (13 mg, 0.06 mmol) and left to stir at rt for 2 h. The reaction mixture was concentrated in vacuo and the resulting residue was purified by Teledyne ISCO CombiFlash (CH₂Cl₂/MeOH, up to 80% MeOH). The purified material was then dissolved in CH₂Cl₂ (1 mL) and TFA (1 mL) and left to stir at rt for 1 h. Upon completion, the reaction mixture was concentrated in vacuo. The obtained crude product was used in the next step without further purification. LCMS calculated for C₁₃H₁₆N₅O (M+H)⁺: m/z=258.1; found: 258.2.

### Step 2. 6-Cyano-3-((1S,3R)-3-((5-Cyano-4-(1-(2,2-difluoroethyl)-1H-pyrazol-4-yl)pyrimidin-2-yl)amino)cyclohexyl)-3H-imidazo[4,5-b]pyridine 4-oxide

To a solution of 3-((1S,3R)-3-aminocyclohexyl)-6-cyano-3H-imidazo[4,5-b]pyridine 4-oxide (11 mg, 0.045 mmol) in n-BuOH (500 μL) was added 4-(1-(2,2-difluoroethyl)-1H-pyrazol-4-yl)-2-(methylsulfonyl)pyrimidine-5-carbonitrile (Intermediate 1, 14 mg, 0.045 mmol) and DIPEA (47 μL, 0.27 mmol). The reaction vial was sealed and heated to 60° C. for 15 min. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₁F₂N₁₀O (M+H)⁺: m/z=491.2; found: 491.2.

### Example 54. 4-(1-(2,2-Difluoroethyl)-1H-pyrazol-4-yl)-2-(((1R,3S)-3-(5-fluoro-7-methyl-1H-benzo[d]imidazol-1-yl)cyclohexyl)amino)pyrimidine-5-carbonitrile

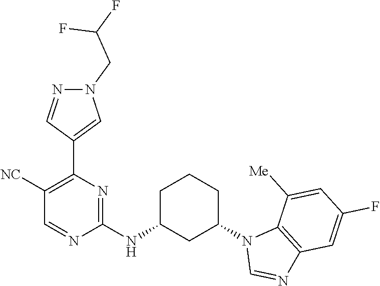 [SMILES: Cc1cc(F)cc2ncn([C@H]3CCC[C@@H](Nc4ncc(C#N)c(-c5cnn(CC(F)F)c5)n4)C3)c12]

### Step 1. (1R,3S)-3-(5-Fluoro-7-methyl-1H-benzo[d]imidazol-1-yl)cyclohexan-1-amine

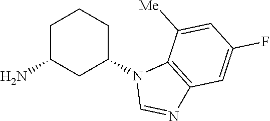 [SMILES: Cc1cc(F)cc2ncn([C@H]3CCC[C@@H](N)C3)c12]

A vial containing a mixture of tert-butyl ((1R,3S)-3-(7-chloro-5-fluoro-1H-benzo[d]imidazol-1-yl)cyclohexyl)carbamate (50 mg, 0.14 mmol, Example 36, Step 3), XPhos Pd G2 (11 mg, 14 mol) and tripotassium phosphate (87 mg, 0.41 mmol) was evacuated and backfilled with nitrogen three times, followed by the addition of 1,4-dioxane (1 mL), water (200 μL), and trimethylboroxine (38 μL, 0.27 mmol). The vial was sealed and the reaction was stirred at 80° C. for 1 h before being cooled to rt, diluted with CH₂Cl₂ (5 mL), and filtered through a plug of Celite. The filtrate was concentrated in vacuo, and the resulting crude residue was used in the next step without further purification. LCMS calculated for C₁₄H₁₉FN₃ (M+H)⁺: m/z=248.2; found: 248.2.

### Step 2. 4-(1-(2,2-Difluoroethyl)-1H-pyrazol-4-yl)-2-(((R,3S)-3-(5-fluoro-7-methyl-1H-benzo[d]imidazol-1-yl)cyclohexyl)amino)pyrimidine-5-carbonitrile

To a solution of (1R,3S)-3-(5-fluoro-7-methyl-1H-benzo[d]imidazol-1-yl)cyclohexan-1-amine (11 mg, 0.045 mmol) in n-BuOH (1 mL) was added 4-(1-(2,2-difluoroethyl)-1H-pyrazol-4-yl)-2-(methylsulfonyl)pyrimidine-5-carbonitrile (Intermediate 1, 14 mg, 0.045 mmol) and DIPEA (47 μL, 0.27 mmol). The reaction vial was sealed and heated to 60° C. for 15 min. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₄H₂₄F₃N₈ (M+H)⁺: m/z=481.2; found: 481.3. ¹H NMR (500 MHz, DMSO-d₆, mixture of tautomers) δ 9.10 (s, 0.6H), 9.07 (s, 0.4H), 8.75 (s, 0.4H), 8.65 (s, 0.6H), 8.64 (s, 0.6H), 8.54 (s, 0.4H), 8.40-8.36 (m, 1H), 8.36 (s, 0.6H), 8.21 (s, 0.4H), 7.45-7.39 (m, 1H), 7.19-7.09 (m, 1H), 6.59-6.29 (m, 1H), 4.96-4.76 (m, 3H), 4.31-4.09 (m, 1H), 2.80 (s, 1.2H), 2.77 (s, 1.8H), 2.49-2.43 (m, 1H), 2.20 (t, J=13.4 Hz, 1H), 2.08-1.85 (m, 3H), 1.85-1.60 (m, 2H), 1.53-1.40 (m, 1H).

### Example 55. 1-((1S,3R)-3-((5-Cyano-4-(1-(2,2-difluoroethyl)-1H-pyrazol-4-yl)pyrimidin-2-yl)amino)cyclohexyl)-7-methyl-1H-benzo[d]imidazole-5-carbonitrile

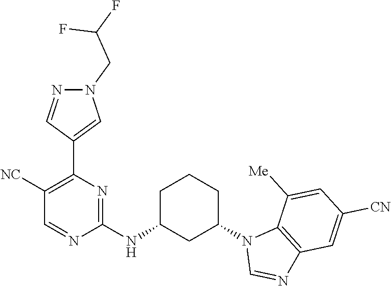 [SMILES: Cc1cc(C#N)cc2ncn([C@H]3CCC[C@@H](Nc4ncc(C#N)c(-c5cnn(CC(F)F)c5)n4)C3)c12]

### Step 1. tert-Butyl ((1R,3S)-3-(5-bromo-7-chloro-1H-benzo[d]imidazol-1-yl)cyclohexyl)carbamate

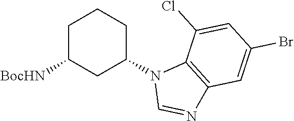 [SMILES: CC(C)(C)OC(=O)N[C@@H]1CCC[C@H](n2cnc3cc(Br)cc(Cl)c32)C1]

This compound was prepared according to the procedures described in Example 3, Steps 1-3, with 5-bromo-1-chloro-2-fluoro-3-nitrobenzene replacing 1-fluoro-2-nitrobenzene in Step 1. LCMS calculated for C₁₈H₂₄BrClN₃O₂(M+H)⁺: m/z=428.1/430.1/432.1; found: 428.1/430.1/432.1.

### Step 2. tert-Butyl ((1R,3S)-3-(7-chloro-5-cyano-1H-benzo[d]imidazol-1-yl)cyclohexyl)carbamate

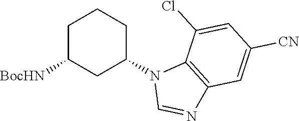 [SMILES: CC(C)(C)OC(=O)N[C@@H]1CCC[C@H](n2cnc3cc(C#N)cc(Cl)c32)C1]

A vial containing tert-butyl ((1R,3S)-3-(5-bromo-7-chloro-1H-benzo[d]imidazol-1-yl)cyclohexyl)carbamate (44 mg, 0.10 mmol), Zn(CN)₂ (10 mg, 0.08 mmol), Pd₂(dba)₃ (9.4 mg, 10 μmol) and 1,1′-bis(diphenylphosphino)ferrocene (11 mg, 21 μmol) was evacuated and backfilled with nitrogen three times, followed by the addition of DMF (1 mL). The reaction vial was sealed and heated to 120° C. for 1 h. Upon completion, the reaction mixture was cooled to rt, poured into ice water, and the resulting solid was collected by filtration. The solid was washed with water, hexanes, and left to dry under vacuum. The resulting residue was purified by Teledyne ISCO CombiFlash (CH₂Cl₂/MeOH, up to 10% MeOH) to give the desired product. LCMS calculated for C₁₉H₂₄ClN₄O₂(M+H)⁺: m/z=375.2/377.2; found: 375.2/377.2.

### Step 3. 1-((1S,3R)-3-Aminocyclohexyl)-7-methyl-1H-benzo[d]imidazole-5-carbonitrile

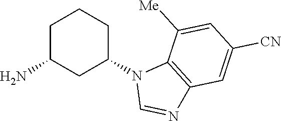 [SMILES: Cc1cc(C#N)cc2ncn([C@H]3CCC[C@@H](N)C3)c12]

A vial containing a mixture of tert-butyl ((1R,3S)-3-(7-chloro-5-cyano-1H-benzo[d]imidazol-1-yl)cyclohexyl)carbamate (23 mg, 0.06 mmol), XPhos Pd G2 (5 mg, 6 mol) and tripotassium phosphate (39 mg, 0.18 mmol) was evacuated and backfilled with nitrogen three times, followed by the addition of 1,4-dioxane (1 mL), water (250 μL), and trimethylboroxine (26 μL, 0.18 mmol). The vial was sealed and the reaction was stirred at 100° C. for 1 h before being cooled to rt, diluted with CH₂Cl₂ (5 mL), and filtered through a plug of Celite. The filtrate was concentrated in vacuo. The resulting crude residue was dissolved in MeOH (1 mL) and 4N HCl/dioxane (1 mL) and left to stir at rt for 1 h. Upon completion, the reaction mixture was concentrated in vacuo. The obtained crude product was used in the next step without further purification. LCMS calculated for C₁₅H₁₉N₄(M+H)⁺: m/z=255.2; found: 255.2.

### Step 4. 1-((1S,3R)-3-((5-Cyano-4-(1-(2,2-difluoroethyl)-1H-pyrazol-4-yl)pyrimidin-2-yl)amino)cyclohexyl)-7-methyl-1H-benzo[d]imidazole-5-carbonitrile

To a solution of 1-((1S,3R)-3-aminocyclohexyl)-7-methyl-1H-benzo[d]imidazole-5-carbonitrile (16 mg, 0.064 mmol) in n-BuOH (1 mL) was added 4-(1-(2,2-difluoroethyl)-1H-pyrazol-4-yl)-2-(methylsulfonyl)pyrimidine-5-carbonitrile (Intermediate 1, 20 mg, 0.064 mmol) and DIPEA (67 μL, 0.38 mmol). The reaction vial was sealed and heated to 60° C. for 15 min. Upon completion, the reaction mixture was cooled to rt, diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₅H₂₄F₂N₉ (M+H)⁺: m/z=488.2; found: 488.2.

### Example 56. 3-((1S,3R)-3-((4-(1-(2,2-Difluoroethyl)-1H-pyrazol-4-yl)-5-(1-methyl-1H-pyrazol-4-yl)pyrimidin-2-yl)amino)cyclohexyl)-3H-imidazo[4,5-b]pyridine-6-caronitrile

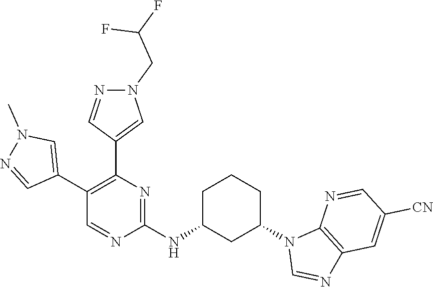 [SMILES: Cn1cc(-c2cnc(N[C@@H]3CCC[C@H](n4cnc5cc(C#N)cnc54)C3)nc2-c2cnn(CC(F)F)c2)cn1]

A mixture of 3-((1S,3R)-3-((5-chloro-4-(1-(2,2-difluoroethyl)-1H-pyrazol-4-yl)pyrimidin-2-yl)amino)cyclohexyl)-3H-imidazo[4,5-b]pyridine-6-carbonitrile (31 mg, 0.06 mmol, Example 14), 1-methyl-4-(4,4,5,5-tetramethyl-1,3,2-dioxaborolan-2-yl)-1H-pyrazole (15 g, 0.07 mmol), XPhos Pd G2 (51 mg, 0.06 mmol) and cesium carbonate (21 mg, 0.06 mmol) was suspended in MeCN (2 mL) and water (200 μL). The solution was purged with nitrogen for 2 min and the reaction was stirred at 80° C. After 2 h, the solution was cooled to rt and diluted with MeOH and filtered through a Silicycle SiliaPrep™ Thiol cartridge (Cat. #SPE-R51030B). The filtrate was then diluted with MeCN and purified by prep-LCMS (XBridge C₁₈ column, eluting with a gradient of acetonitrile/water containing 0.1% TFA, at flow rate of 60 mL/min). LCMS calculated for C₂₆H₂₆F₂N₁₁ (M+H)⁺: m/z=530.2; found: 530.3.

### Example 57. 3-((1S,3R)-3-((4-(1-(2,2-Difluoroethyl)-1H-pyrazol-4-yl)-5-(1H-pyrazol-4-yl)pyrimidin-2-yl)amino)cyclohexyl)-3H-imidazo[4,5-b]pyridine-6-carbonitrile

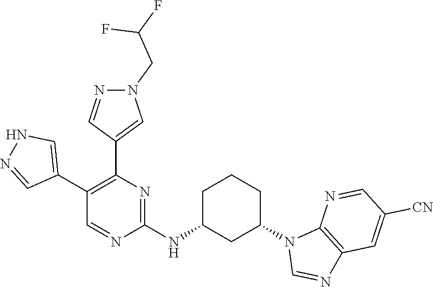 [SMILES: N#Cc1cnc2c(c1)ncn2[C@H]1CCC[C@@H](Nc2ncc(-c3cn[nH]c3)c(-c3cnn(CC(F)F)c3)n2)C1]

A mixture of 3-((1S,3R)-3-((5-chloro-4-(1-(2,2-difluoroethyl)-1H-pyrazol-4-yl)pyrimidin-2-yl)amino)cyclohexyl)-3H-imidazo[4,5-b]pyridine-6-carbonitrile (31 mg, 0.06 mmol, Example 14), tert-butyl 4-(4,4,5,5-tetramethyl-1,3,2-dioxaborolan-2-yl)-1H-pyrazole-1-carboxylate (21 mg, 0.07 mmol), XPhos Pd G2 (51 mg, 0.06 mmol) and cesium carbonate (21 mg, 0.06 mmol) was suspended in MeCN (2 mL) and water (200 μL). The solution was purged with nitrogen for 2 min, and the reaction was stirred at 80° C. After 2 h, LCMS indicated consumption of the starting material. The solution was cooled to rt and filtered through a pad of Celite. The filtrate was concentrated in vacuo and the resulting residue was dissolved in TFA and CH₂Cl₂. The solution was stirred at rt for 30 min and then concentrated in vacuo. The residue was dissolved in MeOH and filtered through a Silicycle SiliaPrep™ Thiol cartridge (Cat. #SPE-R51030B). The filtrate was then diluted in MeCN and purified by prep-LCMS (XBridge C₁₈ column, eluting with a gradient of acetonitrile/water containing 0.1% TFA, at flow rate of 60 mL/min). LCMS calculated for C₂₅H₂₄F₂Nₙ(M+H)⁺: m/z=516.2; found: 516.3.

### Example 58. 3-((1S,3R)-3-((5-Cyano-4-(1-(2-hydroxy-2-methylpropyl)-1H-pyrazol-4-yl)pyrimidin-2-yl)amino)cyclohexyl)-3H-imidazo[4,5-b]pyridine-6-carbonitrile

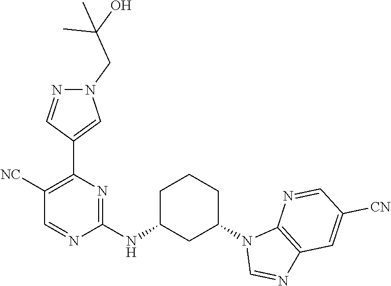 [SMILES: CC(C)(O)Cn1cc(-c2nc(N[C@@H]3CCC[C@H](n4cnc5cc(C#N)cnc54)C3)ncc2C#N)cn1]

This compound was prepared according to the procedures described in Example 9A, with 2-methyl-1-(4-(4,4,5,5-tetramethyl-1,3,2-dioxaborolan-2-yl)-1H-pyrazol-1-yl)propan-2-ol replacing 1-(2,2-difluoroethyl)-4-(4,4,5,5-tetramethyl-1,3,2-dioxaborolan-2-yl)-1H-pyrazole in Step 6. LCMS calculated for C₂₅H₂₇N₁₀O (M+H)⁺: m/z=483.2; found: 483.3.

### Example 59. 3-((1S,3R)-3-((4-(1-(Azetidin-3-yl)-1H-pyrazol-4-yl)-5-cyanopyrimidin-2-yl)amino)cyclohexyl)-3H-imidazo[4,5-b]pyridine-6-carbonitrile

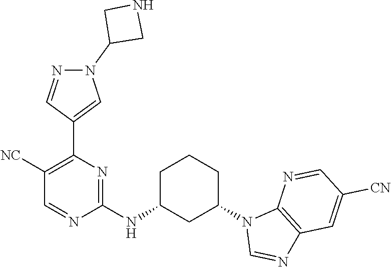 [SMILES: N#Cc1cnc2c(c1)ncn2[C@H]1CCC[C@@H](Nc2ncc(C#N)c(-c3cnn(C4CNC4)c3)n2)C1]

A vial containing 3-((1S,3R)-3-((4-chloro-5-cyanopyrimidin-2-yl)amino)cyclohexyl)-3H-imidazo[4,5-b]pyridine-6-carbonitrile (35 mg, 0.09 mmol, Example 9A, Step 5), tert-butyl 3-(4-(4,4,5,5-tetramethyl-1,3,2-dioxaborolan-2-yl)-1H-pyrazol-1-yl)azetidine-1-carboxylate (35 mg, 0.10 mmol), sodium carbonate (29 mg, 0.28 mmol), and Pd(dppf)Cl₂ CH₂Cl₂ (15 mg, 0.02 mmol) was evacuated and backfilled with nitrogen three times, followed by the addition of MeCN (1 mL) and water (200 μL). The vial was sealed and heated to 140° C. for 5 min. After cooling to rt, the mixture was diluted with MeCN, filtered through Celite and the filtrate was concentrated in vacuo. The obtained crude product was dissolved in CH₂Cl₂(1 mL) and TFA (1 mL) and left to stir at rt. After 1 h, all volatiles were removed in vacuo. The crude residue was dissolved in MeCN, and purified by prep-LCMS (XBridge C₁₈ column, eluting with a gradient of acetonitrile/water containing 0.1% TFA, at flow rate of 60 mL/min). LCMS calculated for C₂₄H₂₄N₁₁ (M+H)⁺: m/z=466.2; found: 466.3.

### Example 60. 3-((1S,3R)-3-((5-Cyano-4-(1-(2-morpholinoethyl)-1H-pyrazol-4-yl)pyrimidin-2-yl)amino)cyclohexyl)-3H-imidazo[4,5-b]pyridine-6-carbonitrile

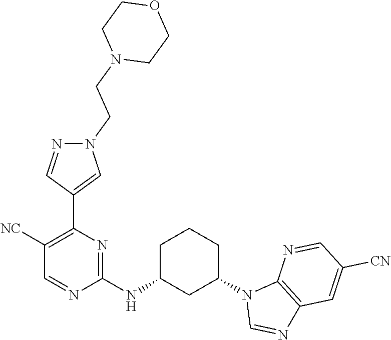 [SMILES: N#Cc1cnc2c(c1)ncn2[C@H]1CCC[C@@H](Nc2ncc(C#N)c(-c3cnn(CCN4CCOCC4)c3)n2)C1]

This compound was prepared according to the procedures described in Example 9A, with 4-(2-(4-(4,4,5,5-tetramethyl-1,3,2-dioxaborolan-2-yl)-1H-pyrazol-1-yl)ethyl)morpholine replacing 1-(2,2-difluoroethyl)-4-(4,4,5,5-tetramethyl-1,3,2-dioxaborolan-2-yl)-1H-pyrazole in Step 6. LCMS calculated for C₂₇H₃₀N₁₁O (M+H)⁺: m/z=524.3; found: 524.3.

### Example 61. 3-((1S,3R)-3-((5-Cyano-4-(1-methyl-1H-imidazol-4-yl)pyrimidin-2-yl)amino)cyclohexyl)-3H-imidazo[4,5-b]pyridine-6-carbonitrile

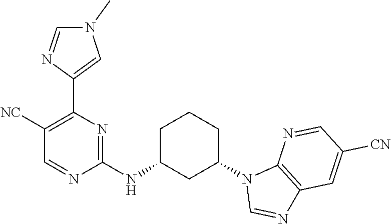 [SMILES: Cn1cnc(-c2nc(N[C@@H]3CCC[C@H](n4cnc5cc(C#N)cnc54)C3)ncc2C#N)c1]

This compound was prepared according to the procedures described in Example 9A, with 1-methyl-4-(4,4,5,5-tetramethyl-1,3,2-dioxaborolan-2-yl)-1H-imidazole replacing 1-(2,2-difluoroethyl)-4-(4,4,5,5-tetramethyl-1,3,2-dioxaborolan-2-yl)-1H-pyrazole in Step 6. LCMS calculated for C₂₂H₂₁N₁₀ (M+H)⁺: m/z=425.2; found: 425.3.

### Example 62. 3-((1S,3R)-3-((5-Cyano-4-(1-(2,2,2-trifluoroethyl)-1H-pyrazol-4-yl)pyrimidin-2-yl)amino)cyclohexyl)-3H-imidazo[4,5-b]pyridine-6-carbonitrile

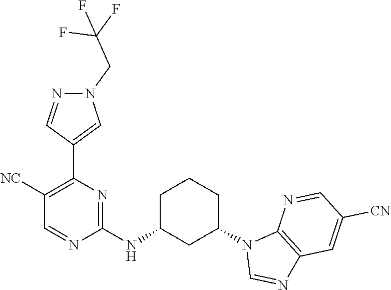 [SMILES: N#Cc1cnc2c(c1)ncn2[C@H]1CCC[C@@H](Nc2ncc(C#N)c(-c3cnn(CC(F)(F)F)c3)n2)C1]

This compound was prepared according to the procedures described in Example 9A, with 4-(4,4,5,5-tetramethyl-1,3,2-dioxaborolan-2-yl)-1-(2,2,2-trifluoroethyl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₃H₂₀F₃N₁₀ (M+H)⁺: m/z=493.2; found: 493.3.

### Example 63. 3-((1S,3R)-3-((5-Cyano-4-(1-(tetrahydro-2H-pyran-4-yl)-1H-pyrazol-4-yl)pyrimidin-2-yl)amino)cyclohexyl)-3H-imidazo[4,5-b]pyridine-6-carbonitrile

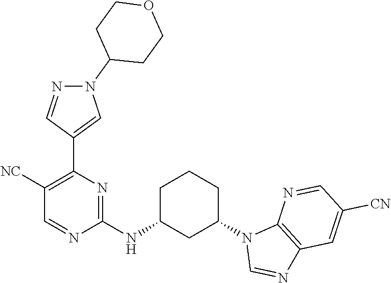 [SMILES: N#Cc1cnc2c(c1)ncn2[C@H]1CCC[C@@H](Nc2ncc(C#N)c(-c3cnn(C4CCOCC4)c3)n2)C1]

This compound was prepared according to the procedures described in Example 9A, with 1-(tetrahydro-2H-pyran-4-yl)-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₆H₂₇N₁₀O (M+H)⁺: m/z=495.2; found: 495.2.

### Example 64. 3-((1S,3R)-3-((5-Cyano-4-(1-methyl-1H-pyrazol-4-yl)pyrimidin-2-yl)amino)cyclohexyl)-3H-imidazo[4,5-b]pyridine-6-carbonitrile

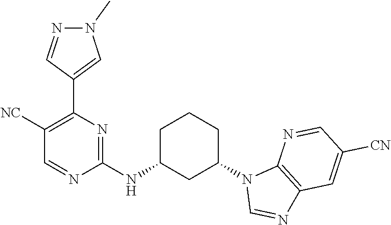 [SMILES: Cn1cc(-c2nc(N[C@@H]3CCC[C@H](n4cnc5cc(C#N)cnc54)C3)ncc2C#N)cn1]

This compound was prepared according to the procedures described in Example 9A, with 1-methyl-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₂H₂₁N₁₀ (M+H)⁺: m/z=425.2; found: 425.3.

### Example 65. 3-((1S,3R)-3-((5-Cyano-4-(1-phenyl-1H-pyrazol-4-yl)pyrimidin-2-yl)amino)cyclohexyl)-3H-imidazo[4,5-b]pyridine-6-carbonitrile

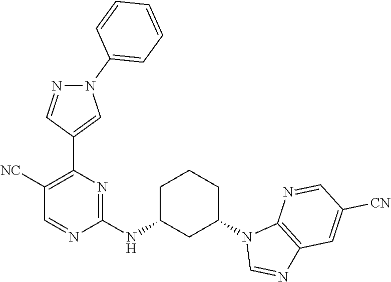 [SMILES: N#Cc1cnc2c(c1)ncn2[C@H]1CCC[C@@H](Nc2ncc(C#N)c(-c3cnn(-c4ccccc4)c3)n2)C1]

This compound was prepared according to the procedures described in Example 9A, with 1-phenyl-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₇H₂₃N₁₀ (M+H)⁺: m/z=487.2; found: 487.3.

### Example 66. 3-((1S,3R)-3-((5-Cyano-4-(1-(oxetan-3-yl)-1H-pyrazol-4-yl)pyrimidin-2-yl)amino)cyclohexyl)-3H-imidazo[4,5-b]pyridine-6-carbonitrile

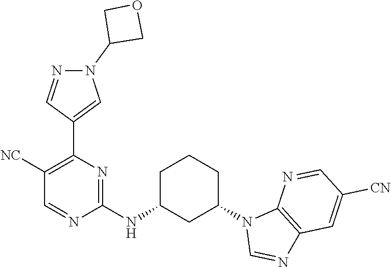 [SMILES: N#Cc1cnc2c(c1)ncn2[C@H]1CCC[C@@H](Nc2ncc(C#N)c(-c3cnn(C4COC4)c3)n2)C1]

A mixture of 1-((1S,3R)-3-((4-chloro-5-cyanopyrimidin-2-yl)amino)cyclohexyl)-1H-benzo[d]imidazole-5-carbonitrile (35 mg, 0.09 mmol, Example 9A, Step 5), 1-(oxetan-3-yl)-4-(4,4,5,5-tetramethyl-1,3,2-dioxaborolan-2-yl)-1H-pyrazole (25 mg, 0.10 mmol), and sodium carbonate (29 mg, 0.28 mmol) was dissolved in MeCN (1 mL) and water (200 μL). The solution was purged with nitrogen for 2 min, followed by addition of Pd(dppf)Cl₂ CH₂Cl₂ (15 mg, 0.02 mmol). The vial was sealed and the reaction was stirred at 140° C. for 5 min before being cooled to rt, diluted with CH₂Cl₂ (5 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₄H₂₃N₁₀O (M+H)⁺: m/z=467.2; found: 467.3.

### Example 67. 3-((1S,3R)-3-((5-Cyano-4-(1-(2-hydroxypropyl)-1H-pyrazol-4-yl)pyrimidin-2-yl)amino)cyclohexyl)-3H-imidazo[4,5-b]pyridine-6-carbonitrile

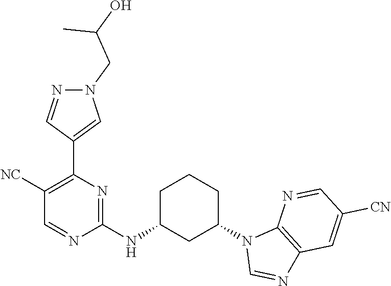 [SMILES: CC(O)Cn1cc(-c2nc(N[C@@H]3CCC[C@H](n4cnc5cc(C#N)cnc54)C3)ncc2C#N)cn1]

A mixture of 1-((1S,3R)-3-((4-chloro-5-cyanopyrimidin-2-yl)amino)cyclohexyl)-1H-benzo[d]imidazole-5-carbonitrile (35 mg, 0.09 mmol, Example 9A, Step 5), 1-(4-(4,4,5,5-tetramethyl-1,3,2-dioxaborolan-2-yl)-1H-pyrazol-1-yl)propan-2-ol (26 mg, 0.10 mmol), and sodium carbonate (29 mg, 0.28 mmol) was dissolved in MeCN (1 mL) and water (200 μL). The solution was purged with nitrogen for 2 min, followed by addition of Pd(dppf)Cl₂ CH₂Cl₂ (15 mg, 0.02 mmol). The vial was sealed and the reaction was stirred at 140° C. for 5 min before being cooled to rt, diluted with CH₂Cl₂ (5 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₄H₂₅N₁₀O (M+H)⁺: m/z=469.2; found: 469.3. ¹H NMR (500 MHz, DMSO-d₆, mixture of tautomers) δ 8.84-8.79 (m, 2H), 8.72-8.67 (m, 1.45H), 8.60 (s, 0.55H), 8.53 (s, 0.55H), 8.42 (s, 0.45H), 8.35 (s, 0.55H), 8.32-8.26 (m, 1H), 8.14 (s, 0.45H), 4.87-4.69 (m, 1H), 4.23-4.05 (m, 3H), 4.05-3.95 (m, 1H), 2.58-2.42 (m, 0.55H), 2.38-2.30 (m, 0.45H), 2.14-1.88 (m, 5H), 1.74-1.53 (m, 1H), 1.53-1.41 (m, 1H), 1.13-1.03 (m, 3H).

### Example 68. 3-((1S,3R)-3-((5-Cyano-4-(1-(2-hydroxyethyl)-1H-pyrazol-4-yl)pyrimidin-2-yl)amino)cyclohexyl)-3H-imidazo[4,5-b]pyridine-6-carbonitrile

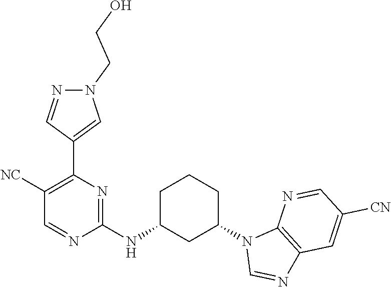 [SMILES: N#Cc1cnc2c(c1)ncn2[C@H]1CCC[C@@H](Nc2ncc(C#N)c(-c3cnn(CCO)c3)n2)C1]

A mixture of 1-((1S,3R)-3-((4-chloro-5-cyanopyrimidin-2-yl)amino)cyclohexyl)-1H-benzo[d]imidazole-5-carbonitrile (35 mg, 0.09 mmol, Example 9A, Step 5), with 2-(4-(4,4,5,5-tetramethyl-1,3,2-dioxaborolan-2-yl)-1H-pyrazol-1-yl)ethan-1-ol (22 mg, 0.09 mmol), and sodium carbonate (29 mg, 0.28 mmol) was dissolved in MeCN (1 mL) and water (200 μL). The solution was purged with nitrogen for 2 min, followed by addition of Pd(dppf)Cl₂ CH₂Cl₂ (15 mg, 0.02 mmol). The vial was sealed and the reaction was stirred at 140° C. for 5 min before being cooled to rt, diluted with CH₂Cl₂ (5 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₃N₁₀O (M+H)⁺: m/z=455.2; found: 455.3. ¹H NMR (500 MHz, DMSO-d₆, mixture of tautomers) δ 8.84-8.79 (m, 2H), 8.72-8.68 (m, 1.45H), 8.60 (s, 0.55H), 8.55 (s, 0.55H), 8.44 (s, 0.45H), 8.35 (s, 0.55H), 8.30 (d, J=7.8 Hz, 1H), 8.16 (s, 0.45H), 4.87-4.69 (m, 1H), 4.31-4.22 (m, 2H), 4.22-4.07 (m, 1H), 3.81-3.72 (m, 2H), 2.48-2.40 (m, 0.55H), 2.38-2.30 (m, 0.45H), 2.15-1.90 (m, 5H), 1.74-1.62 (m, 0.55H), 1.62-1.53 (m, 0.45H), 1.53-1.40 (m, 1H).

### Example 69. 3-((1S,3R)-3-((5-Cyano-4-(1-(cyclopropylmethyl)-1H-pyrazol-4-yl)pyrimidin-2-yl)amino)cyclohexyl)-3H-imidazo[4,5-b]pyridine-6-carbonitrile

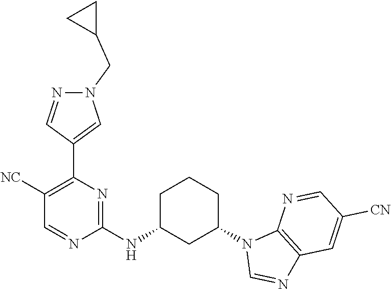 [SMILES: N#Cc1cnc2c(c1)ncn2[C@H]1CCC[C@@H](Nc2ncc(C#N)c(-c3cnn(CC4CC4)c3)n2)C1]

This compound was prepared according to the procedures described in Example 9A, with 1-(cyclopropylmethyl)-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₅H₂₅N₁₀ (M+H)⁺: m/z=465.2; found: 465.3.

### Example 70. 3-((1S,3R)-3-((5-Cyano-4-(1-(2-fluoroethyl)-1H-pyrazol-4-yl)pyrimidin-2-yl)amino)cyclohexyl)-3H-imidazo[4,5-b]pyridine-6-carbonitrile

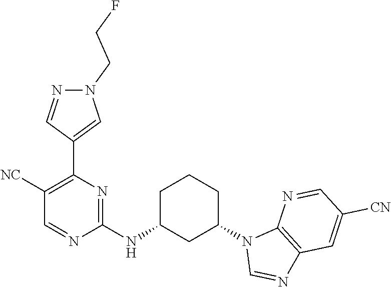 [SMILES: N#Cc1cnc2c(c1)ncn2[C@H]1CCC[C@@H](Nc2ncc(C#N)c(-c3cnn(CCF)c3)n2)C1]

A mixture of 1-((1S,3R)-3-((4-chloro-5-cyanopyrimidin-2-yl)amino)cyclohexyl)-1H-benzo[d]imidazole-5-carbonitrile (35 mg, 0.09 mmol, Example 9A, Step 5), with 1-(2-fluoroethyl)-4-(4,4,5,5-tetramethyl-1,3,2-dioxaborolan-2-yl)-1H-pyrazole (24 mg, 0.10 mmol), and sodium carbonate (29 mg, 0.28 mmol) was dissolved in MeCN (1 mL) and water (200 μL). The solution was purged with nitrogen for 2 min, followed by addition of Pd(dppf)Cl₂ CH₂Cl₂ (15 mg, 0.02 mmol). The vial was sealed and the reaction was stirred at 140° C. for 5 min before being cooled to rt, diluted with CH₂Cl₂ (5 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₂FN₁₀ (M+H)⁺: m/z=457.2; found: 457.3. ¹H NMR (500 MHz, DMSO-d₆, mixture of tautomers) δ 8.85-8.77 (m, 2H), 8.74-8.68 (m, 1.45H), 8.63-8.58 (m, 1.1H), 8.49 (s, 0.45H), 8.39 (s, 0.55H), 8.35-8.28 (m, 1H), 8.19 (s, 0.45H), 4.91-4.67 (m, 3H), 4.65-4.52 (m, 2H), 4.24-4.07 (m, 1H), 2.48-2.41 (m, 0.55H), 2.39-2.31 (m, 0.45H), 2.14-1.89 (m, 5H), 1.75-1.53 (m, 1H), 1.53-1.41 (m, 1H).

### Example 71. 3-((1S,3R)-3-((5-Cyano-4-(1-cyclopropyl-1H-pyrazol-4-yl)pyrimidin-2-yl)amino)cyclohexyl)-3H-imidazo[4,5-b]pyridine-6-carbonitrile

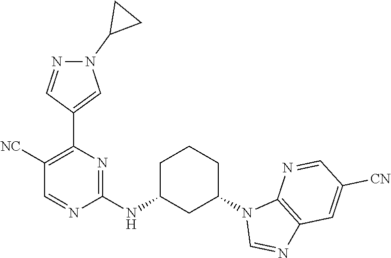 [SMILES: N#Cc1cnc2c(c1)ncn2[C@H]1CCC[C@@H](Nc2ncc(C#N)c(-c3cnn(C4CC4)c3)n2)C1]

This compound was prepared according to the procedures described in Example 9A, with 1-cyclopropyl-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₄H₂₃N₁₀ (M+H)⁺: m/z=451.2; found: 451.3. ¹H NMR (600 MHz, DMSO-d₆, mixture of tautomers) δ 8.83 (s, 1H), 8.82 (dd, J=9.1, 2.1 Hz, 1H), 8.71 (s, 1H), 8.70 (s, 0.4H), 8.61 (s, 0.6H), 8.57 (s, 0.6H), 8.46 (s, 0.4H), 8.32 (dd, J=7.8, 4.4 Hz, 1H), 8.29 (s, 0.6H), 8.09 (s, 0.4H), 4.88-4.68 (m, 1H), 4.23-4.07 (m, 1H), 3.97-3.90 (m, 1H), 2.44 (d, J=11.6 Hz, 0.6H), 2.33 (d, J=11.8 Hz, 0.4H), 2.13-1.91 (m, 5H), 1.75-1.63 (m, 0.6H), 1.63-1.54 (m, 0.4H), 1.52-1.42 (m, 1H), 1.17-1.08 (m, 2H), 1.05 (ddt, J=19.3, 7.3, 3.5 Hz, 2H).

### Example 72. 3-((1S,3R)-3-((5-Cyano-4-(1-isopropyl-1H-pyrazol-4-yl)pyrimidin-2-yl)amino)cyclohexyl)-3H-imidazo[4,5-b]pyridine-6-carbonitrile

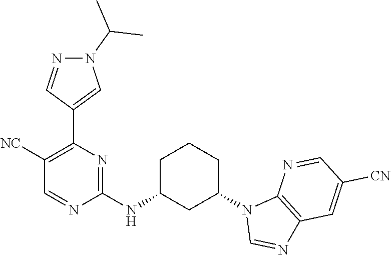 [SMILES: CC(C)n1cc(-c2nc(N[C@@H]3CCC[C@H](n4cnc5cc(C#N)cnc54)C3)ncc2C#N)cn1]

This compound was prepare according to the procedures described in Example 9A, with 1-isopropyl-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₄H₂₅N₁₀ (M+H)⁺: m/z=453.2; found: 453.3.

### Example 73. 3-((1S,3R)-3-((5-Cyano-4-(1-ethyl-1H-pyrazol-4-yl)pyrimidin-2-yl)amino)cyclohexyl)-3H-imidazo[4,5-b]pyridine-6-carbonitrile

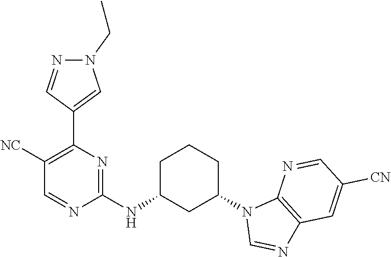 [SMILES: CCn1cc(-c2nc(N[C@@H]3CCC[C@H](n4cnc5cc(C#N)cnc54)C3)ncc2C#N)cn1]

This compound was prepared according to the procedures described in Example 9A, with 1-ethyl-4-(4,4,5,5-tetramethyl-1,3,2-dioxaborolan-2-yl)-1H-pyrazole replacing 1-(2,2-difluoroethyl)-4-(4,4,5,5-tetramethyl-1,3,2-dioxaborolan-2-yl)-1H-pyrazole in Step 6. LCMS calculated for C₂₃H₂₃N₁₀ (M+H)⁺: m/z=439.2; found: 439.3.

### Example 74. 2-(((1R,3S)-3-(6-(4-Acetylpiperazin-1-yl)-3H-imidazo[4,5-b]pyridin-3-yl)cyclohexyl)amino)-4-(1-(2,2-difluoroethyl)-1H-pyrazol-4-yl)pyrimidine-5-carbonitrile

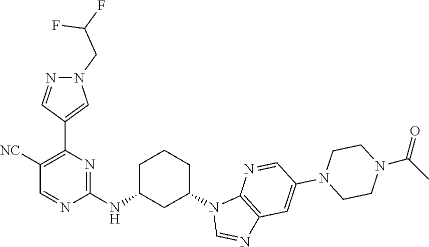 [SMILES: CC(=O)N1CCN(c2cnc3c(c2)ncn3[C@H]2CCC[C@@H](Nc3ncc(C#N)c(-c4cnn(CC(F)F)c4)n3)C2)CC1]

### Step 1. tert-Butyl ((1R,3S)-3-((5-bromo-3-nitropyridin-2-yl)amino)cyclohexyl)carbamate

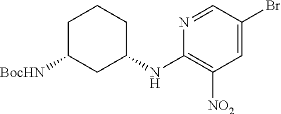 [SMILES: CC(C)(C)OC(=O)N[C@@H]1CCC[C@H](Nc2ncc(Br)cc2[N+](=O)[O-])C1]

A mixture of tert-butyl ((1R,3S)-3-aminocyclohexyl)carbamate (1.35 g, 6.3 mmol), 5-bromo-2-chloro-3-nitropyridine (1.5 g, 6.3 mmol) and DIPEA (2.2 mL, 12.6 mmol) in EtOH (30 mL) was heated to 80° C. and stirred for 2 h. Upon completion, the reaction mixture was cooled to rt and concentrated in vacuo. The crude residue was purified by Biotage Isolera (CH₂Cl₂/MeOH, up to 10% MeOH) to give the desired product as a yellow solid (2.4 g, 91% yield). LCMS calculated for C₁₂H₁₆BrN₄O₄ (M+H−C₄H₈)⁺: m/z=359.0/361.0; found: 359.1/361.1.

### Step 2. tert-Butyl ((1R,3S)-3-((3-amino-5-bromopyridin-2-yl)amino)cyclohexyl)carbamate

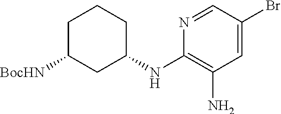 [SMILES: CC(C)(C)OC(=O)N[C@@H]1CCC[C@H](Nc2ncc(Br)cc2N)C1]

A vial containing tert-butyl ((1R,3S)-3-((5-bromo-3-nitropyridin-2-yl)amino)cyclohexyl)carbamate (1.5 g, 3.6 mmol) and iron (807 mg, 14.4 mmol) in acetic acid (20 mL) was stirred at 50° C. for 2 h. The reaction mixture was cooled to rt, diluted with MeOH and filtered through Celite. The filtrate was concentrated in vacuo, diluted with EtOAc and washed by sat. aq. NaHCO₃ solution, then brine. The organic layer was dried over MgSO₄, filtered and then concentrated in vacuo to give the desired product as a brown solid. LCMS calculated for C₁₂H₁₈BrN₄O₂ (M+H−C₄H₈)⁺: m/z=329.1; found: 329.1.

### Step 3. tert-Butyl ((1R,3S)-3-(6-bromo-3H-imidazo[4,5-b]pyridin-3-yl)cyclohexyl)carbamate

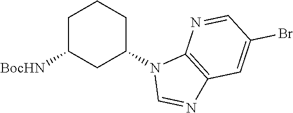 [SMILES: CC(C)(C)OC(=O)N[C@@H]1CCC[C@H](n2cnc3cc(Br)cnc32)C1]

A vial containing tert-butyl ((1R,3S)-3-((3-amino-5-bromopyridin-2-yl)amino)cyclohexyl)carbamate (1.5 g, 3.9 mmol), triethyl orthoformate (3.1 mL, 19.47 mmol), and p-toluenesulfonic acid monohydrate (10 mg, 0.05 mmol) as a solution in toluene (15 mL) was sealed and heated to 90° C. for 2 h. Upon completion, the reaction mixture was concentrated in vacuo. The resulting residue was purified by Biotage Isolera (CH₂Cl₂/MeOH, up to 10% MeOH) to give the desired product as a colorless solid (1.2 g, 78% yield). LCMS calculated for C₁₇H₂₄BrN₄O₂(M+H)⁺: m/z=395.1/397.1; found: 395.1/397.1.

### Step 4. (1R,3S)-3-(6-Bromo-3H-imidazo[4,5-b]pyridin-3-yl)cyclohexan-1-amine

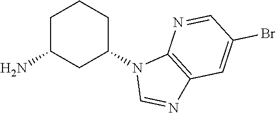 [SMILES: N[C@@H]1CCC[C@H](n2cnc3cc(Br)cnc32)C1]

To a solution of tert-butyl ((1R,3S)-3-(6-bromo-3H-imidazo[4,5-b]pyridin-3-yl)cyclohexyl)carbamate (1.1 g, 3.2 mmol) in MeOH (3 mL) was added 4N HCl/dioxane (5 mL) and left to stir at rt for 1 h. Upon completion, the reaction mixture was concentrated in vacuo. The obtained crude product was used in the next step without further purification. LCMS calculated for C₁₂H₁₆BrN₄ (M+H)⁺: m/z=295.1/297.1; found: 295.2/297.2.

### Step 5. 2-(((1R,3S)-3-(6-Bromo-3H-imidazo[4,5-b]pyridin-3-yl)cyclohexyl)amino)-4-(1-(2,2-difluoroethyl)-1H-pyrazol-4-yl)pyrimidine-5-carbonitrile

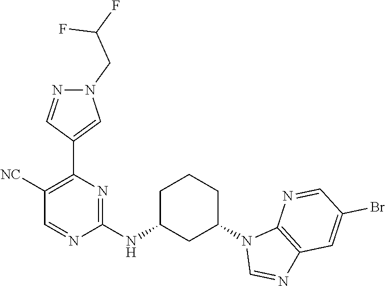 [SMILES: N#Cc1cnc(N[C@@H]2CCC[C@H](n3cnc4cc(Br)cnc43)C2)nc1-c1cnn(CC(F)F)c1]

A vial containing a mixture of (1R,3S)-3-(6-bromo-3H-imidazo[4,5-b]pyridin-3-yl)cyclohexan-1-amine (457 mg, 1.5 mmol), 4-(1-(2,2-difluoroethyl)-1H-pyrazol-4-yl)-2-(methylsulfonyl)pyrimidine-5-carbonitrile (Intermediate 1, 440 mg, 1.4 mmol) and DIPEA (735 μL, 4.2 mmol) in EtOH (10 mL) was heated to 60° C. for 1 h. The reaction mixture was concentrated in vacuo and the resulting residue was purified by Biotage Isolera (CH₂Cl₂/MeOH, up to 10% MeOH) to give the product as a light yellow solid (700 mg, 87% yield). LCMS calculated for C₂₂H₂₁BrF₂N₉ (M+H)⁺: m/z=528.1; found: 528.2.

### Step 6. 2-(((1R,3S)-3-(6-(4-Acetylpiperazin-1-yl)-3H-imidazo[4,5-b]pyridin-3-yl)cyclohexyl)amino)-4-(1-(2,2-difluoroethyl)-1H-pyrazol-4-yl)pyrimidine-5-carbonitrile

A vial containing 2-(((1R,3S)-3-(6-bromo-3H-imidazo[4,5-b]pyridin-3-yl)cyclohexyl)amino)-4-(1-(2,2-difluoroethyl)-1H-pyrazol-4-yl)pyrimidine-5-carbonitrile (30 mg, 0.06 mmol), 1-(piperazin-1-yl)ethan-1-one (7.3 mg, 0.06 mmol), cesium carbonate (55 mg, 0.17 mmol), and RuPhos Pd G4 (9.7 mg, 0.01 mmol) was evacuated and backfilled with nitrogen three times, followed by the addition of 1,4-dioxane (1 mL). The vial was sealed and heated to 110° C. for 16 h. After cooling to rt, the mixture was filtered through a Silicycle SiliaPrep™ Thiol cartridge (Cat. #SPE-R51030B), washed with MeCN, and purified with prep-LCMS (XBridge C₁₈ column, eluting with a gradient of acetonitrile/water containing 0.1% TFA, at flow rate of 60 mL/min). LCMS calculated for C₂₈H₃₂F₂N₁₁O (M+H)⁺: m/z=576.3; found: 576.2.

### Example 75. 4-(1-(2,2-Difluoroethyl)-1H-pyrazol-4-yl)-2-(((1R,3S)-3-(6-(4-methylpiperazin-1-yl)-3H-imidazo[4,5-b]pyridin-3-yl)cyclohexyl)amino)pyrimidine-5-carbonitrile

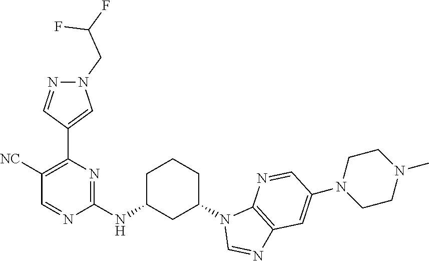 [SMILES: CN1CCN(c2cnc3c(c2)ncn3[C@H]2CCC[C@@H](Nc3ncc(C#N)c(-c4cnn(CC(F)F)c4)n3)C2)CC1]

This compound was prepared according to the procedures described in Example 74, with 1-methylpiperazine replacing 1-(piperazin-1-yl)ethan-1-one in Step 6. LCMS calculated for C₂₇H₃₂F₂Nn (M+H)⁺: m/z=548.3; found: 548.3.

### Example 76. 4-(1-(2,2-Difluoroethyl)-1H-pyrazol-4-yl)-2-(((1R,3S)-3-(6-morpholino-3H-imidazo[4,5-b]pyridin-3-yl)cyclohexyl)amino)pyrimidine-5-carbonitrile

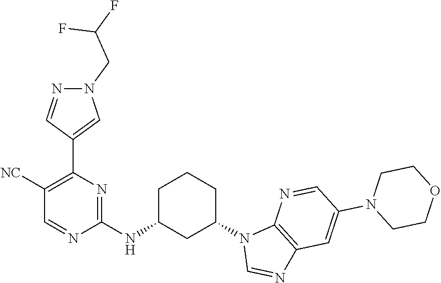 [SMILES: N#Cc1cnc(N[C@@H]2CCC[C@H](n3cnc4cc(N5CCOCC5)cnc43)C2)nc1-c1cnn(CC(F)F)c1]

This compound was prepared according to the procedures described in Example 74, with morpholine replacing 1-(piperazin-1-yl)ethan-1-one in Step 6. LCMS calculated for C₂₆H₂₉F₂N₁₀O (M+H)⁺: m/z=535.2; found: 535.3.

### Example 77. 4-(1-(2,2-Difluoroethyl)-1H-pyrazol-4-yl)-2-(((1R,3S)-3-(6-(4-methylpyridin-3-yl)-3H-imidazo [4,5-b]pyridin-3-yl)cyclohexyl)amino)pyrimidine-5-carbonitrile

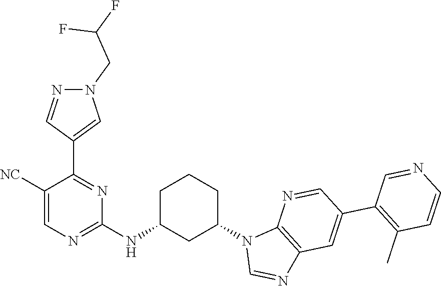 [SMILES: Cc1ccncc1-c1cnc2c(c1)ncn2[C@H]1CCC[C@@H](Nc2ncc(C#N)c(-c3cnn(CC(F)F)c3)n2)C1]

A vial containing 2-(((1R,3S)-3-(6-bromo-3H-imidazo[4,5-b]pyridin-3-yl)cyclohexyl)amino)-4-(1-(2,2-difluoroethyl)-1H-pyrazol-4-yl)pyrimidine-5-carbonitrile (30 mg, 0.06 mmol, Example 74, Step 5), 4-methyl-3-(4,4,5,5-tetramethyl-1,3,2-dioxaborolan-2-yl)pyridine (7 mg, 0.06 mmol), cesium carbonate (55 mg, 0.17 mmol), and XPhos Pd G2 (10 mg, 0.011 mmol) was evacuated and backfilled with nitrogen three times, followed by the addition of 1,4-dioxane (1 mL) and water (200 μL). The vial was sealed and heated to 80° C. for 2 h. After cooling to rt, the mixture was diluted with EtOAc, washed with water and brine, and the organic layer was dried over MgSO₄, filtered and concentrated in vacuo. The residue was diluted with MeOH and filtered through a Silicycle SiliaPrep™ Thiol cartridge (Cat. #SPE-R51030B) and purified with prep-LCMS (XBridge C₁₈ column, eluting with a gradient of acetonitrile/water containing 0.1% TFA, at flow rate of 60 mL/min). LCMS calculated for C₂₈H₂₇F₂N₁₀ (M+H)⁺: m/z=541.2; found: 541.2.

### Example 78. 4-(1-(2,2-Difluoroethyl)-1H-pyrazol-4-yl)-N-((1R,3S)-3-(6-(prop-1-en-2-yl)-3H-imidazo[4,5-b]pyridin-3-yl)cyclohexyl)-5-(trifluoromethyl)pyrimidin-2-amine

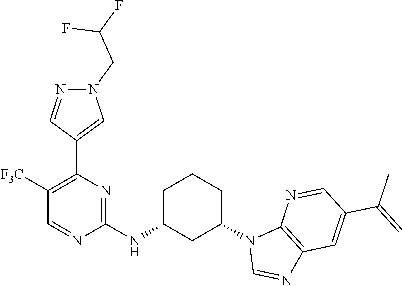 [SMILES: C=C(C)c1cnc2c(c1)ncn2[C@H]1CCC[C@@H](Nc2ncc(C(F)(F)F)c(-c3cnn(CC(F)F)c3)n2)C1]

### Step 1. N—((1R,3S)-3-(6-Bromo-3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine

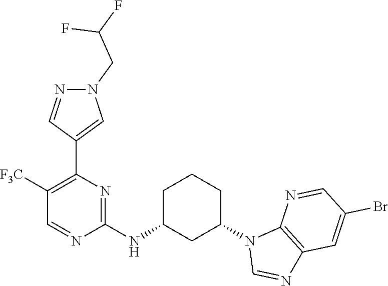 [SMILES: FC(F)Cn1cc(-c2nc(N[C@@H]3CCC[C@H](n4cnc5cc(Br)cnc54)C3)ncc2C(F)(F)F)cn1]

A vial containing a solution of (1R,3S)-3-(6-bromo-3H-imidazo[4,5-b]pyridin-3-yl)cyclohexan-1-amine (457 mg, 1.5 mmol, Example 74, Step 4), 2-chloro-4-(1-(2,2-difluoroethyl)-1H-pyrazol-4-yl)-5-(trifluoromethyl)pyrimidine (440 mg, 1.4 mmol, Example 15, Step 2) and DIPEA (735 μL, 4.22 mmol) in EtOH (10 mL) was heated at 60° C. for 1 h. The reaction mixture was concentrated in vacuo and the crude residue was purified by Biotage Isolera (CH₂Cl₂/MeOH, up to 10% MeOH) to give the product as a light yellow solid (720 mg, 90% yield). LCMS calculated for C₂₂H₂₁BrF₅N₈(M+H)⁺: m/z=571.1; found: 571.2.

### Step 2. 4-(1-(2,2-Difluoroethyl)-1H-pyrazol-4-yl)-N-((R,3S)-3-(6-(prop-1-en-2-yl)-3H-imidazo[4,5-b]pyridin-3-yl)cyclohexyl)-5-(trifluoromethyl)pyrimidin-2-amine

A vial containing N-((1R,3S)-3-(6-bromo-3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine (55 mg, 0.1 mmol), 4,4,5,5-tetramethyl-2-(prop-1-en-2-yl)-1,3,2-dioxaborolane (21 μL, 0.12 mmol), XPhos Pd G2 (9.7 mg, 0.01 mmol), tripotassium phosphate (50 mg, 0.24 mmol) was evacuated and backfilled with nitrogen three times, followed by the addition of 1,4-dioxane (1 mL) and water (200 μL). The vial was sealed and heated to 80° C. for 2 h. After cooling to rt, the mixture was diluted with EtOAc, washed with water and brine, and the organic layer was dried over MgSO₄, filtered and concentrated in vacuo. The residue was diluted with MeOH and filtered through a Silicycle SiliaPrep™ Thiol cartridge (Cat. #SPE-R51030B) and purified with prep-LCMS (XBridge C₁₈ column, eluting with a gradient of acetonitrile/water containing 0.1% TFA, at flow rate of 60 mL/min). LCMS calculated for C₂₅H₂₆F₅N₈ (M+H)⁺: m/z=533.2; found: 533.2.

### Example 79. N-((1R,3S)-3-(6-Cyclopropyl-3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine

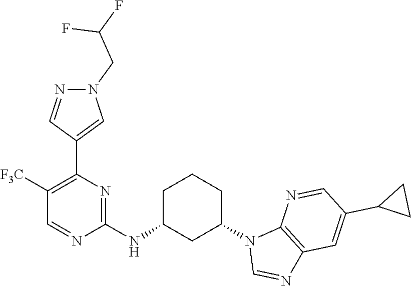 [SMILES: FC(F)Cn1cc(-c2nc(N[C@@H]3CCC[C@H](n4cnc5cc(C6CC6)cnc54)C3)ncc2C(F)(F)F)cn1]

This compound was prepared according to the procedures described in Example 78, with potassium cyclopropyltrifluoroborate replacing 4,4,5,5-tetramethyl-2-(prop-1-en-2-yl)-1,3,2-dioxaborolane in Step 2. LCMS calculated for C₂₅H₂₆F₅N₈ (M+H)⁺: m/z=533.2; found: 533.3.

### Example 80. N-((1R,3S)-3-(6-(1H-Pyrazol-4-yl)-3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine

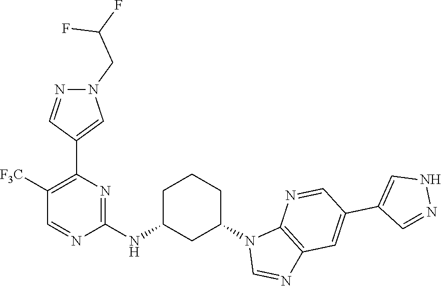 [SMILES: FC(F)Cn1cc(-c2nc(N[C@@H]3CCC[C@H](n4cnc5cc(-c6cn[nH]c6)cnc54)C3)ncc2C(F)(F)F)cn1]

This compound was prepared according to the procedures described in Example 78, with 4-(4,4,5,5-tetramethyl-1,3,2-dioxaborolan-2-yl)-1H-pyrazole replacing 4,4,5,5-tetramethyl-2-(prop-1-en-2-yl)-1,3,2-dioxaborolane in Step 2. LCMS calculated for C₂₅H₂₄F₅N₁₀ (M+H)⁺: m/z=559.2; found: 559.3.

### Example 81. 4-(1-(2,2-Difluoroethyl)-1H-pyrazol-4-yl)-N-((1R,3S)-3-(6-(1-methyl-1H-pyrazol-4-yl)-3H-imidazo[4,5-b]pyridin-3-yl)cyclohexyl)-5-(trifluoromethyl)pyrimidin-2-amine

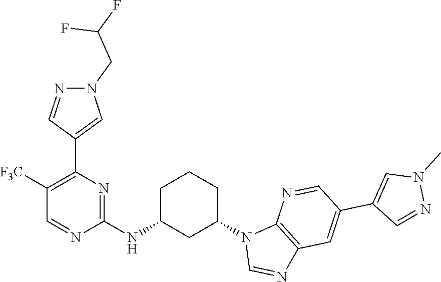 [SMILES: Cn1cc(-c2cnc3c(c2)ncn3[C@H]2CCC[C@@H](Nc3ncc(C(F)(F)F)c(-c4cnn(CC(F)F)c4)n3)C2)cn1]

This compound was prepared according to the procedures described in Example 78, with 1-methyl-4-(4,4,5,5-tetramethyl-1,3,2-dioxaborolan-2-yl)-1H-pyrazole replacing 4,4,5,5-tetramethyl-2-(prop-1-en-2-yl)-1,3,2-dioxaborolane in Step 2. LCMS calculated for C₂₆H₂₆F₅N₁₀ (M+H)⁺: m/z=573.2; found: 573.3.

### Example 82. 4-(1-(2,2-Difluoroethyl)-1H-pyrazol-4-yl)-N-((1R,3S)-3-(6-(pyridin-3-yl)-3H-imidazo[4,5-b]pyridin-3-yl)cyclohexyl)-5-(trifluoromethyl)pyrimidin-2-amine

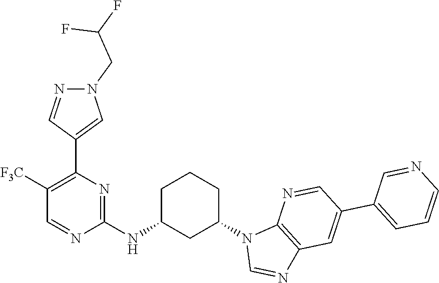 [SMILES: FC(F)Cn1cc(-c2nc(N[C@@H]3CCC[C@H](n4cnc5cc(-c6cccnc6)cnc54)C3)ncc2C(F)(F)F)cn1]

This compound was prepared according to the procedures described in Example 78, with 3-(4,4,5,5-tetramethyl-1,3,2-dioxaborolan-2-yl)pyridine replacing 4,4,5,5-tetramethyl-2-(prop-1-en-2-yl)-1,3,2-dioxaborolane in Step 2. LCMS calculated for C₂₇H₂₅F₅N₉ (M+H)⁺: m/z=570.2; found: 570.3.

### Example 83. 4-(1-(2,2-Difluoroethyl)-1H-pyrazol-4-yl)-N-((1R,3S)-3-(6-(pyrrolidin-1-yl)-3H-imidazo[4,5-b]pyridin-3-yl)cyclohexyl)-5-(trifluoromethyl)pyrimidin-2-amine

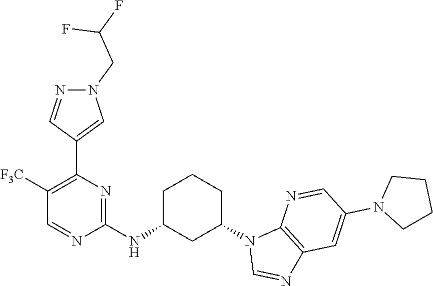 [SMILES: FC(F)Cn1cc(-c2nc(N[C@@H]3CCC[C@H](n4cnc5cc(N6CCCC6)cnc54)C3)ncc2C(F)(F)F)cn1]

A vial containing N-((1R,3S)-3-(6-bromo-3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine (30 mg, 0.05 mmol, Example 78, Step 1), pyrrolidine (8.8 μL, 0.1 mmol), cesium carbonate (51 mg, 0.16 mmol), and RuPhos Pd G4 (9 mg, 0.01 mmol) was evacuated and backfilled with nitrogen three times, followed by the addition of 1,4-dioxane (1 mL). The vial was sealed and heated to 110° C. for 16 h. After cooling to rt, the reaction mixture was filtered through a Silicycle SiliaPrep™ Thiol cartridge (Cat. #SPE-R51030B) with MeCN and purified with prep-LCMS (XBridge C₁₈ column, eluting with a gradient of acetonitrile/water containing 0.1% TFA, at flow rate of 60 mL/min). LCMS calculated for C₂₆H₂₉F₅N₉ (M+H)⁺: m/z=562.2; found: 562.2.

### Example 84. 3-((1S,3R)-3-((4-(1-(2,2-Difluoroethyl)-1H-pyrazol-4-yl)-5-(trifluoromethyl)pyrimidin-2-yl)amino)cyclohexyl)-N,N-dimethyl-3H-imidazo[4,5-b]pyridin-6-amine

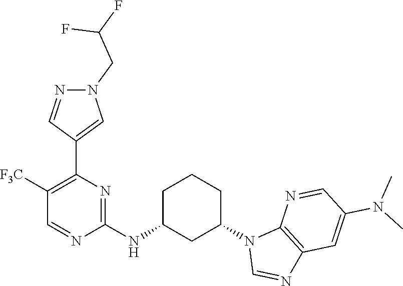 [SMILES: CN(C)c1cnc2c(c1)ncn2[C@H]1CCC[C@@H](Nc2ncc(C(F)(F)F)c(-c3cnn(CC(F)F)c3)n2)C1]

This compound was prepared according to the procedures described in Example 83, with dimethylamine replacing pyrrolidine. LCMS calculated for C₂₄H₂₇F₅N₉ (M+H)⁺: m/z=536.2; found: 536.3.

### Example 85. 4-(1-(2,2-Difluoroethyl)-1H-pyrazol-4-yl)-2-(((1R,3S)-3-(4-methyl-1H-imidazo[4,5-c]pyridin-1-yl)cyclohexyl)amino)pyrimidine-5-carbonitrile

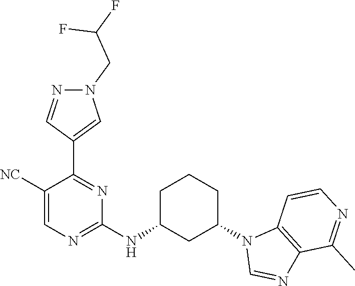 [SMILES: Cc1nccc2c1ncn2[C@H]1CCC[C@@H](Nc2ncc(C#N)c(-c3cnn(CC(F)F)c3)n2)C1]

This compound was prepared according to the procedures described in Example 9A, with 4-chloro-2-methyl-3-nitropyridine replacing 6-chloro-5-nitronicotinonitrile in Step 1. LCMS calculated for C₂₃H₂₄F₂N₉ (M+H)⁺: m/z=464.2; found: 464.2.

### Example 86. 1-((1S,3R)-3-((5-Cyano-4-(1-(2,2-difluoroethyl)-1H-pyrazol-4-yl)pyrimidin-2-yl)amino)cyclohexyl)-1H-benzo[d]imidazole-5-carbonitrile

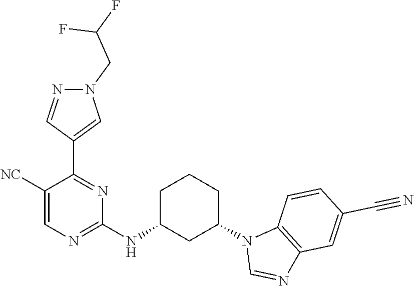 [SMILES: N#Cc1ccc2c(c1)ncn2[C@H]1CCC[C@@H](Nc2ncc(C#N)c(-c3cnn(CC(F)F)c3)n2)C1]

This compound was prepared according to the procedures described in Example 9A, with 4-fluoro-3-nitrobenzonitrile replacing 6-chloro-5-nitronicotinonitrile in Step 1. LCMS calculated for C₂₄H₂₂F₂N₉ (M+H)⁺: m/z=474.2; found: 474.2.

### Example 87. 1-((1S,3R)-3-((4-(1-(2,2-Difluoroethyl)-1H-pyrazol-4-yl)-5-(trifluoromethyl)pyrimidin-2-yl)amino)cyclohexyl)-1H-benzo[d]imidazole-7-carbonitrile

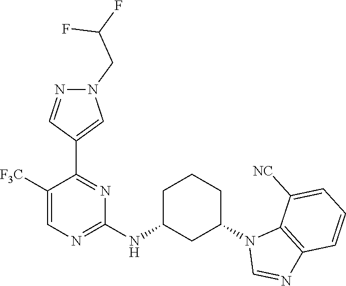 [SMILES: N#Cc1cccc2ncn([C@H]3CCC[C@@H](Nc4ncc(C(F)(F)F)c(-c5cnn(CC(F)F)c5)n4)C3)c12]

This compound was prepared according to the procedures described in Example 1, with 2-fluoro-3-nitrobenzonitrile replacing 6-chloro-5-nitronicotinonitrile in Step 1. LCMS calculated for C₂₄H₂₂F₅N₈ (M+H)⁺: m/z=517.2; found: 517.3. ¹H NMR (500 MHz, DMSO-d₆, mixture of tautomers) δ 8.85 (s, 0.55H), 8.81 (s, 0.45H), 8.62 (s, 0.45H), 8.55 (s, 0.55H), 8.36 (s, 0.55H), 8.28 (s, 0.45H), 8.17-8.05 (m, 2.55H), 7.99 (s, 0.45H), 7.86 (d, J=7.5 Hz, 1H), 7.45 (t, J=7.9 Hz, 1H), 6.56-6.23 (m, 1H), 5.04-4.83 (m, 1H), 4.82-4.70 (m, 2H), 4.24-4.06 (m, 1H), 2.54 (s, 1H), 2.30-2.18 (m, 1H), 2.13-1.82 (m, 4H), 1.70-1.52 (m, 1H), 1.52-1.38 (m, 1H).

### Example 88. 3-((1S,3R)-3-((5-Cyano-4-(1-(2,2-difluoroethyl)-1H-pyrazol-4-yl)pyrimidin-2-yl)amino)cyclohexyl)-3H-imidazo[4,5-c]pyridine-6-carbonitrile

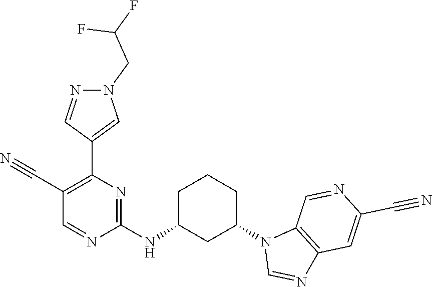 [SMILES: N#Cc1cc2ncn([C@H]3CCC[C@@H](Nc4ncc(C#N)c(-c5cnn(CC(F)F)c5)n4)C3)c2cn1]

This compound was prepared according to the procedures described in Example 46, with 2-bromo-5-fluoro-4-nitropyridine replacing 2-bromo-4-chloro-5-nitropyridine in Step 1. LCMS calculated for C₂₃H₂₁F₂N₁₀ (M+H)⁺: m/z=475.2; found: 475.2.

### Example 89. 3-((1S,3R)-3-((5-Cyano-4-(1,5-dimethyl-1H-pyrazol-4-yl)pyrimidin-2-yl)amino)cyclohexyl)-3H-imidazo[4,5-b]pyridine-6-carbonitrile

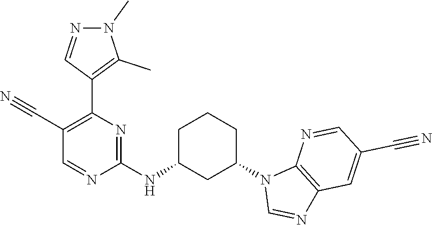 [SMILES: Cc1c(-c2nc(N[C@@H]3CCC[C@H](n4cnc5cc(C#N)cnc54)C3)ncc2C#N)cnn1C]

A mixture of 1-((1S,3R)-3-((4-chloro-5-cyanopyrimidin-2-yl)amino)cyclohexyl)-1H-benzo[d]imidazole-5-carbonitrile (10 mg, 0.026 mmol, Example 9A, Step 5), 1,5-dimethyl-4-(4,4,5,5-tetramethyl-1,3,2-dioxaborolan-2-yl)-1H-pyrazole (9 mg, 0.04 mmol), and sodium carbonate (8 mg, 0.08 mmol) was dissolved in MeCN (1 mL) and water (200 μL). The solution was purged with nitrogen for 2 min, followed by addition of Pd(dppf)Cl₂ CH₂Cl₂ (2 mg, 2.6 μmol). The vial was sealed and the reaction was stirred at 140° C. for 5 min before being cooled to rt, diluted with CH₂Cl₂ (5 mL), and filtered through a plug of Celite. The filtrate was concentrated in vacuo. The resulting residue was diluted with MeCN, water, and TFA and purified by prep-LCMS (XBridge C₁₈ column, eluting with a gradient of acetonitrile/water containing 0.1% TFA, at flow rate of 60 mL/min). LCMS calculated for C₂₃H₂₃N₁₀ (M+H)⁺: m/z=439.2; found: 439.2.

### Example A1. CDK Enzymatic Assays

The following activity of the CDK enzymes in complex with their respective cyclins we assayed: CDK1 complexed with Cyclin B1; CDK2 complexed with Cyclin E1; CDK4 complexed with Cyclin D1; CDK6 complexed with Cyclin D1; CDK6 complexed with Cyclin D3; CDK9 complexed with Cyclin T1; CDK7 complexed with Cyclin H/MAT1; CDK2 complexed with Cyclin A2; CDK5 complexed with p35; CDK12 complexed with Cyclin K; CDK13 complexed with Cyclin K. These in vitro enzyme activity are assayed using homogeneous time-resolved energy transfer (HTRF), which measures phosphorylation of a peptide substrate. The LANCE® Ultra kinase assay (PerkinElmer) uses a ULight™-labeled EIF4E-binding protein 1 (THR37/46) peptide (DYSTTPGGTLFSTTPGTRI) (SEQ ID NO: 1) substrate and a Europium-labeled anti-phospho-4E-BP1 antibody (CDK2, CDK1, CDK4, CDK6, CDK9, CDK12, CDK13) or ULight™-labeled Myelin Basic Protein peptide (VTPRTPPP) (SEQ ID NO: 2) substrate and a Europium-labeled anti-phospho-MBP antibody (CDK7). Each CDK enzyme activity assays utilized human CDK co-expressed as N-terminal GST-tagged protein with its full length cyclin partner using a baculovirus expression system.

Enzyme was pre-incubated with compounds for 30 minutes (CDK1,2,4,6,9) or 60 minutes (CDK7, CDK12, CDK13) prior to addition of ATP and Ulight-peptide (1 mM and 50 nM final, respectively), in assay buffer containing 50 mM HEPES pH 7.5, 1 mM EGTA, 10 mM MgCl₂, 2 mM DTT, 0.05 mg/mL BSA, and 0.01% Tween 20.

The reaction was then incubated for 60-90 minutes at room temperature. The reactions were stopped by the addition of EDTA and Europium labeled antibody, for a final concentration of 15 mM and 1.0-1.5 nM, respectively. HTRF signals were read after 15-120 minutes. A ratio of fluorescence transferred to the labeled substrate (665 nm) relative to fluorescence of the Europium donor (620 nm) represents the extent of phosphorylation. Ratios for treated wells were normalized to DMSO only (100% activity) and no enzyme (0% activity) controls. Normalized data was analyzed using a three or four parameter dose response curve to determine IC₅₀ for each compound and are shown in Table A. Control reference inhibitors were included on each plate.

TABLE A

| Ex. | CDK12 |
|---|---|
| No. | IC₅₀ (nM) |
| 1 | + |
| 2 | +++ |
| 3 | + |
| 4 | + |
| 5 | + |
| 6 | + |
| 7 | + |
| 8 | + |
| 9/9A | + |
| 10 | + |
| 11 | + |
| 12 | + |
| 13/13A | + |
| 14 | + |
| 15 | + |
| 16 | ++ |
| 17 | ++ |
| 18 | + |
| 19 | +++ |
| 20 | ++ |
| 21 | + |
| 22 | +++ |
| 23 | ++ |
| 24 | + |
| 25 | ++ |
| 26 | ++ |
| 27 | + |
| 28/28A | + |
| 29 | ++ |
| 30 | ++ |
| 31 | + |
| 32 | + |
| 33 | + |
| 34 | + |
| 35 | +++ |
| 36 | + |
| 37 | + |
| 38 | + |
| 39 | + |
| 40 | + |
| 41 | + |
| 42 | + |
| 43 | + |
| 45 | + |
| 46 | + |
| 47 | + |
| 48 | + |
| 49 | + |
| 50 | + |
| 51 | ++ |
| 52 | + |
| 53 | + |
| 54 | + |
| 55 | + |
| 56 | +++ |
| 57 | ++ |
| 58 | + |
| 59 | + |
| 60 | ++ |
| 61 | + |
| 62 | + |
| 63 | + |
| 64 | + |
| 65 | + |
| 66 | + |
| 67 | + |
| 68 | + |
| 69 | + |
| 70 | + |
| 71 | + |
| 72 | + |
| 73 | + |
| 74 | + |
| 75 | + |
| 76 | + |
| 77 | ++ |
| 78 | + |
| 79 | + |
| 80 | + |
| 81 | + |
| 82 | + |
| 83 | + |
| 84 | + |
| 85 | + |
| 86 | + |
| 87 | + |
| 88 | + |
| 89 | + |

+ refers to IC₅₀ of <50 nM
++ refers to IC₅₀ of ≥50 nM to ≤500 nM
+++ refers to >500 nM

### Example A2. Alternative CDK Enzymatic Assays

The following activity of the CDK enzymes in complex with their respective cyclins we assayed: CDK1 complexed with Cyclin B1; CDK2 complexed with Cyclin E1; CDK4 complexed with Cyclin D1; CDK6 complexed with Cyclin D1; CDK6 complexed with Cyclin D3; CDK9 complexed with Cyclin T1; CDK7 complexed with Cyclin H/MAT1; CDK2 complexed with Cyclin A2; CDK5 complexed with p35; CDK12 complexed with Cyclin K; CDK13 complexed with Cyclin K. These in vitro enzyme activity are assayed using homogeneous time-resolved energy transfer (HTRF), which measures phosphorylation of a peptide substrate. The LANCE® Ultra kinase assay (PerkinElmer) uses a ULight™-labeled EIF4E-binding protein 1 (THR37/46) peptide (DYSTTPGGTLFSTTPGTRI) (SEQ ID NO: 1) substrate and a Europium-labeled anti-phospho-4E-BP1 antibody (CDK2, CDK1, CDK4, CDK6, CDK9, CDK12, CDK13) or ULight™-labeled Myelin Basic Protein peptide (VTPRTPPP) (SEQ ID NO: 2) substrate and a Europium-labeled anti-phospho-MBP antibody (CDK7). Each CDK enzyme activity assays utilized human CDK co-expressed as N-terminal GST-tagged protein with its full length cyclin partner using a baculovirus expression system.

Enzyme was pre-incubated with compounds and 1 mM ATP for 60 minutes (CDK 1, 2, 4, 6, 9, 7, 12, 13) prior to the addition of ATP and Ulight-peptide (1 mM and 50 nM final, respectively), in assay buffer containing 50 mM HEPES pH 7.5, 1 mM EGTA, 10 mM MgCl₂, 2 mM DTT, 0.05 mg/mL BSA, and 0.01% Tween 20. The reaction was then incubated for 120 minutes at 25° C. incubator. The reactions were stopped by the addition of EDTA and Europium labeled antibody, for a final concentration of 15 mM and 0.5-1.5 nM, respectively. HTRF signals were read after 15-120 minutes. A ratio of fluorescence transferred to the labeled substrate (665 nm) relative to fluorescence of the Europium donor (620 nm) represents the extent of phosphorylation. Ratios for treated wells were normalized to DMSO only (100% activity) and no enzyme (0% activity) controls. Normalized data was analyzed using a three or four parameter dose response curve to determine IC₅₀ for each compound. Control reference inhibitors were included on each plate.

### Example B. CDK Cellular Activity Assays

### A. HTRF Assay

The following signals were detected using an HTRF assay from Cisbio: CDK12/13 activity (RNA POL II pser2 HTRF assay in multiple cell lines); CDK2 activity (pRbS780 HTRF assay in COV318 cells); CDK4 (pRbS780 HTRF assay in JEKO-1 cells); CDK6 activity (pRbS780 HTRF assay in MV4-11 cells); CDK12 specific activity (RNA POL II pser2 in CDK13^{-/-} isogenic THP1 cells); CDK13 specific activity (RNA POL II pser2 in CDK12^{-/-} isogenic THP1 cells); Gamma H2AX for DNA damage (HTRF assay in multiple cell lines).

All HTRF assays were performed following the following standard protocol. First, cells were plated in a 96 well plate and treated with 3 fold dilution series of compound for 6 hours (CDK2, CDK4, CDK6, CDK12, CDK13) or 48 hours (Gamma H2AX). Then, 4× Cisbio lysis buffer was diluted 4 fold with distilled water supplemented with 100× blocking buffer and a 1:10,000 dilution of Benzonase Nuclease (Sigma Cat #E1014-5KU). Next, 50 μL of the prepared 1× Cisbio lysis buffer was added to each well of cells. The plates were gently shaken at room temperature for 30-45 minutes to lyse. The lysates were then used immediately or stored at −80° C. and processed at a later date. To process, the 96 well plates were centrifuged at 1400 rpm for 5 minutes at 4° C. Then, acceptor D2 and donor K antibody mixes were made up as follows: 50 μL of antibody+950 μL detection buffer per one 384 plate (equal to 4×96 well plates). 2 μL acceptor D2 and 2 μL of donor K antibody mixes were added to enough wells of a 384 well Greiner white plate (Greiner cat #784075) to accommodate the number of cell samples from the 96 well plate. Lastly, 16 μL of each cell lysate from the 96 well plate was transferred to the wells in the 384 well plate containing the 4 μL of acceptor D2+donor K antibody mixes (final volume of 20 μL per well). The 384 well plate was then incubated overnight at room temperature covered in foil. HTRF signal was measured on the Pherastar microplate reader the next morning.

### B. In Cell Western Blotting Assay

The following signals were detected using an in cell western blotting assay: CDK1 activity (pNPM-T199 signal); CDK7 activity (RNA POL II pser5 signal). Cells were plated at 25,000 cells per well in a 96 well plate at 37° C. and allowed to attach overnight. The next day, cells were treated with a 3 fold dilution series of compound for 6 hours. Next, media was removed and the cells were washed once with 140 μL/well of 1×PBS. The cells were then fixed with freshly diluted 3.7% paraformaldehyde/PBS for 20 minutes at room temperature. The fixing solution was removed and the cells were washed 3 times with 1×PBS containing 0.1% TX-100 for 5-10 minutes per wash with gentle shaking for permeabilization. Next, the plates were blocked by adding 50 μL/well of Odyssey blocking buffer with 0.1% TX-100 followed by rocking gently for 1 hour at room temperature. The blocking buffer was then removed and replaced with 40 μL/well of primary antibody diluted in Odyssey blocking buffer (1:200-1:500) with 0.1% TX-100 and the plates were incubated overnight with moderate shaking at 4° C. Next, the primary antibody was removed and the plates were washed 3 times with 140 μL of 1×PBS containing 0.1% Tween-20 for 10 minutes per wash with gentle shaking. Then 40 μL/well of secondary antibody (IRDye® 800CW Goat anti-Rabbit, 1:2000) and CellTag 700 (1:600) in Odyssey blocking buffer with 0.1% TX-100 was added and the plates were covered in foil and rocked gently for 2 hours at room temperature. Next, secondary antibody was removed and washed 3 times with 140 L of 1×PBS containing 0.1% Tween-20 for 10 minutes per wash with gentle shaking. The plates were protected from light during washing. After the final wash, the washing solution was completely removed from wells and the bottom plate surface and the scanning bed were cleaned with lint-free paper. The plate was scanned with detection in both 700 and 800 nm channels using an Odyssey CLx. (scanning parameters: Odyssey CLx 169 μm resolution at 3.5 mm focus offset).

### C. Standard Western Blotting

The following signal was detected using standard western blotting: CDK5 activity (pFAKT732); CDK9 activity (MCL-1 protein level).

Cells were plated overnight in a 6 well plate and treated with a 3 fold dilution of compound. Cells were then washed with ice cold PBS and then lysed using the standard Cell Signaling Lysis Protocol (Cat #9803). Cell lysates were then quantified using the standard BCA protein assay protocol from Pierce (Cat #23225) and equal amounts of protein were then run on a 4 to 12% NuPAGE gel (Cat #NP0322). Expression as analyzed following standard western blotting procedure. Briefly, membranes were blocked with 5% milk in TBST for 1 hour and then incubated with primary antibody (GET CAT #) at 1:2000 overnight. Membranes were then washed 3 times with TBST and incubated (1:4000 dilution) with secondary antibody (Cell Signaling Cat #7074). For imaging, membranes were in incubated in HRP substrate and imaged on a gel-doc imager.

### Example C. Invitrogen QuantiGene Assay

Multiplex Invitrogen QuantiGene assays are used to measure gene expression directly from cell lysates to determine expression levels following CDK12 inhibitor treatment. Target RNAs are captured through specific probe hybridization and quantified through branched DNA technology that amplifies the signal.

To prepare lysates, OVCAR3 cells are plated at a density of 20,000 cells/well in growth media (RPMI 20%+1×Pen/Strep) and placed in an incubator at 37° C. with 5% CO₂ overnight. The next day, cells are treated with CDK12 inhibitors starting at a concentration of 10 uM in a 3-fold dilution of 10 dose points for 6 hours. Following this, the cells are lysed to release the target RNA or DNA. An oligonucleotide probe set is then incubated with the target RNA or DNA with shaking at 54° C. overnight. During this incubation, the probes cooperatively hybridize to the target. On the final day of the assay, signal amplification is performed via sequential hybridization of the branched DNA pre-amplifier, amplifier, and labeled probe molecules to the target each for 1 hour with shaking at 50° C. Addition of a fluorescent reporter generates a signal directly proportional to the amount of target RNA or DNA present in the sample. The signal is read using a Luminex instrument for multiplex assays. CDK12 target genes included BRCA1, BRCA2, ERCC4, BLM. MCL-1 expression was used to assess inhibition of CDK9/7. HPRT1 expression was used for normalization.

### Example D. CDK9/12/7 Nanobret Assays

The NanoBRET Target Engagement (TE) Assay measures compound binding at select target proteins within intact cells. Compound engagement is measured in a competitive format using a cell-permeable fluorescent NanoBRET tracer. Binding of the test compound results in a loss of NanoBRET signal between the target protein and the tracer inside intact cells. First, HEK293 cells (DMEM+10% FBS) were trypsinzed and resuspended in assay media (99% Opti-MEM I Reduced Serum Medium, no phenol red (Life Technologies Cat. #11058-021) and cell density was adjusted to 2×10⁵ cells/mL. Cells were transiently transfected with Nano-Luc fusion plasmid along with their cyclin partners CDK9/CyclinT1, CDK7, CDK12/13 with Cyclin K. DNA:lipid complex was prepared using 9.0 g/ml of cyclin DNA, 1.0 g/ml of NanoLucR fusion vector DNA and in 1 mL of Opti-MEM media. Mixed thoroughly in FuGENE HD Transfection Reagent and incubated for 20 min to allow the DNA: Lipid complex formation. Mix 1 part of lipid: DNA complex (e.g., 1 mL) with 20 parts of HEK293 cells (e.g., 20 mL) in suspension at 2×10⁵ cells/mL. Mix gently by inversion 5 times. 100 μL cells+lipid: DNA complex were dispensed into a sterile tissue-culture treated 96-well assay plate and incubate 20-30 hours for cells to express the target protein. First, prepare a 100× solution of NanoBRET Tracer in 100% DMSO. For each individual target, Promega provide a recommended 100× tracer concentration as a starting point (CDK9-Tracer 8, conc. 0.063 μM; CDK7-Tracer 10, conc. 0.5 μM, CDK12-Tracer12, conc. 0.5 μM). Prepare Complete 20× NanoBRET Tracer Reagent by adding 4 parts of Tracer Dilution Buffer to 1 part of 100× NanoBRET tracer. Dispense 5 μL of Complete 20× NanoBRET Tracer Reagent per well to cells. Mix the 96-well plate on an orbital shaker for 15 seconds at 900 rpm. Prepare serially diluted test compound at 1,000× final concentration in 100% DMSO. Then dilute 1,000× test compound to 10× final concentration in Opti-MEM I Reduced Serum Medium, no phenol red. Add 10 μL of 10× serially diluted test compound per well of 96-well plates containing cells with 1× NanoBRET Tracer Reagent. Thoroughly mix plate on an orbital shaker for 15 seconds at 900 rpm. Incubate the plate at 37° C., 5% CO₂ for 2 hours. Remove plate from incubator and equilibrate to room temperature for 15 minutes. Prepare 3× Complete Substrate plus Inhibitor Solution in Opti-MEM I Reduced Serum Medium, no phenol red, just before measuring BRET. Mix gently by inversion 5-10 times in a conical tube. Add 50 L of 3× Complete Substrate plus Inhibitor Solution to each well of the 96-well plate. Incubate for 2-3 minutes at room temperature. Measure donor emission wavelength (e.g., 450 nm) and acceptor emission wavelength (e.g., 610 nm) using NanoBRET Assay-compatible luminometer.

To generate raw BRET ratio values, divide the acceptor emission value (e.g., 610 nm) by the donor emission value (e.g., 450 nm) for each sample.

Convert raw BRET units to milliBRET units (mBU) by multiplying each raw BRET value by 1,000.

$$NanoBRET™ ratio$$

$$BRET Ratio = [ {Acceptor}_{sample} / {Donor}_{sample} ] \times 1 , 000 \qquad Equation 1$$

$$NanoBRET™ ratio equation , including optional background correction$$

$$BRET Ratio = [ ( {Acceptor}_{sample} / {Donor}_{sample} ) - {Acceptor}_{no - tracer control} / {Donor}_{no - tracer control} ) ] \times 1000 \qquad Equation 2$$

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula (Ia): 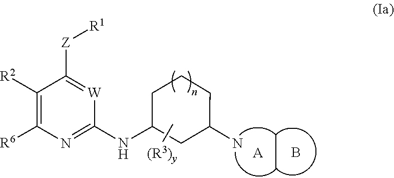
or a pharmaceutically acceptable salt thereof, wherein:
n is 0 or 1;
y is 0, 1, 2, 3, 4, 5, 6, 7, 8, or 9;
W is CH or N;
X is CR³ or N;
Z is NR^{N}, O, S, or absent;
Ring A and Ring B together form a fused bicycle;
Ring A is a 5-membered heteroaryl, which is optionally substituted with 1 or 2 independently selected R⁴ substituents;
or, Ring A is a 5-membered heterocycloalkyl, which is optionally substituted with 1, 2, 3, 4, 5, or 6 independently selected R⁴ substituents;
Ring B is phenyl or a 6-membered heteroaryl, each of which is optionally substituted with 1, 2, 3, or 4 independently selected R⁵ substituents;
R¹ is a 5-membered heteroaryl, which is optionally substituted with 1, 2, 3 or 4 independently selected R^{1A} substituents;
R^{N} is selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{1A} is independently selected from D, halo, CN, NO₂, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R1B substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j11} and R^{k11} is independently selected from OH, C₁₋₆ alkoxy, and C₁₋₆ haloalkoxy;
or, any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{e12}S(O)NR^{c12}R^{d12}, NR^{e12}S(O)R^{b12}, NR^{e12}S(O)₂R^{b12}, NR^{e12}S(O)(═NR^{e12})R^{b12}, NR^{e12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
or, any R^{c12} and R^{d12}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected Ric substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1C} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or, any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13}, NHOR^{a13}, C(O)R^{b13}, C(O)NR^{c13}R^{d13}, C(O)NR^{c13}(OR^{a13}), C(O)OR^{a13}, OC(O)R^{b13}, OC(O)NR^{c13}R^{d13} NR^{c13}R^{d13}, NR^{c13}NR^{c13}R^{d13}, NR^{c13}C(O)R^{b13}, NR^{c13}C(O)OR^{a13}, NR^{c13}C(O)NR^{c13}R^{d13}, C(═NR^{e13})R^{b13}, C(═NR^{e13})NR^{c13}R^{d13}, NR^{c13}C(═NR^{c13})NR^{c13}R^{d13}, NR^{c13}C(═NR^{e13})R^{b13}, NR^{c13}S(O)NR^{c13}R^{d13}, NR^{c13}S(O)R^{b13}, NR^{c13}S(O)₂R^{b13}, NR^{c13}S(O)(═NR^{e13})R^{b13}, NR^{c13}S(O)₂NR^{c13}R^{d13}, S(O)R^{b13}, S(O)NR^{c3}R^{d13}, S(O)₂R^{b13}, S(O)₂NR^{c13}R^{d13}, OS(O)(═NR^{e13})R^{b13}, OS(O)₂R^{b13}, S(O)(═NR^{e13})R^{b13}, SF₅, P(O)R^{f13}R^{g13}, OP(O)(OR^{h13})(ORⁱ¹³), P(O)(OR^{h13})(ORⁱ¹³), and BR^{j13}R^{k13}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a13}, R^{c13}, and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c13} and R^{d13} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f13} and R^{g13} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h13} and Rⁱ¹³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j13} and R^{k13} is independently selected from OH, C₁₋₆ alkoxy, and C₁₋₆ haloalkoxy;
or, any R^{j13} and R^{k13} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, N₁R^{c2}C(O)NR^{c2}R^{d2}, C(═NR^{e2})R^{b2}, C(═NR^{e2}NR^{c2}R^{d2}, NR^{c2}C(═N R^{e2}NR^{c2}R^{d2}, NR^{e2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2} NR^{c2}S(O)₂NR^{c2}R^{d2}, S(O)R^{b2}, S(O)NR^{c2}R^{d2}, S(O)₂R^{b2}, S(O)₂NR^{e2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or, any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})R^{e21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a21}, R^{c21}, and R^{d21}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c21} and R^{d21}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b21}, is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or, any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R³ is independently selected from H, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, S(O)(═NR^{e3})R^{b3}, SF₅, P(O)R^{f3}R^{g3}, OP(O)(OR^{h3})(ORⁱ³), P(O)(OR^{h3})(ORⁱ³), and BR^{j3}R^{k3}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3A} substituents;
or, any R^{c3} and R^{d3} attached to the same N atom, together with the N atom to which they are attached, form a 4-10 membered heterocycloalkyl group, which is optionally substituted with 1, 2, 3, or 4 independently selected R^{3A} substituents;
each R^{b3} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j3} and R^{k3} is independently selected from OH, C₁₋₆ alkoxy, and C₁₋₆ haloalkoxy;
or, any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a31}, SR^{a31} NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c31}C(═NR^{e31})R^{b31}, NR^{c31}S(O)NR^{c31}R^{d31}, NR^{c31}S(O)R^{b31} NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, S(O)(═NR^{e31})R^{b31}, SF₅, P(O)R^{f31}R^{g31}, OP(O)(OR^{h31})(ORⁱ³¹), P(O)(OR^{h31})(ORⁱ³¹), and BR^{j31}R^{k31}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁-4 alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
or, any R^{c31} and R^{d31} attached to the same N atom, together with the N atom to which they are attached, form a 4-7 membered heterocycloalkyl group, which is optionally substituted with 1, 2, 3, or 4 independently selected R^{3B} substituents;
each R^{b31} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3B} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f31} and R^{g31} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h31} and Rⁱ³¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j31} and R^{k31} is independently selected from OH, C₁₋₆ alkoxy, and C₁₋₆ haloalkoxy;
or, any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a32}, SR^{a32}, NHOR^{a32}, C(O)R^{b32}, C(O)NR^{c32}R^{d32}, C(O)NR^{c32}(OR^{a32}), C(O)OR^{a32}, OC(O)R^{b32}, OC(O)NR^{c32}R^{d32} NR^{c32}R^{d32} NR^{c32}NR^{c32}R^{d32}, NR^{c32}C(O)R^{b32}, NR^{c32}C(O)OR^{a32} NR^{c32}C(O)NR^{c32}R^{d32}, C(═NR^{e32})R^{b32}, C(═NR^{e32})NR^{c32}R^{d32}, NR^{c32}C(═NR^{e32})NR^{c32}R^{d32} NR^{c32}C(═NR^{e32})R^{b32}, NR^{c32}S(O)NR^{c32}R^{d32} NR^{c32}S(O)R^{b32}, NR^{c32}S(O)₂R^{b32}, NR^{c32}S(O)(═NR^{e32})R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, OS(O)(═NR^{e32})R^{b32}, OS(O)₂R^{b32}, S(O)(═NR^{e32})R^{b32}, SF₅, P(O)R^{f32}R^{g32}, OP(O)(OR^{h32})(ORⁱ³²), P(O)(OR^{h32})(ORⁱ³²), and BR^{j32}R^{k32}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a32}, R^{c32}, and R^{d32} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c32} and R^{d32} attached to the same N atom, together with the N atom to which they are attached, form a 4-7 membered heterocycloalkyl group, which is optionally substituted with 1, 2, 3, or 4 independently selected R^{G} substituents;
each R^{b32} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e32} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f32} and R^{g32} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h32} and Rⁱ³² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j32} and R^{k32} is independently selected from OH, C₁₋₆ alkoxy, and C₁₋₆ haloalkoxy;
or, any R^{j32} and R^{k32} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁴ is independently selected from oxo, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a4} SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4} NR^{c4}NR^{c4}R^{d4} NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4} NR^{c4}C(═NR^{e4})NR^{c4}R^{d4} NR^{c4}C(═NR^{e4})R^{b4}, NR^{c4}S(O)NR^{c4}R^{d4} NR^{c4}S(O)R^{b4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, S(O)(═NR^{e4})R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c4} and R^{d4} attached to the same N atom, together with the N atom to which they are attached, form a 4-10 membered heterocycloalkyl group, which is optionally substituted with 1, 2, 3, or 4 independently selected R^{4A} substituents;
each R^{b4} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{c4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f4} and R^{g4} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h4} and Rⁱ⁴ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a41} SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41} NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41} NR^{c41}S(O)NR^{c41}R^{d41}, NR^{c41}S(O)R^{b41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41} OS(O)₂R^{b41}, S(O)(═NR^{e41})R^{b41}, SF₄, p(O)R^{f41}R^{g41}, OP(O)(OR^{h41})(ORⁱ⁴¹), P(O)(OR^{h41})(ORⁱ⁴¹), and BR^{j41}R^{k41}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4B} substituents;
each R^{c41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f41} and R^{g41} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h41} and Rⁱ⁴¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j41} and R^{k41} is independently selected from OH, C₁₋₆ alkoxy, and C₁₋₆ haloalkoxy;
or, any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R⁴², OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)NR^{c42}R^{d42}, NR^{c42}S(O)R^{b42}, NR^{c42}S(O)₂R^{b42}, NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, OS(O)₂R^{b42}, S(O)(═NR^{e42})R^{b42}, SF₅, P(O)R^{f42}R^{g42}, OP(O)(OR^{h42})(ORⁱ⁴²), P(O)(OR^{h42})(ORⁱ⁴²), and BR^{j42}R^{k42}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c42} and R^{d42} attached to the same N atom, together with the N atom to which they are attached, form a 4-7 membered heterocycloalkyl group, which is optionally substituted with 1, 2, 3, or 4 independently selected R^{G} substituents;
each R^{b42} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f42} and R^{g42} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h42} and Rⁱ⁴² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j42} and R^{k42} is independently selected from OH, C₁₋₆ alkoxy, and C₁₋₆ haloalkoxy;
or, any R^{j42} and R^{k42} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}(O)R^{b5}, NR^{c5}(O)OR^{a5}, NR^{c5}(O)NR^{c5}R^{d5}, C(═NR^{e5})R^{b5}, C(═NR^{e5})NR^{c5}R^{d5}, NR^{c5}(═NR^{e5})NR^{c5}R^{d5}, NR^{c5}(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{4A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. A compound of Formula (I): 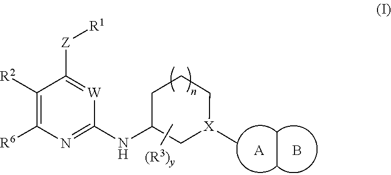
or a pharmaceutically acceptable salt thereof, wherein:
n is 0 or 1;
y is 0, 1, 2, 3, 4, 5, 6, 7, 8, or 9;
W is CH or N;
X is CR³ or N;
Z is NR^{N}, O, S, or absent;
the moiety 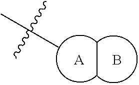
is selected from: 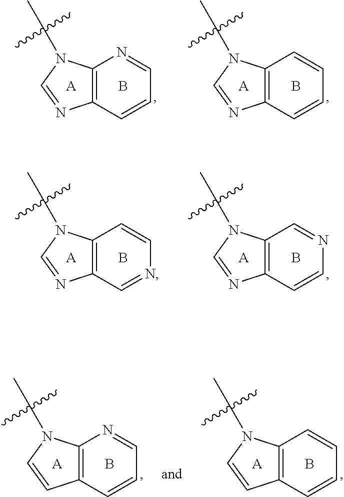
wherein Ring A is optionally substituted with 1 or 2 independently selected R⁴ substituents; and Ring B is optionally substituted with 1 or 2 independently selected R⁵ substituents;
R¹ is a 5-membered heteroaryl, which is optionally substituted with 1, 2, 3 or 4 independently selected R^{1A} substituents;
R^{N} is selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{1A} is independently selected from D, halo, CN, NO₂, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1B} substituents;
or, any two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R1B substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R1B substituents;
each R^{b11} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j11} and R^{k11} is independently selected from OH, C₁₋₆ alkoxy, and C₁₋₆ haloalkoxy;
or, any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{e12}S(O)NR^{c12}R^{d12}, NR^{e12}S(O)R^{b12}, NR^{e12}S(O)₂R^{b12}, NR^{e12}S(O)(═NR^{e12})R^{b12}, NR^{e12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
or, any R^{c12} and R^{d12}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected Ric substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1C} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or, any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13}, NHOR^{a13}, C(O)R^{b13}, C(O)NR^{c13}R^{d13}, C(O)NR^{c13}(OR^{a13}), C(O)OR^{a13}, OC(O)R^{b13}, OC(O)NR^{c13}R^{d13} NR^{c13}R^{d13}, NR^{c13}NR^{c13}R^{d13}, NR^{c13}C(O)R^{b13}, NR^{c13}C(O)OR^{a1}, NR^{c13}C(O)NR^{c3}R^{d13}, C(═NR^{e3})R^{b13}, C(═NR^{e13})NR^{c13}R^{d13}, NR^{c13}C(═NR^{e13})NR^{c13}R^{d13}, NR^{c13}C(═NR^{e13})R^{b13}, NR^{c13}S(O)NR^{c13}R^{d13}, NR^{c13}S(O)R^{b13}, NR^{c13}S(O)₂R^{b13}, NR^{c13}S(O)(═NR^{e13})R^{b13}, NR^{c13}S(O)₂NR^{c13}R^{d13}, S(O)R^{b13}, S(O)NR^{c3}R^{d13}, S(O)₂R^{b13}, S(O)₂NR^{c13}R^{d13}, OS(O)(═NR^{e13})R^{b13}, OS(O)₂R^{b13}, S(O)(═NR^{e13})R^{b13}, SF₅, P(O)R^{f13}R^{g13}, OP(O)(OR^{h13})(ORⁱ¹³), P(O)(OR^{h13})(ORⁱ¹³), and BR^{j13}R^{k13}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a13}, R^{c13}, and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c13} and R^{d13} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f13} and R^{g13} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h13} and Rⁱ³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j13} and R^{k13} is independently selected from OH, C₁₋₆ alkoxy, and C₁₋₆ haloalkoxy;
or, any R^{j13} and R^{k13} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, N₁R^{c2}C(O)NR^{c2}R^{d2}, C(═NR^{e2})R^{b2}, C(═NR^{e2})NR^{c2}R^{d2}, NR^{c2}C(═N R^{e2}NR^{c2}R^{d2}, NR^{e2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2} NR^{c2}S(O)₂NR^{c2}R^{d2}, S(O)R^{b2}, S(O)NR^{c2}R^{d2}, S(O)₂R^{b2}, S(O)₂NR^{e2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or, any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{e21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{e21}R^{d21}, NR^{c21}C(═NR^{e21})NR^{c21}R^{d21}, NR^{e21}C(═NR^{e21})R^{b21}, NR^{c21}S(O)NR^{e21}R^{d21}, NR^{e21}S(O)R^{b21}, NR^{e21}S(O)₂R^{b21}, NR^{c21}S(O)(═NR^{e21})R^{b21}, NR^{e21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}Rⁱ²¹, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a21}, R^{c21}, and R^{d21}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c21} and R^{d21}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or, any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R³ is independently selected from H, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR³, SR³, NHOR^{a3}, C(O)R^{b3}, C(O)NR^{c3}R^{d3}, C(O)NR^{c3}(OR^{a3}), C(O)OR^{a13}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, S(O)(═NR^{e3})R^{b3}, SF₅, P(O)R^{f3}R^{g3}, OP(O)(OR^{h3})(ORⁱ³), P(O)(OR^{h3})(ORⁱ³), and BR^{j3}R3; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3A} substituents;
or, any R^{c3} and R^{d3} attached to the same N atom, together with the N atom to which they are attached, form a 4-10 membered heterocycloalkyl group, which is optionally substituted with 1, 2, 3, or 4 independently selected R^{3A} substituents;
each R^{b3} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j3} and R^{k3} is independently selected from OH, C₁₋₆ alkoxy, and C₁₋₆ haloalkoxy;
or, any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a31}, SR^{a31} NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31} NR^{c31}C(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c31}C(═NR^{e31})R^{b31} NR^{c31}S(O)NR^{c3}R^{d31}, NR^{c31}S(O)R^{b31} NR^{c31}S(O)₂R^{b31}, NR^{c31}S(O)(═NR^{e3})R^{b31} NR^{c31}S(O)₂NR^{c31}R^{d31}, S(O)R^{b31}, S(O)NR^{c3}R^{d31}, S(O)₂R^{b31}, S(O)₂NR^{c31}R^{d31}, OS(O)(═NR^{e3})R^{b31} OS(O)₂R^{b31}, S(O)(═NR^{e3})R^{b31}, SF₅, P(O)R^{f31}R^{g31}, OP(O)(OR^{h31})(ORⁱ³¹), P(O)(OR^{h31})(ORⁱ³¹), and BR^{j31}R^{k31}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁-4 alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{3B} substituents;
or, any R^{c31} and R^{d31} attached to the same N atom, together with the N atom to which they are attached, form a 4-7 membered heterocycloalkyl group, which is optionally substituted with 1, 2, 3, or 4 independently selected R^{3B} substituents;
each R^{b31} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{3B} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f31} and R^{g31} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h31} and Rⁱ³¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j31} and R^{k31} is independently selected from OH, C₁₋₆ alkoxy, and C₁₋₆ haloalkoxy;
or, any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a32}, SR^{a32}, NHOR^{a32}, C(O)R^{b32}, C(O)NR^{c32}R^{d32}, C(O)NR^{c32}(OR^{a32}), C(O)OR^{a32}, OC(O)R^{b32}, OC(O)NR^{c32}R^{d32} NR^{c32}R^{d32} NR^{c32}NR^{c32}R^{d32}, NR^{c32}C(O)R^{b32}, NR^{c32}C(O)OR^{a32}, NR^{c32}C(O)NR^{c32}R^{d32}, C(═NR^{e32})R^{b32}, C(═NR^{e32})NR^{c32}R^{d32}, NR^{c32}C(═NR^{e32})NR^{c32}R^{d32} NR^{c32}C(═NR^{e32})R^{b32}, NR^{c32}S(O)NR^{c32}R^{d32} NR^{c32}S(O)R^{b32}, NR^{c32}S(O)₂R^{b32}, NR^{c32}S(O)(═NR^{e32})R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, OS(O)(═NR^{e32})R^{b32}, OS(O)₂R^{b32}, S(O)(═NR^{e32})R^{b32}, SF₅, P(O)R^{f32}R^{g32}, OP(O)(OR^{h32})(ORⁱ³²), P(O)(OR^{h32})(ORⁱ³²), and BR^{j32}R^{k32}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a32}, R^{c32}, and R^{d32} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c32} and R^{d32} attached to the same N atom, together with the N atom to which they are attached, form a 4-7 membered heterocycloalkyl group, which is optionally substituted with 1, 2, 3, or 4 independently selected R^{G} substituents;
each R^{b32} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e32} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f32} and R^{g32} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h32} and Rⁱ³² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j32} and R^{k32} is independently selected from OH, C₁₋₆ alkoxy, and C₁₋₆ haloalkoxy;
or, any R^{j32} and R^{k32} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁴ is independently selected from oxo, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a4} SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4} NR^{c4}NR^{c4}R^{d4} NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4} NR^{c4}C(═NR^{e4})NR^{c4}R^{d4} NR^{c4}C(═NR^{e4})R^{b4}, NR^{c4}S(O)NR^{c4}R^{d4} NR^{c4}S(O)R^{b4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, S(O)(═NR^{e4})R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c4} and R^{d4} attached to the same N atom, together with the N atom to which they are attached, form a 4-10 membered heterocycloalkyl group, which is optionally substituted with 1, 2, 3, or 4 independently selected R^{4A} substituents;
each R^{b4} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{c4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f4} and R^{g4} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h4} and Rⁱ⁴ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a41}, SR^{a41} NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41} NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41} NR^{c41}S(O)NR^{c41}R^{d41}, NR^{c41}S(O)R^{b41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{c41})R^{b41} OS(O)₂R^{b41}, S(O)(═NR^{e41})R^{b41}, SF₄, p(O)R^{f41}R^{g41}, OP(O)(OR^{h41})(ORⁱ⁴¹), P(O)(OR^{h41})(ORⁱ⁴¹), and BR^{j41}R^{k41}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁-4 alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f41} and R^{g41} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h41} and Rⁱ⁴¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j41} and R^{k41} is independently selected from OH, C₁₋₆ alkoxy, and C₁₋₆ haloalkoxy;
or, any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)NR^{c42}R^{d42}, NR^{c42}S(O)R^{b42}, NR^{c42}S(O)₂R^{b42}, NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, OS(O)₂R^{b42}, S(O)(═NR^{e42})R^{b42}, SF₅, p(O)R^{f42}R^{g42}, OP(O)(OR^{h42})(ORⁱ⁴²), P(O)(OR^{h42})(ORⁱ⁴²), and BR^{j42}R^{k42}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c42} and R^{d42} attached to the same N atom, together with the N atom to which they are attached, form a 4-7 membered heterocycloalkyl group, which is optionally substituted with 1, 2, 3, or 4 independently selected R^{G} substituents;
each R^{b42} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f42} and R^{g42} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h42} and Rⁱ⁴² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j42} and R^{k42} is independently selected from OH, C₁₋₆ alkoxy, and C₁₋₆ haloalkoxy;
or, any R^{j42} and R^{k42} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}(O)R^{b5}, NR^{c5}(O)OR^{a5}, NR^{c5}(O)NR^{c5}R^{d5}, C(═NR^{e5})R^{b5}, C(═NR^{e5})NR^{c5}R^{d5}, NR^{c5}(═NR^{e5})NR^{c5}R^{d5}, NR^{c5}(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{4A} substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{4A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{4A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

3. The compound of claim 2, or a pharmaceutically acceptable salt thereof, wherein W is N.

4. The compound of claim 2, or a pharmaceutically acceptable salt thereof, wherein X is CH.

5. The compound of claim 2, or a pharmaceutically acceptable salt thereof, wherein Z is absent.

6. The compound of claim 2, or a pharmaceutically acceptable salt thereof, wherein R¹ is pyrazolyl, imadazolyl, or triazolyl, each of which is optionally substituted with 1, 2, 3, or 4 independently selected R^{1A} substituents.

7. The compound of claim 2, or a pharmaceutically acceptable salt thereof, wherein each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)N R^{c11}R^{d11}, NR^{c11}S(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents; or
two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents; and
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, which are each optionally substituted with 1, 2, 3, or 4 independently selected R¹ substituents.

8. The compound of claim 2, or a pharmaceutically acceptable salt thereof, wherein each R^{1A} is independently selected from CN, C₁₋₆ alkyl, C₁₋₆ haloalkyl, and C₃₋₇ cycloalkyl, wherein said C₁₋₆ alkyl, C₁₋₆ haloalkyl, and C₃₋₇ cycloalkyl are each optionally substituted with 1, 2, 3, or 4 independently selected R^{1B} substituents.

9. The compound of am claim 2, or a pharmaceutically acceptable salt thereof, wherein two R^{1A} substituents, together with the carbon or nitrogen atoms to which they are attached, form a 5- or 6-membered heterocycloalkyl ring, which is optionally substituted with 1, 2, 3, or 4 independently selected R^{1B} substituents.

10. The compound of claim 2, or a pharmaceutically acceptable salt thereof, wherein each R^{1B} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12} SR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}C(O)R^{b12}, R^{c12}C(O)OR^{a12}, NR^{c12}C(O)NR^{c12}R^{d12}, NR^{c12}S(O)NR^{c12}R^{d12}, NR^{c12}S(O)R^{b12}, NR^{c12}S(O)₂R^{b12}, NR^{c12}S(O)₂NR^{c12}R^{d12}, S(O)R^{b12}, S(O)NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected Ric substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
or, any R^{c12} and R^{d12}, attached to the same N atom, together with the N atom to which they are attached, form a 4-7 membered heterocycloalkyl group; and
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

11. The compound of claim 2, or a pharmaceutically acceptable salt thereof, wherein each R^{1B} is independently OR^{a12}, and each R^{a12} is independently selected from H and C₁₋₆ alkyl.

12. The compound of nm claim 2, or a pharmaceutically acceptable salt thereof, wherein R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
or, any R^{c2} and R^{d2} attached to the same N atom, together with the N atom to which they are attached, form a 4-10 membered heterocycloalkyl group; and
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl.

13. The compound of claim 2, or a pharmaceutically acceptable salt thereof, wherein each R^{2A} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

14. The compound of claim 2, or a pharmaceutically acceptable salt thereof, wherein R² is selected from H, halo, CN, C₁₋₆ alkyl, and C₁₋₆ haloalkyl.

15. The compound of claim 2, or a pharmaceutically acceptable salt thereof, wherein R² is selected from Cl, CN, and CF₃.

16. The compound of claim 2, or a pharmaceutically acceptable salt thereof, wherein y is 0, 1, or 2.

17. The compound of claim 2, or a pharmaceutically acceptable salt thereof, wherein y is 0.

18. The compound of claim 2, or a pharmaceutically acceptable salt thereof, wherein n is 0.

19. The compound of claim 2, or a pharmaceutically acceptable salt thereof, wherein n is 1.

20. The compound of claim 2, or a pharmaceutically acceptable salt thereof, wherein each R⁴ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

21. The compound of claim 2, or a pharmaceutically acceptable salt thereof, wherein each R⁴ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

22. The compound of claim 2, or a pharmaceutically acceptable salt thereof, wherein each R^{4A} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

23. The compound of claim 2, or a pharmaceutically acceptable salt thereof, wherein each R⁵ is independently selected from halo, NO₂, CN, C₁₋₆ alkyl, C₂₋₆ alkenyl, C₂₋₆ alkynyl, C₁₋₆ haloalkyl, OR^{a5}, C(O)R^{b5}, and C(O)OR^{a5}, wherein said C₁₋₆ alkyl, C₂₋₆ alkenyl, C₂₋₆ alkynyl, and C₁₋₆ haloalkyl are each optionally substituted by 1, 2, 3, or 4 independently selected R^{4A} substituents;
each R^{a5} and R^{b5} is independently selected from H, C₁₋₆ alkyl, C₁₋₆ haloalkyl, C₂₋₆ alkenyl, and C₂₋₆ alkynyl.

24. The compound of claim 2, or a pharmaceutically acceptable salt thereof, wherein each R⁵ is independently selected from CN, C₁₋₆ alkyl, C₁₋₆ haloalkyl, OR^{a5}, and C(O)OR^{a5}; and
each R^{a5} and R^{b5} is independently selected from H and C₁₋₆ alkyl.

25. The compound of claim 2, or a pharmaceutically acceptable salt thereof, wherein R⁶ is selected from H, D, OH, NO₂, CN, halo, C₁₋₃ alkyl, C₂₋₃ alkenyl, C₂₋₃ alkynyl, and C₁₋₃ haloalkyl.

26. The compound of claim 2, or a pharmaceutically acceptable salt thereof, wherein R⁶ is H.

27. The compound of claim 2, or a pharmaceutically acceptable salt thereof, wherein:
n is 0 or 1;
y is 0, 1, or 2;
W is N;
X is CH or N;
Z is NH, O, S, or absent;
R¹ is pyrazolyl, imadazolyl, or triazolyl, each of which is optionally substituted with 1, 2, 3, or 4 independently selected R^{1A} substituents;
each R^{1A} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}NR^{c11}R^{d11}, NR^{c11}C(O)R^{b11}, NR^{c11}C(O)OR^{a11}, NR^{c11}C(O)NR^{c11}R^{d11}, NR^{c11}S(O)NR^{c11}R^{d11}, NR^{c11}S(O)R^{b11}, NR^{c11}S(O)₂R^{b11}, NR^{c11}S(O)₂NR^{c11}R^{d11} S(O)R^{b11}, S(O)NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents; or
two R^{1A} substituents, together with the carbon or nitrogen atoms to which they are attached, form a 5- or 6-membered cycloalkyl or a 5- or 6-membered heterocycloalkyl ring, each of which is optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R¹ substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents; and
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl, which are each optionally substituted with 1, 2, 3, or 4 independently selected R^{1B} substituents;
each R^{1B} is independently selected from halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12} NR^{c12}R^{d12}, NR^{c12}C(O)R^{b12}, NR^{c12}C(O)OR^{a12}, NR^{c12}C(O)NR^{c12}R^{d12}, NR^{c12}S(O)NR^{c12}R^{d12}, NR^{c12}S(O)R^{b12}, NR^{c12}S(O)₂R^{b12}, NR^{c12}S(O)₂NR^{c12}R^{d12} S(O)R^{b12}, S(O)NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1C} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
or, any R^{c12} and R^{d12}, attached to the same N atom, together with the N atom to which they are attached, form a 4-7 membered heterocycloalkyl group; and
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl;
each R^{1C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a13}, SR^{a13}, C(O)R^{b13}, C(O)NR^{c13}R^{d13}, C(O)NR^{c13}(OR^{a13}), C(O)OR^{a13}, OC(O)R^{b13}, OC(O)NR^{c13}R^{d13} NR^{c13}R^{d13}, NR^{c13}C(O)R^{b13}, NR^{c13}C(O)OR^{a13} NR^{c13}C(O)NR^{c13}R^{d13}, NR^{c13}S(O)NR^{c13}R^{d13}, NR^{c13}S(O)R^{b13}, NR^{c13}S(O)₂R^{b13}, NR^{c13}S(O)₂NR^{c13}R^{d13}, S(O)R^{b13}, S(O)NR^{c13}R^{d13}, S(O)₂R^{b13}, and S(O)₂NR^{c13}R^{d13};
each R^{a13}, R^{c13}, and R^{d13} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
or, any R^{c13} and R^{d13} attached to the same N atom, together with the N atom to which they are attached, form a 4-7 membered heterocycloalkyl group;
each R^{b13} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
R² is selected from H, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
or, any R^{c2} and R^{d2} attached to the same N atom, together with the N atom to which they are attached, form a 4-10 membered heterocycloalkyl group; and
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{e}21R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)NR^{c21}R^{d21}, NR^{c21}S(O)R^{b21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, and S(O)₂NR^{c21}R^{d21};
each R^{a21}, R^{c21}, and R^{d21}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
or, any R^{c21} and R^{d21}, attached to the same N atom, together with the N atom to which they are attached, form a 4-7 membered heterocycloalkyl group;
each R^{b21}, is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R⁴ is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R⁵ is independently selected from D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}(O)R^{b5}, NR^{c5}(O)OR^{a5}, N R(O)NR^{c5}R^{d5}, NR^{c5}S(O)—NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl;
or, any R^{c5} and R^{d5} attached to the same N atom, together with the N atom to which they are attached, form a 4-10 membered heterocycloalkyl group;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, and 5-10 membered heteroaryl; and
R⁶ is selected from H and C₁₋₃ alkyl.

28. The compound of claim 2, which is:
(a) a compound of Formula (II): 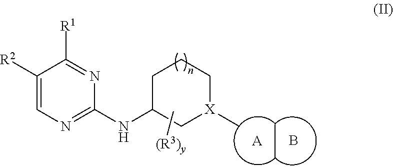
or a pharmaceutically acceptable salt thereof; or
(b) a compound of Formula (III): 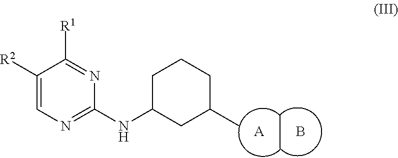
or a pharmaceutically acceptable salt thereof; or
(c) a compound of Formula (IV): 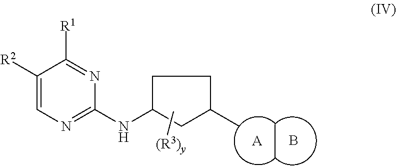
or a pharmaceutically acceptable salt thereof; or
(d) a compound of Formula (V): 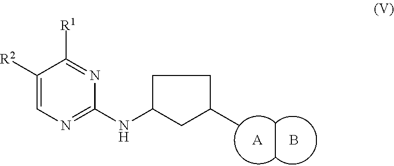
or a pharmaceutically acceptable salt thereof.

29. The compound of claim 2, which is selected from:
3-((1S,3R)-3-((4-(1-(2,2-difluoroethyl)-1H-pyrazol-4-yl)-5-(trifluoromethyl)pyrimidin-2-yl)amino)cyclohexyl)-3H-imidazo[4,5-b]pyridine-6-carbonitrile;
3-((1S,3R)-3-((4-(1-(2,2-difluoroethyl)-1H-pyrazol-4-yl)-5-(trifluoromethyl)pyrimidin-2-yl)amino)cyclopentyl)-3H-imidazo[4,5-b]pyridine-6-carbonitrile;
2-(((1R,3S)-3-(1H-benzo[d]imidazol-1-yl)cyclohexyl)amino)-4-(1-(2,2-difluoroethyl)-1H-pyrazol-4-yl)pyrimidine-5-carbonitrile;
4-(1-(2,2-difluoroethyl)-1H-pyrazol-4-yl)-5-(trifluoromethyl)-N-((1R,3S)-3-(6-(trifluoromethyl)-3H-imidazo[4,5-b]pyridin-3-yl)cyclohexyl)pyrimidin-2-amine;
methyl 3-((1S,3R)-3-((4-(1-(2,2-difluoroethyl)-1H-pyrazol-4-yl)-5-(trifluoromethyl)pyrimidin-2-yl)amino)cyclohexyl)-3H-imidazo[4,5-b]pyridine-5-carboxylate;
4-(1-(2,2-difluoroethyl)-1H-pyrazol-4-yl)-N-((1R,3S)-3-(5-methoxy-3H-imidazo[4,5-b]pyridin-3-yl)cyclohexyl)-5-(trifluoromethyl)pyrimidin-2-amine;
N-((1R,3S)-3-(1H-imidazo[4,5-c]pyridin-1-yl)cyclohexyl)-4-(1-(2,2-difluoroethyl)-1H-pyrazol-4-yl)-5-(trifluoromethyl)pyrimidin-2-amine;
4-(1-(2,2-difluoroethyl)-1H-pyrazol-4-yl)-2-(((1R,3S)-3-(7-methyl-3H-imidazo[4,5-b]pyridin-3-yl)cyclohexyl)amino)pyrimidine-5-carbonitrile;
3-((1S,3R)-3-((5-cyano-4-(1-(2,2-difluoroethyl)-1H-pyrazol-4-yl)pyrimidin-2-yl)amino)cyclohexyl)-3H-imidazo[4,5-b]pyridine-6-carbonitrile;
N-((1R,3S)-3-(3H-imidazo[4,5-c]pyridin-3-yl)cyclohexyl)-4-(1-(2,2-difluoroethyl)-1H-pyrazol-4-yl)-5-(trifluoromethyl)pyrimidin-2-amine;
2-(((1R,3S)-3-(3H-imidazo[4,5-b]pyridin-3-yl)cyclohexyl)amino)-4-(1-(2,2-difluoroethyl)-1H-pyrazol-4-yl)pyrimidine-5-carbonitrile;
N-((1R,3S)-3-(3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine;
1-((1S,3R)-3-((5-cyano-4-(1-(2,2-difluoroethyl)-1H-pyrazol-4-yl)pyrimidin-2-yl)amino)cyclohexyl)-1H-benzo[d]imidazole-7-carbonitrile;
3-((1S,3R)-3-((5-chloro-4-(1-(2,2-difluoroethyl)-1H-pyrazol-4-yl)pyrimidin-2-yl)amino)cyclohexyl)-3H-imidazo[4,5-b]pyridine-6-carbonitrile;
1-((1S,3R)-3-((4-(1-(2,2-difluoroethyl)-1H-pyrazol-4-yl)-5-(trifluoromethyl)pyrimidin-2-yl)amino)cyclohexyl)-1H-pyrrolo[2,3-b]pyridine-5-carbonitrile;
1-((1S,3R)-3-((4-(1-(2,2-difluoroethyl)-1H-pyrazol-4-yl)-5-(trifluoromethyl)pyrimidin-2-yl)amino)cyclohexyl)-1H-indole-5-carbonitrile;
2-(((1R,3S)-3-(3H-imidazo[4,5-b]pyridin-3-yl)cyclohexyl)amino)-4-(1,5-dimethyl-1H-pyrazol-4-yl)pyrimidine-5-carbonitrile;
2-(((1R,3S)-3-(3H-imidazo[4,5-b]pyridin-3-yl)cyclohexyl)amino)-4-(5,6-dihydro-4H-pyrrolo[1,2-b]pyrazol-3-yl)pyrimidine-5-carbonitrile;
2-(((1R,3S)-3-(3H-imidazo[4,5-b]pyridin-3-yl)cyclohexyl)amino)-4-(1,3-dimethyl-1H-pyrazol-4-yl)pyrimidine-5-carbonitrile;
2-(((1R,3S)-3-(3H-imidazo[4,5-b]pyridin-3-yl)cyclohexyl)amino)-4-(5-cyano-1-methyl-1H-pyrazol-4-yl)pyrimidine-5-carbonitrile;
2-(((1R,3S)-3-(3H-imidazo[4,5-b]pyridin-3-yl)cyclohexyl)amino)-4-(3-cyclopropyl-1H-pyrazol-4-yl)pyrimidine-5-carbonitrile;
2-(((1R,3S)-3-(3H-imidazo[4,5-b]pyridin-3-yl)cyclohexyl)amino)-4-(1-methyl-1H-pyrazol-5-yl)pyrimidine-5-carbonitrile;
2-(((1R,3S)-3-(3H-imidazo[4,5-b]pyridin-3-yl)cyclohexyl)amino)-4-(1-methyl-5-(trifluoromethyl)-1H-pyrazol-4-yl)pyrimidine-5-carbonitrile;
2-(((1R,3S)-3-(3H-imidazo[4,5-b]pyridin-3-yl)cyclohexyl)amino)-4-(1-ethyl-1H-pyrazol-4-yl)pyrimidine-5-carbonitrile;
2-(((1R,3S)-3-(3H-imidazo[4,5-b]pyridin-3-yl)cyclohexyl)amino)-4-(6,7-dihydro-5H-pyrazolo[5,1-b][1,3]oxazin-3-yl)pyrimidine-5-carbonitrile;
2-(((1R,3S)-3-(3H-imidazo[4,5-b]pyridin-3-yl)cyclohexyl)amino)-4-(5-(hydroxymethyl)-1-methyl-1H-pyrazol-4-yl)pyrimidine-5-carbonitrile;
2-(((1R,3S)-3-(3H-imidazo[4,5-b]pyridin-3-yl)cyclohexyl)amino)-4-(1-isopropyl-1H-imidazol-4-yl)pyrimidine-5-carbonitrile;
3-((1S,3R)-3-((5-cyano-4-(1,5-dimethyl-1H-1,2,3-triazol-4-yl)pyrimidin-2-yl)amino)cyclohexyl)-3H-imidazo[4,5-b]pyridine-6-carbonitrile;
3-((1S,3R)-3-((5-cyano-4-(1,5-dimethyl-1H-imidazol-2-yl)pyrimidin-2-yl)amino)cyclohexyl)-3H-imidazo[4,5-b]pyridine-6-carbonitrile; and
3-((1S,3R)-3-((5-cyano-4-((1,5-dimethyl-1H-pyrazol-4-yl)amino)pyrimidin-2-yl)amino)cyclohexyl)-3H-imidazo[4,5-b]pyridine-6-carbonitrile;
or a pharmaceutically acceptable salt thereof.

30. The compound of claim 2, selected from:
1-((1S,3R)-3-((5-cyano-4-(1-(2,2-difluoroethyl)-1H-pyrazol-4-yl)pyrimidin-2-yl)amino)cyclohexyl)-1H-imidazo[4,5-c]pyridine-7-carbonitrile;
1-((1S,3R)-3-((5-cyano-4-(1-(2-hydroxyethyl)-1H-pyrazol-4-yl)pyrimidin-2-yl)amino)cyclohexyl)-1H-imidazo[4,5-c]pyridine-7-carbonitrile;
1-((1S,3R)-3-((5-cyano-4-(1-isopropyl-1H-pyrazol-4-yl)pyrimidin-2-yl)amino)cyclohexyl)-1H-imidazo[4,5-c]pyridine-7-carbonitrile;
1-((1S,3R)-3-((5-cyano-4-(1-cyclopropyl-1H-pyrazol-4-yl)pyrimidin-2-yl)amino)cyclohexyl)-1H-imidazo[4,5-c]pyridine-7-carbonitrile;
3-((1R,3S)-3-((5-cyano-4-(1-(2,2-difluoroethyl)-1H-pyrazol-4-yl)pyrimidin-2-yl)amino)cyclohexyl)-3H-imidazo[4,5-b]pyridine-6-carbonitrile;
2-(((1R,3S)-3-(7-chloro-5-fluoro-1H-benzo[d]imidazol-1-yl)cyclohexyl)amino)-4-(1-(2,2-difluoroethyl)-1H-pyrazol-4-yl)pyrimidine-5-carbonitrile;
3-((1S,3R)-3-((5-cyano-4-(1-(1-hydroxy-2-methylpropan-2-yl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-cyclobutyl-1H-pyrazol-4-yl)pyrimidin-2-yl)amino)cyclohexyl)-3H-imidazo[4,5-b]pyridine-6-carbonitrile;
2-(((1R,3S)-3-(7-bromo-1H-imidazo[4,5-c]pyridin-1-yl)cyclohexyl)amino)-4-(1-(2,2-difluoroethyl)-1H-pyrazol-4-yl)pyrimidine-5-carbonitrile;
2-(((1R,3S)-3-(7-bromo-5-fluoro-1H-benzo[d]imidazol-1-yl)cyclohexyl)amino)-4-(1-(2,2-difluoroethyl)-1H-pyrazol-4-yl)pyrimidine-5-carbonitrile;
2-(((1R,3S)-3-(7-bromo-1H-benzo[d]imidazol-1-yl)cyclohexyl)amino)-4-(1-(2,2-difluoroethyl)-1H-pyrazol-4-yl)pyrimidine-5-carbonitrile;
2-(((1R,3S)-3-(7-bromo-6-chloro-1H-imidazo[4,5-c]pyridin-1-yl)cyclohexyl)amino)-4-(1-(2,2-difluoroethyl)-1H-pyrazol-4-yl)pyrimidine-5-carbonitrile;
1-((1S,3R)-3-((5-cyano-4-(1-(2,2-difluoroethyl)-1H-pyrazol-4-yl)pyrimidin-2-yl)amino)cyclohexyl)-1H-benzo[d]imidazole-6-carbonitrile;
2-(((1R,3S)-3-(7-chloro-1H-imidazo[4,5-c]pyridin-1-yl)cyclohexyl)amino)-4-(1-(2,2-difluoroethyl)-1H-pyrazol-4-yl)pyrimidine-5-carbonitrile;
1-((1S,3R)-3-((5-cyano-4-(1-(2,2-difluoroethyl)-1H-pyrazol-4-yl)pyrimidin-2-yl)amino)cyclohexyl)-1H-imidazo[4,5-c]pyridine-6-carbonitrile;
3-((1S,3R)-3-((5-cyano-4-(1-(2,2-difluoroethyl)-1H-pyrazol-4-yl)pyrimidin-2-yl)amino)cyclohexyl)-3H-imidazo[4,5-b]pyridine-5-carbonitrile;
1-((1S,3R)-3-((5-cyano-4-(1-(2,2-difluoroethyl)-1H-pyrazol-4-yl)pyrimidin-2-yl)amino)cyclohexyl)-4-methyl-1H-imidazo[4,5-c]pyridine-7-carbonitrile;
1-((1S,3R)-3-((5-cyano-4-(1-(2,2-difluoroethyl)-1H-pyrazol-4-yl)pyrimidin-2-yl)amino)cyclohexyl)-5-fluoro-1H-benzo[d]imidazole-7-carbonitrile;
1-((1S,3R)-3-((5-cyano-4-(1-(2,2-difluoroethyl)-1H-pyrazol-4-yl)pyrimidin-2-yl)amino)cyclohexyl)-1H-benzo[d]imidazole-5,7-dicarbonitrile;
1-((1S,3R)-3-((4-(1-(2,2-difluoroethyl)-1H-pyrazol-4-yl)-5-(trifluoromethyl)pyrimidin-2-yl)amino)cyclohexyl)-5-(trifluoromethyl)-1H-benzo[d]imidazole-7-carbonitrile;
1-((1S,3R)-3-((4-(1-(2,2-difluoroethyl)-1H-pyrazol-4-yl)-5-(trifluoromethyl)pyrimidin-2-yl)amino)cyclohexyl)-1H-imidazo[4,5-c]pyridine-7-carbonitrile;
6-cyano-3-((1S,3R)-3-((5-cyano-4-(1-(2,2-difluoroethyl)-1H-pyrazol-4-yl)pyrimidin-2-yl)amino)cyclohexyl)-3H-imidazo[4,5-b]pyridine 4-oxide;
4-(1-(2,2-difluoroethyl)-1H-pyrazol-4-yl)-2-(((1R,3S)-3-(5-fluoro-7-methyl-1H-benzo[d]imidazol-1-yl)cyclohexyl)amino)pyrimidine-5-carbonitrile;
1-((1S,3R)-3-((5-cyano-4-(1-(2,2-difluoroethyl)-1H-pyrazol-4-yl)pyrimidin-2-yl)amino)cyclohexyl)-7-methyl-1H-benzo[d]imidazole-5-carbonitrile;
3-((1S,3R)-3-((4-(1-(2,2-difluoroethyl)-1H-pyrazol-4-yl)-5-(1-methyl-1H-pyrazol-4-yl)pyrimidin-2-yl)amino)cyclohexyl)-3H-imidazo[4,5-b]pyridine-6-carbonitrile;
3-((1S,3R)-3-((4-(1-(2,2-difluoroethyl)-1H-pyrazol-4-yl)-5-(1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-(2-hydroxy-2-methylpropyl)-1H-pyrazol-4-yl)pyrimidin-2-yl)amino)cyclohexyl)-3H-imidazo[4,5-b]pyridine-6-carbonitrile;
3-((1S,3R)-3-((4-(1-(azetidin-3-yl)-1H-pyrazol-4-yl)-5-cyanopyrimidin-2-yl)amino)cyclohexyl)-3H-imidazo[4,5-b]pyridine-6-carbonitrile;
3-((1S,3R)-3-((5-cyano-4-(1-(2-morpholinoethyl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-methyl-1H-imidazol-4-yl)pyrimidin-2-yl)amino)cyclohexyl)-3H-imidazo[4,5-b]pyridine-6-carbonitrile;
3-((1S,3R)-3-((5-cyano-4-(1-(2,2,2-trifluoroethyl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-(tetrahydro-2H-pyran-4-yl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-methyl-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-phenyl-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-(oxetan-3-yl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-(2-hydroxypropyl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-(2-hydroxyethyl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-(cyclopropylmethyl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-(2-fluoroethyl)-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-cyclopropyl-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-isopropyl-1H-pyrazol-4-yl)pyrimidin-2-yl)amino)cyclohexyl)-3H-imidazo[4,5-b]pyridine-6-carbonitrile;
3-((1S,3R)-3-((5-cyano-4-(1-ethyl-1H-pyrazol-4-yl)pyrimidin-2-yl)amino)cyclohexyl)-3H-imidazo[4,5-b]pyridine-6-carbonitrile;
2-(((1R,3S)-3-(6-(4-acetylpiperazin-1-yl)-3H-imidazo[4,5-b]pyridin-3-yl)cyclohexyl)amino)-4-(1-(2,2-difluoroethyl)-1H-pyrazol-4-yl)pyrimidine-5-carbonitrile;
4-(1-(2,2-difluoroethyl)-1H-pyrazol-4-yl)-2-(((1R,3S)-3-(6-(4-methylpiperazin-1-yl)-3H-imidazo[4,5-b]pyridin-3-yl)cyclohexyl)amino)pyrimidine-5-carbonitrile;
4-(1-(2,2-difluoroethyl)-1H-pyrazol-4-yl)-2-(((1R,3S)-3-(6-morpholino-3H-imidazo[4,5-b]pyridin-3-yl)cyclohexyl)amino)pyrimidine-5-carbonitrile;
4-(1-(2,2-difluoroethyl)-1H-pyrazol-4-yl)-2-(((1R,3S)-3-(6-(4-methylpyridin-3-yl)-3H-imidazo[4,5-b]pyridin-3-yl)cyclohexyl)amino)pyrimidine-5-carbonitrile;
4-(1-(2,2-difluoroethyl)-1H-pyrazol-4-yl)-N-((1R,3S)-3-(6-(prop-1-en-2-yl)-3H-imidazo[4,5-b]pyridin-3-yl)cyclohexyl)-5-(trifluoromethyl)pyrimidin-2-amine;
N-((1R,3S)-3-(6-cyclopropyl-3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine;
N-((1R,3S)-3-(6-(1H-pyrazol-4-yl)-3H-imidazo[4,5-b]pyridin-3-yl)cyclohexyl)-4-(1-(2,2-difluoroethyl)-1H-pyrazol-4-yl)-5-(trifluoromethyl)pyrimidin-2-amine;
4-(1-(2,2-difluoroethyl)-1H-pyrazol-4-yl)-N-((1R,3S)-3-(6-(1-methyl-1H-pyrazol-4-yl)-3H-imidazo[4,5-b]pyridin-3-yl)cyclohexyl)-5-(trifluoromethyl)pyrimidin-2-amine;
4-(1-(2,2-difluoroethyl)-1H-pyrazol-4-yl)-N-((1R,3S)-3-(6-(pyridin-3-yl)-3H-imidazo[4,5-b]pyridin-3-yl)cyclohexyl)-5-(trifluoromethyl)pyrimidin-2-amine;
4-(1-(2,2-difluoroethyl)-1H-pyrazol-4-yl)-N-((1R,3S)-3-(6-(pyrrolidin-1-yl)-3H-imidazo[4,5-b]pyridin-3-yl)cyclohexyl)-5-(trifluoromethyl)pyrimidin-2-amine;
3-((1S,3R)-3-((4-(1-(2,2-difluoroethyl)-1H-pyrazol-4-yl)-5-(trifluoromethyl)pyrimidin-2-yl)amino)cyclohexyl)-N,N-dimethyl-3H-imidazo[4,5-b]pyridin-6-amine;
4-(1-(2,2-difluoroethyl)-1H-pyrazol-4-yl)-2-(((1R,3S)-3-(4-methyl-1H-imidazo[4,5-c]pyridin-1-yl)cyclohexyl)amino)pyrimidine-5-carbonitrile;
1-((1S,3R)-3-((5-cyano-4-(1-(2,2-difluoroethyl)-1H-pyrazol-4-yl)pyrimidin-2-yl)amino)cyclohexyl)-1H-benzo[d]imidazole-5-carbonitrile;
1-((1S,3R)-3-((4-(1-(2,2-difluoroethyl)-1H-pyrazol-4-yl)-5-(trifluoromethyl)pyrimidin-2-yl)amino)cyclohexyl)-1H-benzo[d]imidazole-7-carbonitrile;
3-((1S,3R)-3-((5-cyano-4-(1-(2,2-difluoroethyl)-1H-pyrazol-4-yl)pyrimidin-2-yl)amino)cyclohexyl)-3H-imidazo[4,5-c]pyridine-6-carbonitrile; and
3-((1S,3R)-3-((5-cyano-4-(1,5-dimethyl-1H-pyrazol-4-yl)pyrimidin-2-yl)amino)cyclohexyl)-3H-imidazo[4,5-b]pyridine-6-carbonitrile;
or a pharmaceutically acceptable salt thereof.

31. A pharmaceutical composition comprising the compound of claim 2, or a pharmaceutically acceptable salt thereof, and a pharmaceutically acceptable carrier.

32. A method of inhibiting CDK12, comprising contacting the CDK12 with the compound of claim 2, or a pharmaceutically acceptable salt thereof.

33. A method of inhibiting CDK12 in a patient, comprising administering to the patient the compound of claim 2, or a pharmaceutically acceptable salt thereof.

34. A method of treating a disease or disorder associated with CDK12 in a patient, comprising administering to the patient a therapeutically effective amount of the compound of claim 2, or pharmaceutically acceptable salt thereof.

35. The method of claim 34, wherein the disease or disorder is cancer.

36. The method of claim 35, wherein the disease or disorder is a cancer which has been previously identified as homologous recombination deficiency (HRD) high.

37. The method of claim 34, wherein the cancer is ovarian cancer, breast cancer, Ewing's sarcoma, osteosarcoma, liver cancer, hepatocellular carcinoma, or colorectal cancer.


SEQUENCE LISTING (2 sequences)

SEQ ID NO: 1 - protein, 19 aa, synthetic construct
DYSTTPGGTLFSTTPGTRI

SEQ ID NO: 2 - protein, 8 aa, synthetic construct
VTPRTPPP